(19) United States
(12) Patent Application Publication
(10) Pub. No.: US 2026/0103468 A1
(43) Pub. Date: Apr. 16, 2026

(54) FUSED RING COMPOUND, PHARMACEUTICAL COMPOSITION, AND USE THEREOF

(71) Applicant: Hainan Simcere Zaiming Pharmaceutical Co., Ltd., Haikou, Hainan (CN)

(72) Inventors: Jinghai Jin, Shanghai (CN); Yuewen Li, Shanghai (CN); Yu Shi, Shanghai (CN); Wei Zhu, Shanghai (CN); Zhengtao Li, Shanghai (CN)

(73) Assignee: Hainan Simcere Zaiming Pharmaceutical Co., Ltd., Haikou, Hainan (CN)

(21) Appl. No.: 19/116,175

(22) PCT Filed: Sep. 28, 2023
(86) PCT No.: PCT/CN2023/122509

§ 371 Date: Mar. 27, 2025

(30) Foreign Application Priority Data
Sep. 29, 2022 (CN) 202211200084.4
Dec. 30, 2022 (CN) 202211725524.8
Jun. 21, 2023 (CN) 202310747025.7

(51) Int. Cl. C07D 487/04; A61K 31/454; A61P 35/00

(52) U.S. Cl. CPC C07D 487/04; A61K 31/454; A61P 35/00

(57) ABSTRACT

The present disclosure provides a compound represented by formula (I) or a pharmaceutically acceptable salt thereof, a pharmaceutical composition containing the compound and the salt thereof, and uses thereof, which are particularly suitable for preparing a medicament for treating or preventing abnormal cell proliferation diseases.

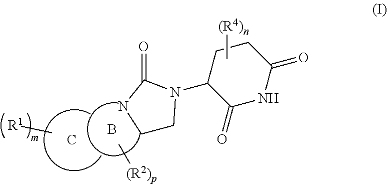

### CROSS-REFERENCE TO RELATED APPLICATION

The present disclosure claims the priority of the following applications: patent application No. 202211200084.4 entitled “A class of fused ring compounds, pharmaceutical compositions and uses thereof” and filed with the China National Intellectual Property Administration on Sep. 29, 2022; patent application No. 202211725524.8 entitled “A class of fused ring compounds, pharmaceutical compositions and their uses thereof” and filed with the China National Intellectual Property Administration on Dec. 30, 2022; and patent application No. 202310747025.7 entitled “A class of fused ring compounds, pharmaceutical compositions and their uses thereof” and filed with China National Intellectual Property Administration on Jun. 21, 2023. The foregoing prior applications are incorporated into the present disclosure in their entireties by reference.

### TECHNICAL FIELD

The present disclosure belongs to the technical field of medicine, and particularly relates to small molecule compound or a pharmaceutically acceptable salt thereof capable of regulating CRBN and degrading the specific protein, wherein the compound or the pharmaceutically acceptable salt thereof has biological activities such as anti-proliferation of tumor cells, and is used for the treatment of related diseases.

### BACKGROUND

Ubiquitin-proteasome system (UPS) is one of the important protein degradation pathways in human cells, which mainly includes two processes: ubiquitination of substrate protein and degradation of ubiquitin-labeled protein by proteasome. The abnormal function of ubiquitin-proteasome system will affect the cell cycle regulation, cell growth, proliferation, apoptosis, DNA repair and other cell signal transduction processes, and is closely related to the occurrence and development of malignant tumors, cardiovascular diseases, neurodegenerative diseases, etc. The use of small molecules in the targeted degradation of specific proteins through the ubiquitin-proteasome system has become one of the hot fields in the treatment of related diseases. One of them is the ability to bind to the E3 ligase to change the protein conformation of the E3 ligase, thereby inducing or stabilizing the protein-protein interaction between the E3 ligase and its substrate specific protein, and inducing the degradation of the specific protein. For example, the immunomodulators thalidomide, lenalidomide, and pomalidomide can induce the degradation of proteins such as IKZF1/3, CK1α, and GSPT1 after binding to CRBN. Although these marketed drugs mainly show good efficacy in hematological tumors and are widely used, there is still a need for further development of small molecule compounds that can both regulate CRBN and degrade specific proteins for the treatment of other types of tumors.

### SUMMARY OF THE INVENTION

The present disclosure provides a compound of formula (I) or a pharmaceutically acceptable salt thereof,
wherein,

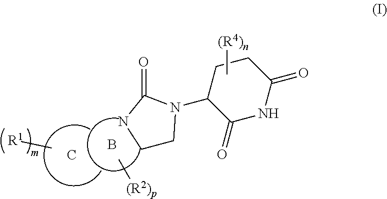

ring B is selected from 5- to 6-membered heteroaromatic ring and 5- to 8-membered heterocyclic ring;
ring C is selected from 5- to 6-membered heteroaromatic ring, 5- to 8-membered heterocyclic ring, benzene ring, and C₅-C₈ saturated or partially saturated carbocyclic ring;
R¹ and R² are each independently selected from the following group;
(a) halogen, ═O, CN, NO₂, —OR^{b}, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, or 5- to 10-membered heteroaryl is optionally substituted with R^{a};
or,
(b)

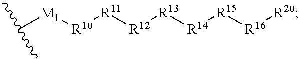

M₁ is selected from bond, —NR^{b}—, —C(O)—, —C(O)O—, —SO₂—, —S(O)—, —O—, —S—, —C(O)NR^{b}—, —C(═NR^{b})—, 2- to 10-membered heteroalkylene, C₁-C₁₀ alkylene, C₂-C₁₀ alkynylene, C₂-C₁₀ alkenylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkynylene, C₂-C₁₀ alkenylene, C₃-C₁₀ cycloalkylene, 4- to 9-membered heterocyclylene, C₆-C₁₀ arylene, or 5- to 10-membered heteroarylene is optionally substituted with R^{a};
R¹⁰, R¹¹, R¹², R¹³, R¹⁴, R¹⁵, and R¹⁶ are independently selected from bond, —NR^{b}—, —C(O)—, —C(O)O—, —SO₂—, —S(O)—, —O—, —S—, —NR^{b}C(O)—, —C(═NR^{b})—, —C(S)—, —P(O)(OR^{b})O—, —P(O)(OR^{b})—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R^{a};
R²⁰ is selected from H, halogen, CN, —OR^{b}, —N(R^{b})₂, —S(O)R^{b}, —SO₂R^{b}, —C(O)R^{b}, —C(O)OR^{b}, —OC(O)R^{b}, —C(O)N(R^{b})₂, —NR^{b}C(O)R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, or 5- to 10-membered heteroaryl is optionally substituted with R^{a};
each R⁴ is selected from halogen, CN, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{a};
each R^{a} is independently selected from halogen, CN, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{c};
each R^{b} is independently selected from H, halogen, CN, OH, NH₂, 2- to 10-membered heteroalkyl, C₁-C₁₀ alkyl, C₃-C₁₀ cycloalkyl, 4- to 8-membered heterocyclyl, C₁-C₁₀ aryl, and 5- to 10-membered heteroaryl, wherein the OH, NH₂, 2-10 membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{c};
each R^{c} is independently selected from halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{d};
each R^{d} is independently selected from halogen, CN, OH, NH₂, and C₁-C₆ alkyl;
n is independently selected from 0, 1, 2, 3, and 4;
m and p are independently selected from 0, 1, 2, 3, 4, 5, and 6.

In some embodiments, ring B is selected from 5- to 6-membered heteroaromatic ring and 5- to 6-membered heterocyclic ring.

In some embodiments, ring B is selected from 5- to 6-membered heteroaromatic ring.

In some embodiments, ring C is selected from 5- to 6-membered heteroaromatic ring, 5- to 6-membered heterocyclic ring, benzene ring, and C₅-C₆ saturated or partially saturated carbocyclic ring.

In some embodiments, ring C is selected from 5- to 6-membered heteroaromatic ring and benzene ring.

In some embodiments, ring C is selected from benzene ring, pyridine ring, pyrimidine ring, pyrazine ring, and thiophene ring.

In some embodiments, ring C is benzene ring.

In some embodiments, R¹ and R² are independently selected from halogen, CN, NO₂, —OR^{b}, —N(R^{b})₂, —S(O)R^{b}, —SO₂R^{b}, C₁-C₁₀ alkyl, and C₃-C₁₀ cycloalkyl, wherein the C₁-C₁₀ alkyl or C₃-C₁₀ cycloalkyl is optionally substituted with R^{a}.

In some embodiments, R¹ and R² are independently selected from halogen, CN, OH, NH₂, C₁-C₁₀ alkyl, and C₃-C₁₀ cycloalkyl, wherein the C₁-C₁₀ alkyl or C₃-C₁₀ cycloalkyl is optionally substituted with R.

In some embodiments, R¹⁰, R¹¹, R¹², R¹³, R¹⁴, R¹⁵, and R¹⁶ are independently selected from bond, —C(O)—, —C(O)O—, —O—, —S—, —C(O)NR^{b}—, —NR^{b}—, 2- to 10-membered heteroalkylene, C₁-C₁₀ alkylene, C₂-C₁₀ alkenylene, C₂-C₁₀ alkynylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, 4- to 9-membered heterocyclylene, C₆-C₁₀ arylene, or 5- to 10-membered heteroarylene is optionally substituted with R^{a}.

In some embodiments, R¹⁰ and R¹² are independently selected from bond, —C(O)O—, —O—, —S—, —C(O)NR^{b}—, —NR^{b}, C₁-C₁₀ alkylene, C₂-C₁₀ alkenylene, and C₂-C₁₀ alkynylene, wherein the C₁-C₁₀ alkylene, C₂-C₁₀ alkenylene, or C₂-C₁₀ alkynylene is optionally substituted with R^{a}.

In some embodiments, R¹⁰ and R¹² are independently selected from bond, —C(O)O—, —O—, —S—, —C(O)NR^{b}—, —NR^{b}, C₁-C₃ alkylene, and C₂-C₃ alkynylene, wherein the C₁-C₃ alkylene or C₂-C₃ alkynylene is optionally substituted with R^{a}.

In some embodiments, R¹⁰ and R¹² are independently selected from —O—, —NH—, CH₂, and C≡C, wherein the CH₂ is optionally substituted with R^{a}.

In some embodiments, R¹⁰ and R¹² are independently selected from —O—, —NH—, —CF₂—, —CH₂—, and —C≡C—.

In some embodiments, R¹³ is selected from C₃-C₁₀ cycloalkylene, 4- to 9-membered heterocyclylene, C₆-C₁₀ arylene, and 5- to 10-membered heteroarylene, wherein the C₃-C₁₀ cycloalkylene, 4- to 9-membered heterocyclylene, C₆-C₁₀ arylene, or 5- to 10-membered heteroarylene is optionally substituted with R^{a}.

In some embodiments, R¹³ is selected from C₃-C₆ cycloalkylene, 5- to 9-membered heterocyclylene, phenyl, and 5- to 6-membered heteroarylene, wherein the C₃-C₆ cycloalkylene, 5- to 9-membered heterocyclylene, phenyl, or 5- to 6-membered heteroarylene is optionally substituted with R^{a}.

In some embodiments, R¹³ is selected from phenyl and 5- to 6-membered heteroarylene, wherein the phenyl or 5- to 6-membered heteroarylene is optionally substituted with R^{a}.

In some embodiments, R¹³ is phenyl, wherein the phenyl is optionally substituted with R^{a}.

In some embodiments, R¹¹, R¹² and R¹³ are independently selected from bond, —C(O)—, —C(O)O—, —O—, —S—, —C(O)NR^{b}—, and —NR^{b}—.

In some embodiments, R¹⁴ is selected from bond, —O—, —NR^{b}—, —C(O)NR^{b}—, 2- to 6-membered heteroalkylene, C₁-C₆ alkylene, C₂-C₆ alkenylene, C₂-C₆ alkynylene, 4- to 6-membered heterocyclylene, C₆-C₁₀ arylene, and 5- to 6-membered heteroarylene, wherein the 2- to 6-membered heteroalkylene, C₁-C₆ alkylene, C₂-C₆ alkenylene, C₂-C₆ alkynylene, 4- to 6-membered heterocyclylene, C₆-C₁₀ arylene, or 5- to 6-membered heteroarylene is optionally substituted with R^{a}.

In some embodiments, R¹⁴ is selected from bond, —O—, —NR^{b}—, —C(O)NR^{b}—, 2- to 6-membered heteroalkylene, C₁-C₆ alkylene, C₂-C₆ alkenylene, and C₂-C₆ alkynylene, wherein the C₁-C₆ alkylene, C₂-C₆ alkenylene, or C₂-C₆ alkynylene is optionally substituted with R^{a}.

In some embodiments, R⁴ is selected from bond, —O—, —NR^{b}—, —C(O)NR^{b}—, 2- to 6-membered heteroalkylene, C₁-C₆ alkylene, C₂-C₆ alkenylene, and C₂-C₆ alkynylene.

In some embodiments, R¹ and R² are independently selected from

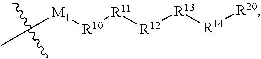

wherein M₁, R¹⁰, R¹¹, R¹², R¹³, R¹⁴, and R²⁰ are as defined above.

In some embodiments, R¹ and R² are independently selected from

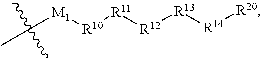

wherein R¹¹, R¹², and R¹³ are independently selected from bond, —C(O)—, —C(O)O—, —O—, —S—, —C(O)NR^{b}—, and —NR^{b}—; M₁, R¹⁰, R¹⁴, R²⁰, and R^{b} are as defined above.

In some embodiments, R¹ and R² are independently selected from

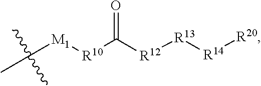

wherein M₁, R¹⁰, R¹², R¹³, R¹⁴, and R²⁰ are as defined above.

In some embodiments, R¹ and R² are independently selected from

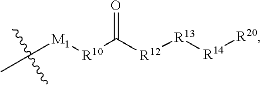

wherein R¹⁰ and R¹² are independently selected from bond, —C(O)—, —C(O)O—, —O—, —S—, —C(O)NR^{b}—, and —NR^{b}—; M₁, R¹³, R¹⁴, R²⁰, and R^{b} are as defined above.

In some embodiments R¹ and R² are independently selected from

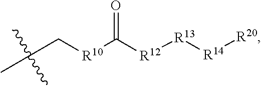

wherein R¹⁰ and R¹² are independently selected from bond, —C(O)O—, —O—, —S—, —C(O)NR^{b}—, —NR^{b}—, C₁-C₃ alkylene, and C₂-C₃ alkynylene, wherein the C₁-C₃ alkylene or C₂-C₃ alkynylene is optionally substituted with R^{a}; R¹³, R¹⁴, R²⁰, R^{b}, and R^{a} are as defined above.

In some embodiments, R¹ and R² are independently selected from

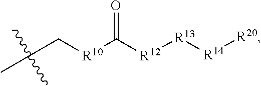

wherein R¹⁰ and R¹² are independently selected from —O—, —NH—, —CH₂—, and —C≡C—, wherein the —CH₂— is optionally substituted with R^{a}; R¹³, R¹⁴, R²⁰, and R^{a} are as defined above.

In some embodiments, R¹ and R² are independently selected from

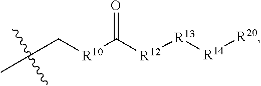

wherein R¹⁰ and R¹² are independently selected from —O—, —NH—, —CH₂—, and —C≡C—, wherein the —CH₂— is optionally substituted with R^{a}; R¹³ is selected from C₃-C₁₀ cycloalkylene, 4- to 9-membered heterocyclylene, C₆-C₁₀ arylene, and 5- to 10-membered heteroarylene, wherein the C₃-C₁₀ cycloalkylene, 4- to 9-membered heterocyclylene, C₆-C₁₀ arylene, or 5- to 10-membered heteroarylene is optionally substituted with R^{a}; R¹⁴, R²⁰, and R^{a} are as defined above.

In some embodiments, R¹ and R² are independently selected from

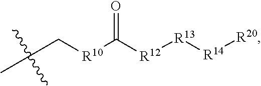

wherein R¹⁰ and R¹² are independently selected from —O—, —NH—, —CH₂—, —CF₂—, and —C≡C—; R¹³ is selected from C₃-C₆ cycloalkylene, 5- to 9-membered heterocyclylene, phenyl, and 5- to 6-membered heteroarylene, wherein the C₃-C₆ cycloalkylene, 5- to 9-membered heterocyclylene, phenyl, or 5- to 6-membered heteroarylene is optionally substituted with R^{a}; R¹⁴, R²⁰, and R^{a} are as defined above.

In some embodiments, R¹ and R² are independently selected from

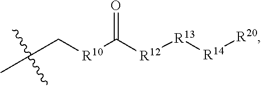

wherein R¹⁰ and R¹² are independently selected from bond, —C(O)O—, —O—, —S—, —C(O)NR^{b}—, —NR^{b}, C₁-C₃ alkylene, and C₂-C₃ alkynylene, wherein the C₁-C₃ alkylene or C₂-C₃ alkynylene is optionally substituted with R^{a}; R¹³ is selected from C₃-C₁₀ cycloalkylene, 4- to 9-membered heterocyclylene, C₆-C₁₀ arylene, and 5- to 10-membered heteroarylene, wherein the C₃-C₁₀ cycloalkylene, 4- to 9-membered heterocyclylene, C₆-C₁₀ arylene, or 5- to 10-membered heteroarylene is optionally substituted with R^{a}; R¹⁴, R²⁰, R^{b}, and R^{a} are as defined above.

In some embodiments, R¹ and R² are independently selected from

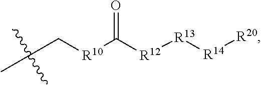

wherein R¹⁰ and R¹² are independently selected from —O—, —NH—, —CH₂—, and —C≡C—, wherein the —CH₂— is optionally substituted with R^{a}; R¹³ is selected from C₃-C₁₀ cycloalkylene, 4- to 9-membered heterocyclylene, C₆-C₁₀ arylene, and 5- to 10-membered heteroarylene, wherein the C₃-C₁₀ cycloalkylene, 4- to 9-membered heterocyclylene, C₆-C₁₀ arylene, or 5- to 10-membered heteroarylene is optionally substituted with R^{a}; R¹⁴ is selected from bond, —O—, —NR^{b}—, —C(O)NR^{b}—, 2- to 6-membered heteroalkylene, C₁-C₆ alkylene, C₂-C₆ alkenylene, and C₂-C₆ alkynylene, wherein the C₁-C₆ alkylene, C₂-C₆ alkenylene, or C₂-C₆ alkynylene is optionally substituted with R^{a}; R²⁰, R^{b}, and R^{a} are as defined above.

In some embodiments, R¹ and R² are independently selected from

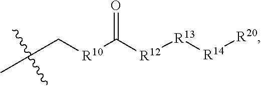

wherein R¹⁰ and R¹² are independently selected from —O—, —NH, CH₂, CF₂, and C≡C; R¹³ is selected from C₃-C₆ cycloalkylene, 5- to 9-membered heterocyclylene, phenyl, and 5- to 6-membered heteroarylene, wherein the C₃-C₆ cycloalkylene, 5-9 membered heterocyclylene, phenyl, or 5- to 6-membered heteroarylene is optionally substituted with R^{a}; R¹⁴ is selected from bond, —O—, —NR^{b}—, —C(O)NR^{b}—, 2- to 6-membered heteroalkylene, C₁-C₆ alkylene, C₂-C₆ alkenylene, and C₂-C₆ alkynylene, wherein the C₁-C₆ alkylene, C₂-C₆ alkenylene, or C₂-C₆ alkynylene is optionally substituted with R^{a}; R²⁰ is selected from H, —N(R^{b})₂, C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, and 5- to 10-membered heteroaryl, wherein the C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, or 5- to 10-membered heteroaryl is optionally substituted with R^{a}; R^{b} and R^{a} are as defined above.

In some embodiments, R¹ and R² are independently selected from the following group:

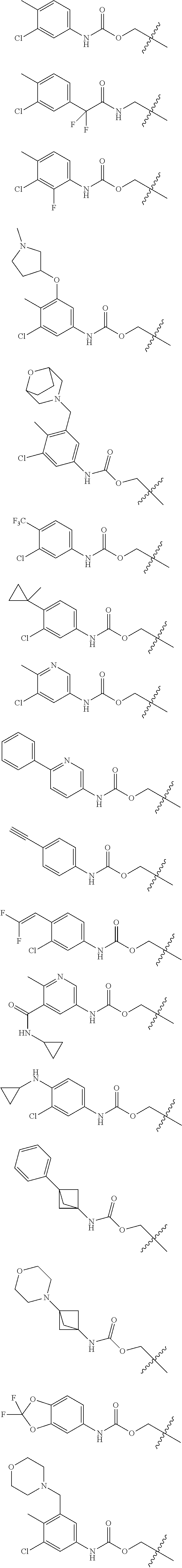

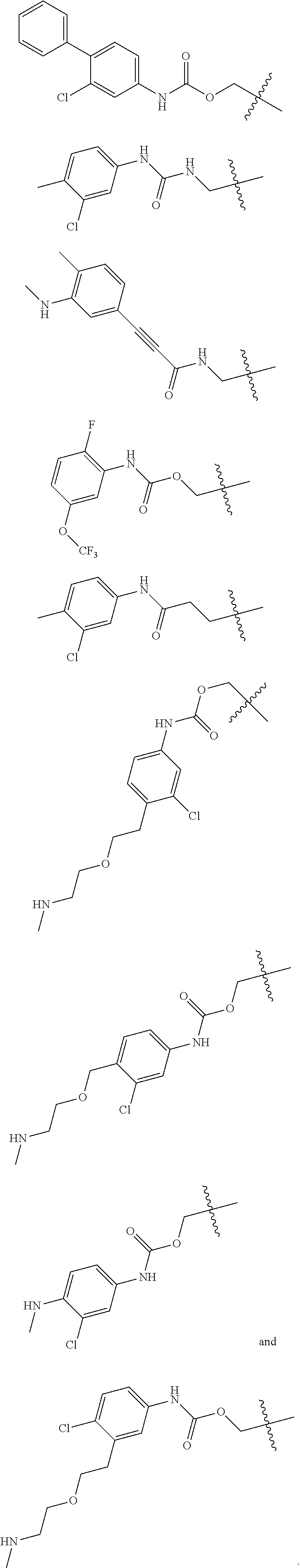

In some embodiments, R¹ and R² are independently selected from the following group:

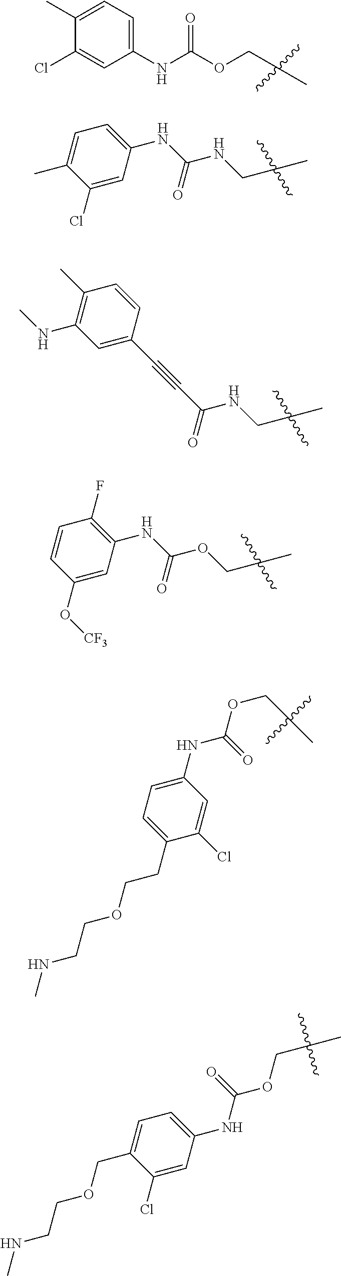 [SMILES: *COC(=O)Nc1ccc(C)c(Cl)c1 ; *CNC(=O)Nc1ccc(C)c(Cl)c1 ; *CNC(=O)C#Cc1ccc(C)c(NC)c1 ; *COC(=O)Nc1cc(OC(F)(F)F)ccc1F ; *COC(=O)Nc1ccc(CCOCCNC)c(Cl)c1 ; *COC(=O)Nc1ccc(COCCNC)c(Cl)c1]

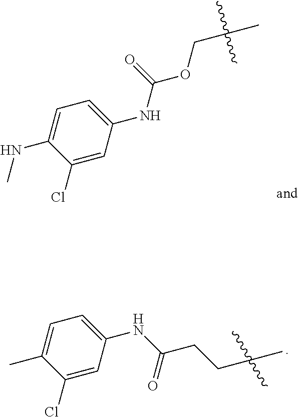 [SMILES: *COC(=O)Nc1ccc(NC)c(Cl)c1 ; *CCC(=O)Nc1ccc(C)c(Cl)c1]

In some embodiments, R¹ and R² are independently selected from

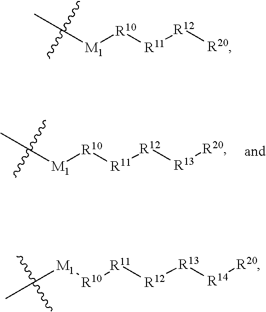

wherein M₁, R¹⁰, R¹¹, R¹², R¹³, R¹⁴, and R²⁰ are as defined above.

In some embodiments, M₁ is selected from bond, —NH—, —CH₂—, —CH₂CH₂—, —C(O)—, —C(O)O—, —O—, —S—, and —C(O)NH—.

In some embodiments, M₁ is selected from bond, —CH₂—, and —CH₂CH₂—.

In some embodiments, M₁ is —CH₂—.

In some embodiments, R²⁰ is selected from H, —N(R^{b})₂, 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, or 5- to 10-membered heteroaryl is optionally substituted with R^{a}.

In some embodiments, R²⁰ is selected from H, 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, or 5- to 10-membered heteroaryl is optionally substituted with R^{a}.

In some embodiments, R²⁰ is selected from H, —N(R^{b})₂, C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, and 5- to 10-membered heteroaryl, wherein the C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, or 5- to 10-membered heteroaryl is optionally substituted with R^{a}.

In some embodiments, R²⁰ is selected from H, —N(R^{b})₂, C₁-C₆ alkyl, C₂-C₆ alkenyl, C₂-C₆ alkynyl, C₃-C₆ cycloalkyl, 4- to 9-membered heterocyclyl, C₆-C₁₀ aryl, and 5- to 6-membered heteroaryl, wherein the C₁-C₆ alkyl, C₂-C₆ alkenyl, C₂-C₆ alkynyl, C₃-C₆ cycloalkyl, 4- to 9-membered heterocyclyl, C₆-C₁₀ aryl, or 5- to 6-membered heteroaryl is optionally substituted with R^{a}.

In some embodiments, R²⁰ is selected from H, C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, and 5- to 10-membered heteroaryl, wherein the C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, or 5- to 10-membered heteroaryl is optionally substituted with R^{a}.

In some embodiments, R⁴ is independently selected from halogen, CN, OH, NH₂, 2- to 10-membered heteroalkyl, and C₁-C₁₀ alkyl, wherein the OH, NH₂, 2- to 10-membered heteroalkyl, or C₁-C₁₀ alkyl is optionally substituted with R^{a}.

In some embodiments, each R^{a} is independently selected from halogen. CN, OH, NH₂, C₁-C₁₀ alkyl, C₃-C₁₀ cycloalkyl, and 4- to 8-membered heterocyclyl, wherein the OH, NH₂, C₁-C₁₀ alkyl, C₃-C₁₀ cycloalkyl, or 4- to 8-membered heterocyclyl is optionally substituted with R^{c}.

In some embodiments, each R^{a} is independently selected from halogen, CN, OH, NH₂, and C₁-C₁₀ alkyl, wherein the OH, NH₂, or C₁-C₁₀ alkyl is optionally substituted with R^{c}.

In some embodiments, each R^{a} is independently selected from halogen and C₁-C₆ alkyl, wherein the C₁-C₆ alkyl is optionally substituted with R^{c}.

In some embodiments, each R^{a} is independently selected from F, Cl, CH₃, and CF₃.

In some embodiments, each R^{b} is independently selected from H, C₁-C₆ alkyl, C₃-C₁₀ cycloalkyl, 4- to 8-membered heterocyclyl, C₆-C₁₀ aryl, and 5- to 10-membered heteroaryl, wherein the C₁-C₆ alkyl, C₃-C₁₀ cycloalkyl, 4- to 8-membered heterocyclyl, C₆-C₁₀ aryl, or 5- to 10-membered heteroaryl is optionally substituted with R^{c}.

In some embodiments, each R^{b} is independently selected from H and C₁-C₆ alkyl, wherein the C₁-C₆ alkyl is optionally substituted with R^{c}.

In some embodiments, each R^{b} is independently H.

In some embodiments, each R^{c} is independently selected from halogen, CN, OH, NH₂, and C₁-C₆ alkyl, wherein the C₁-C₆ alkyl is optionally substituted with R^{d}.

In some embodiments, each R^{c} is independently selected from halogen, CN, OH, NH₂, and C₁-C₆ alkyl.

In some embodiments, each R^{d} is independently halogen.

In some embodiments, m and p are independently selected from 0, 1, and 2.

In some embodiments, m and p are independently selected from 0 and 1.

In some embodiments, m is 0, and p is 1.

In some embodiments, m is 1, and p is 0.

In some embodiments, n is selected from 0 and 1.

In some embodiments, n is 0.

In some embodiments, the compound of formula (I) or the pharmaceutically acceptable salt thereof of the present disclosure is selected from a compound of formula (II) or a pharmaceutically acceptable salt thereof,

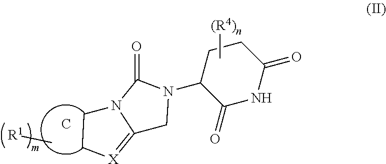

wherein, X is selected from N and CH, wherein the CH is optionally substituted with R²; ring C, R¹, R², R⁴, m, and n are as defined above.

In some embodiments, the compound of formula (II) or the pharmaceutically acceptable salt thereof of the present disclosure is selected from a compound of formula (II-1) or (II-2) or a pharmaceutically acceptable salt thereof,

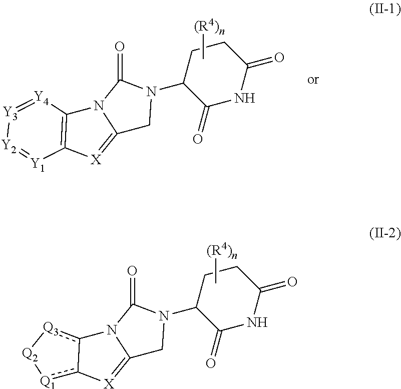

wherein, “ [symbol] ” represents a single bond or a double bond; X is selected from N and CH, wherein the CH is optionally substituted with R²; Y₁, Y₂, Y₃, and Y₄ are independently selected from N and CH, wherein the CH is optionally substituted with R¹; Q₁, Q₂, and Q₃ are independently selected from O, S, NH, CH₂, N, and CH, wherein the NH, CH₂, or CH is optionally substituted with R¹; R¹, R², R⁴, and n are as defined above.

In some embodiments, “ [symbol] ” is a double bond, Q₂ is selected from O, S, NH, and CH₂; and Q₁ and Q₃ are independently selected from N and CH, wherein the NH, CH₂, or CH is optionally substituted with R¹.

In some embodiments, the compound of formula (II) or the pharmaceutically acceptable salt thereof of the present disclosure is selected from a compound of formula (II-1a) or a pharmaceutically acceptable salt thereof,

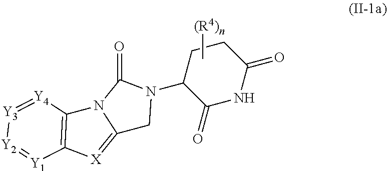

wherein, X is selected from N and CH, wherein the CH is optionally substituted with R²; Y₁, Y₃, and Y₄ are independently selected from N and CH, wherein the CH is optionally substituted with R¹; Y₂ is CH, wherein the CH is optionally substituted with R¹; R¹, R², R⁴, and n are as defined above.

In some embodiments, Y₁, Y₂, Y₃, and Y₄ are independently CH, wherein the CH is optionally substituted with R¹.

In some embodiments, X is CH, wherein the CH is optionally substituted with R².

Without conflict, it should be understood that the above-described embodiments can be arbitrarily combined to form a technical solution that includes the features of the combined embodiments. Such combined technical solutions are within the scope of the present disclosure.

In some embodiments, the compound of formula (I) or the pharmaceutically acceptable salt thereof is selected from the following compounds or pharmaceutically acceptable salts thereof,

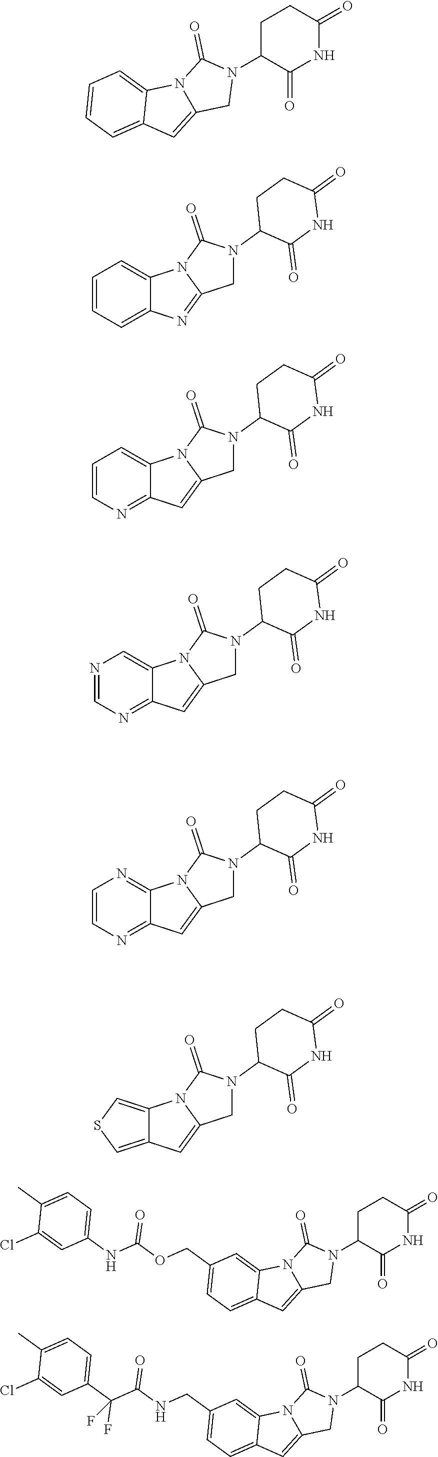

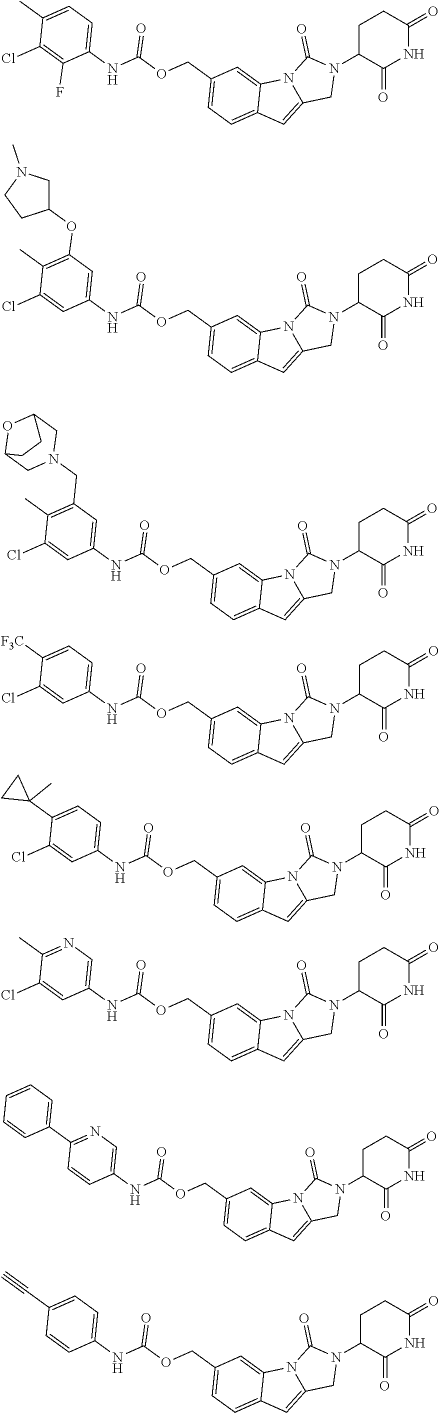

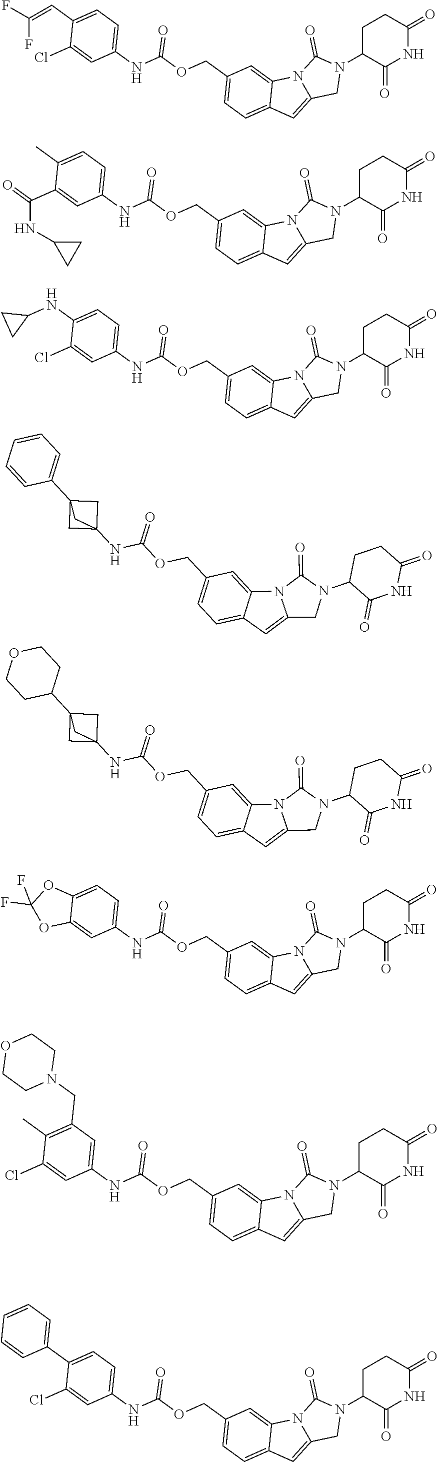

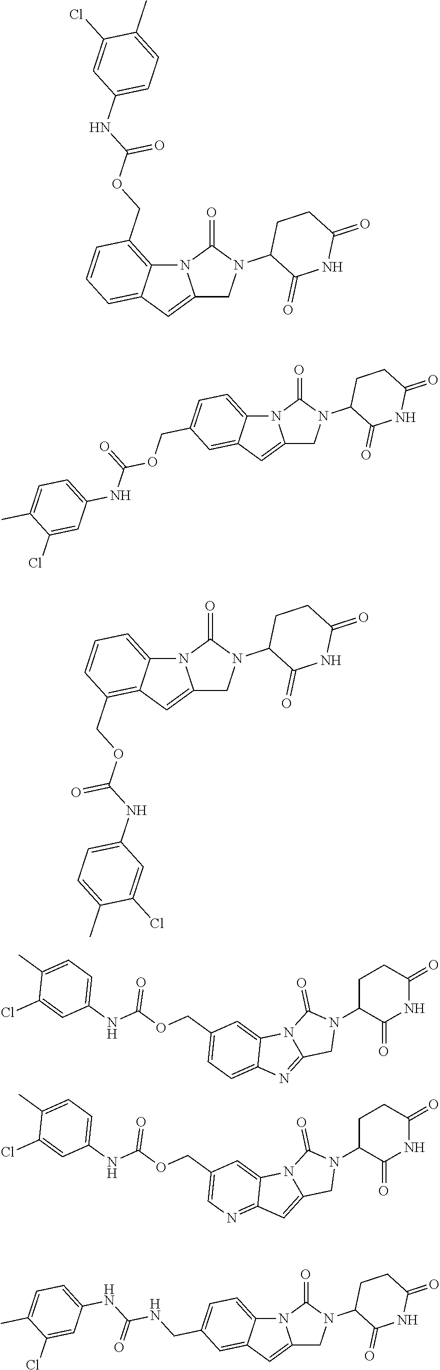

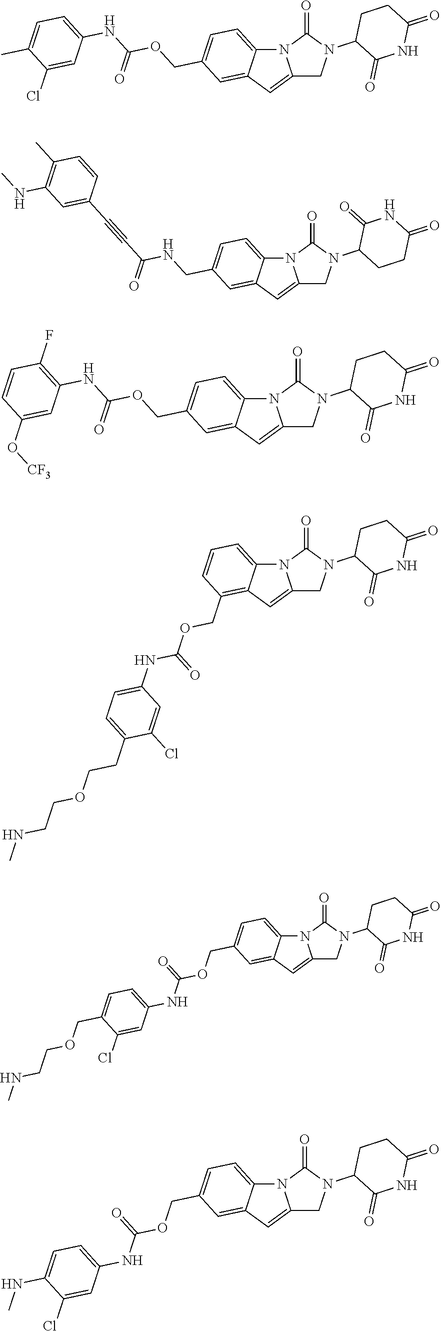 [SMILES: Cc1ccc(NC(=O)OCc2ccc3c(c2)cc2n3C(=O)N(C3CCC(=O)NC3=O)C2)cc1Cl ; CNc1cc(C#CC(=O)NCc2ccc3c(c2)cc2n3C(=O)N(C3CCC(=O)NC3=O)C2)ccc1C ; O=C1CCC(N2Cc3cc4cc(COC(=O)Nc5cc(OC(F)(F)F)ccc5F)ccc4n3C2=O)C(=O)N1 ; CNCCOCCc1ccc(NC(=O)OCc2cccc3c2cc2n3C(=O)N(C3CCC(=O)NC3=O)C2)cc1Cl ; CNCCOCc1ccc(NC(=O)OCc2ccc3c(c2)cc2n3C(=O)N(C3CCC(=O)NC3=O)C2)cc1Cl ; CNc1ccc(NC(=O)OCc2ccc3c(c2)cc2n3C(=O)N(C3CCC(=O)NC3=O)C2)cc1Cl]

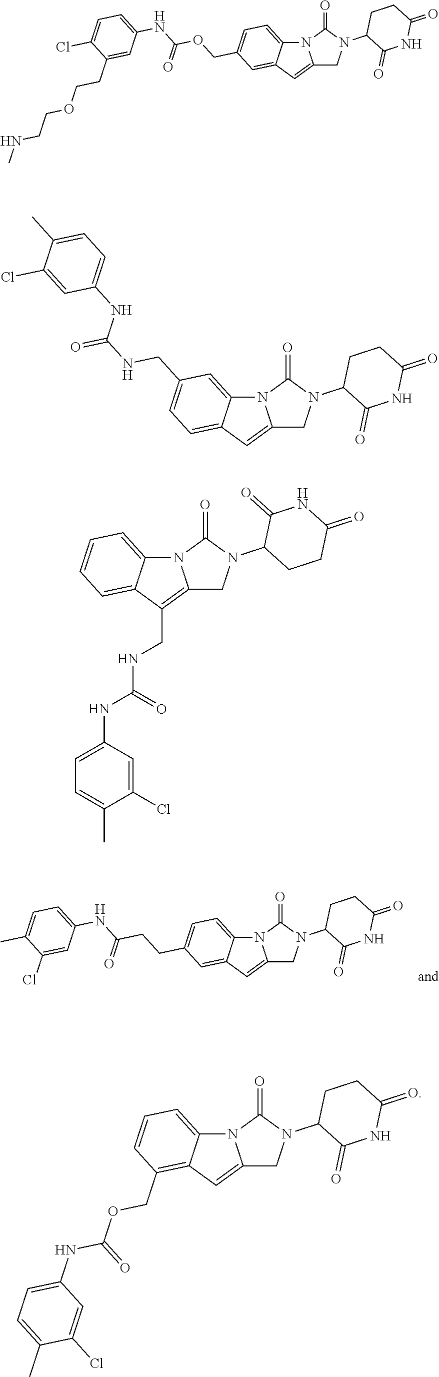 [SMILES: CNCCOCCc1cc(NC(=O)OCc2ccc3c(c2)cc2n3C(=O)N(C3CCC(=O)NC3=O)C2)ccc1Cl ; Cc1ccc(NC(=O)NCc2ccc3cc4n(c3c2)C(=O)N(C2CCC(=O)NC2=O)C4)cc1Cl ; Cc1ccc(NC(=O)NCc2c3n(c4ccccc24)C(=O)N(C2CCC(=O)NC2=O)C3)cc1Cl ; Cc1ccc(NC(=O)CCc2ccc3c(c2)cc2n3C(=O)N(C3CCC(=O)NC3=O)C2)cc1Cl ; Cc1ccc(NC(=O)OCc2cccc3c2cc2n3C(=O)N(C3CCC(=O)NC3=O)C2)cc1Cl]

In another aspect, the present disclosure provides a pharmaceutical composition, comprising the compound represented by formula (I) of the present disclosure, or the pharmaceutically acceptable salt thereof, and a pharmaceutically acceptable excipient.

In another aspect, the present disclosure provides a method for treating abnormal cell proliferation diseases in a mammal, comprising administering to a mammal, preferably a human, in need of the treatment a therapeutically effective amount of the compound represented by formula (I), or the pharmaceutically acceptable salt thereof, or the pharmaceutical composition comprising the same.

In another aspect, the present disclosure provides the use of the compound represented by formula (I) or the pharmaceutically acceptable salt thereof, or the pharmaceutical composition comprising the same in preparing a medicament for preventing or treating abnormal cell proliferation diseases.

In another aspect, the present disclosure provides the use of the compound represented by formula (I), or the pharmaceutically acceptable salt thereof, or the pharmaceutical composition comprising the same in preventing or treating abnormal cell proliferation diseases.

In another aspect, the present disclosure provides the compound represented by formula (I), or the pharmaceutically acceptable salt thereof, or the pharmaceutical composition comprising the same for use in preventing or treating abnormal cell proliferation diseases.

In some embodiments, the abnormal cell proliferation disease is cancer.

In some embodiments, the cancer is selected from solid tumors, adenocarcinoma, and hematoma.

### Terminology and Definitions

Unless otherwise stated, the terms used in the present disclosure have the following meanings, and definitions of groups and terms described in the present disclosure, including definitions thereof as examples, exemplary definitions, preferred definitions, definitions described in tables, definitions of specific compounds in the examples, and the like, can be arbitrarily combined and incorporated with each other. A specific term should not be regarded as uncertain or unclear unless specifically defined, but should be understood in accordance with its ordinary meaning in the art. When a trade name appears herein, it is intended to refer to its corresponding commercial product or its active ingredient thereof.

Herein,

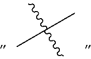

represents a linking site.

The graphical representation of racemates or enantiomerically pure compounds provided herein comes from Maehr, J. Chem. Ed. 1985, 62: 114-120. Unless otherwise specified, the wedge-shaped solid bond and the wedge-shaped dashed-line bond ( [symbol] and [symbol] ) are used to represent the absolute configuration of a stereogenic center, and the black solid-line bond and the dashed-line bond ( [symbol] and [symbol] ) are used to represent the absolute configuration of a stereogenic center (e.g. the cis- and trans-configuration of an alicyclic compound).

The term “tautomer” refers to functional isomers resulting from the rapid movement of a certain atom between two positions in the molecule. The compounds of the present disclosure may exhibit tautomerism. Tautomeric compounds may exist in two or more interconvertible forms. Tautomers generally exist in equilibrium. Trying to separate a single tautomer usually leads to a mixture, the physicochemical properties of the single tautomer are consistent with those of the mixture of compounds. The position of equilibrium depends on intramolecular chemical properties. For example, in many aliphatic aldehydes and ketones such as acetaldehyde, the keto-form predominates; whereas in phenol, the enol-form predominates. In the present disclosure, all tautomeric forms of compound are included.

The term “stereoisomer” refers to an isomer resulting from a difference spatial arrangement of atoms in a molecule, including cis-trans-isomers, enantiomers, and diastereomers.

The compounds of the present disclosure may have asymmetric atom(s) such as carbon atom(s), sulfur atom(s), nitrogen atom(s), phosphorus atom(s), or asymmetric double bond(s), and thus the compound of the present disclosure may exist in the form of a particular geometric isomer or stereoisomer. The form of a particular geometric isomer or stereoisomer may be cis- and trans-isomers. E and Z geometric isomers, (−)- and (+)-enantiomers, (R)- and (S)-enantiomers, diastereomers, (D)-isomers, (L)-isomers, and racemic or other mixtures thereof, such as enantiomerically or diastereomerically enriched mixtures, and all of the above isomers, as well as mixtures thereof, are encompassed within the definition scope of the compound of the present disclosure. Additional asymmetric carbon atom(s), asymmetric sulfur atom(s), asymmetric nitrogen atom(s) or asymmetric phosphorus atom(s) may be present in substituent(s) such as alkyl. All of these isomers and mixtures thereof referred to in all substituents are also encompassed within the definition scope of the compound of the present disclosure. The compound containing asymmetric atom(s) of the present disclosure can be separated in an enantiomerically pure form or a racemic form. The enantiomerically active pure form can be obtained by resolving racemic mixtures or by synthesis using chiral starting materials or chiral reagents.

The term “substituted” means that any one or more hydrogen atoms on a specific atom are replaced with substituent(s), as long as the valence state of the specific atom is normal and the compound resulting from the substitution is stable. When the substituent is oxo (i.e. ═O), it means that two hydrogen atoms are substituted and oxo is not available on an aromatic group.

The term “optionally” or “optionally” means that the subsequently described event or circumstance may or may not occur. The description includes instances where the event or circumstance occurs and instances where the event or circumstance does not occur. For example, ethyl being “optionally” substituted with halogen means that ethyl may be unsubstituted (CH₂CH₃), mono-substituted (CH₂CH₂F, CH₂CH₂Cl, etc.), polysubstituted (CHFCH₂F, CH₂CHF₂, CHFCH₂Cl, CH₂CHCl₂, etc.) or fully substituted (CF₂CF₃, CF₂CCl₃, CCl₂CCl₃, etc.). It will be understood by those skilled in the art that for any group comprising one or more substituents, no substitution or substituting pattern that is sterically impossible to exist and/or cannot be synthesized will be introduced.

When any variable (e.g. R^{a}, or R^{b}) appears more than once in the composition or structure of a compound, the variable is independently defined in each case. For example, if a group is substituted with two R^{b}, then the definition of each R^{b} is independent.

When one of the variables is a chemical bond, or is absent, it means that the two groups which it links are directly linked. For example, when L in A-L-Z represents a bond, it means that the structure is actually A-Z.

When the linking direction of the linking group referred to herein is not specified, the linking direction is arbitrary. For example, when L¹ in the structural unit

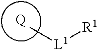

is “C₁-C₃ alkylene-O”, then L¹ may form “ring Q-C₁-C₃ alkylene-O—R¹” by connecting ring Q and R¹ in a direction from left to right, or may form “ring Q-O—C₁-C₃ alkylene-R¹” by connecting ring Q and R¹ in a direction from right to left.

When the bond of a substituent is cross-linked to two atoms on a ring, the substituent may be bonded with any atom on the ring. For example, the structural unit

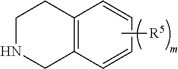

represents that R⁵ may be substituted at any position on the benzene ring.

Cₘ-Cₙ used herein means that it has an integer number of carbon atoms in the range of m-n. For example, “C₁-C₁₀” means that the group may have 1, 2, 3, 4, 5, 6, 7, 8, 9, or 10 carbon atoms.

Herein, the bond “ [symbol] ” depicted by solid and dashed lines represents a single bond or a double bond. For example, structural unit

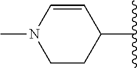

comprises

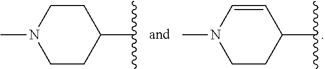

The term “alkyl” refers to a hydrocarbon group with a general formula of CₙH₂ₙ₊₁, which may be linear or branched. The term “C₁-C₁₀ alkyl” may be understood to represent a linear or branched saturated hydrocarbon group having 1, 2, 3, 4, 5, 6, 7, 8, 9 or 10 carbon atoms. Specific examples of such alkyl groups include, but are not limited to, methyl, ethyl, propyl, butyl, pentyl, hexyl, isopropyl, isobutyl, sec-butyl, tert-butyl, isopentyl, 2-methylbutyl, 1-methylbutyl, 1-ethylbutyl, 1,2-dimethylpropyl, neopentyl, 1,1-dimethylpropyl, 4-methylpentyl, 3-methylpentyl, 2-methylpentyl, 1-methylpentyl, 2-ethylbutyl, 1-ethylbutyl, 3,3-dimethylbutyl, 2,2-dimethylbutyl, 1,1-dimethylbutyl, 2,3-dimethylbutyl, 1,3-dimethylbutyl, 1,2-dimethylbutyl, and the like. The term “C₁-C₆ alkyl” can be understood to represent an alkyl group having 1, 2, 3, 4, 5 or 6 carbon atoms. Specific examples include, but are not limited to, methyl, ethyl, n-propyl, isopropyl, n-butyl, isobutyl, sec-butyl, tert-butyl, n-pentyl, 1-methylbutyl, 2-methylbutyl, 3-methylbutyl, neopentyl, hexyl, 2-methylpentyl, and the like. The term “C₁-C₃ alkyl” may be understood to represent a linear or branched saturated alkyl group having 1, 2 or 3 carbon atoms. The “C₁-C₁₀ alkyl” may include the ranges of “C₁-C₆ alkyl”, “C₁-C₃ alkyl” and the like, and the “C₁-C₆ alkyl” may further include “C₁-C₃ alkyl”.

The term “alkylene” refers to a residue derived from an “alkyl” group further removing one hydrogen atom.

The term “heteroalkyl” refers to an alkyl group comprising 1, 2, 3, 4, or 5 heteroatoms or heteroatom groups. The “heteroatom or heteroatom group” includes, but is not limited to, a nitrogen atom (N), an oxygen atom (O), a sulfur atom (S), a boron atom (B), a phosphorus atom (P), —S(═O)₂—, —S(═O)—, —NH—, and the like. The term “2- to 10-membered heteroalkyl” may be understood to represent a heteroalkyl group having 2, 3, 4, 5, 6, 7, 8, 9 or 10 atoms (carbon and heteroatoms other than hydrogen). The term “2- to 6-membered heteroalkyl” may be understood to represent heteroalkyl having 2, 3, 4, 5 or 6 atoms (carbon and heteroatoms other than hydrogen). The heteroalkyl group may be attached to other groups through heteroatoms or carbon atoms therein. The heteroatom may be located at any internal position of the heteroalkyl group (including the position where the heteroalkyl group is attached to another group). i.e. the heteroalkyl group does not include hydroxyalkyl (e.g. —CH₂OH, or —CH(CH₃)OH), aminoalkyl (e.g. —CH₂NH—, or —CH(CH₃)NH₂), and the like. Examples of heteroalkyl include, but are not limited to, —OCH₃, —OCH₂CH₃, —OCH₂(CH₃)₂, —CH₂CH₂OCH₃, —NHCH₃, —N(CH₃)₂, —NHCH₂CH₃, —CH₂—CH₂—NH—CH₃, —OCH₂—CH₂—N—HCH₃—, —OCH₂—CH₂—NH—CH(CH₃)₂, —SCH₃, —SCH₂CH₃—, —S(═O)—CH₃—, —CH₂—S(═O)₂CH₃, —CH₂C(═O)NH—CH₂—OCH₃, and the like.

The term “heteroalkylene” refers to a residue derived from a heteroalkyl group further removing one hydrogen atom.

The term “alkenyl” refers to a linear or branched, unsaturated aliphatic hydrocarbon group consisting of carbon atoms and hydrogen atoms and having at least one double bond. The term “C₂-C₁₀ alkenyl” can be understood to represent a linear or branched, unsaturated hydrocarbon group comprising one or more double bonds and having 2, 3, 4, 5, 6, 7, 8, 9 or 10 carbon atoms. The “C₂-C₁₀ alkenyl” is preferably “C₂-C₆ alkenyl”, further preferably “C₂-C₄ alkenyl”, and still further preferably C₂ or C₃ alkenyl. It will be understood that in the case that the alkenyl comprises more than one double bond, the double bonds may be isolated from one another or conjugated. Specific examples of the alkenyl include, but are not limited to, vinyl, allyl, (E)-2-methylvinyl, (Z)-2-methylvinyl, (E)-but-2-enyl, (Z)-but-2-enyl, (E)-but-1-enyl, (Z)-but-1-enyl, isopropenyl, 2-methylprop-2-enyl, 1-methylprop-2-enyl, 2-methylprop-1-enyl, (E)-1-methylprop-1-enyl, or (Z)-1-methylprop-1-enyl, and the like.

The term “alkenylene” refers to a residue derived from an alkenyl group further removing a hydrogen atom.

The term “alkynyl” refers to a linear or branched, unsaturated aliphatic hydrocarbon group consisting of carbon atoms and hydrogen atoms and having at least one triple bond. The term “C₂-C₁₀ alkynyl” may be understood to represent a linear or branched, unsaturated hydrocarbon group, comprising one or more triple bonds and having 2, 3, 4, 5, 6, 7, 8, 9 or 10 carbon atoms. Examples of “C₂-C₁₀ alkynyl” include, but are not limited to, ethynyl (—C≡CH), propynyl (—C≡CCH₃, —CH₂C≡CH), but-1-ynyl, but-2-ynyl, and but-3-ynyl. “C₂-C₁₀ alkynyl” may include “C₂-C₃ alkynyl”, and examples of “C₂-C₃ alkynyl” include ethynyl (—C≡CH), propyl-1-alkynyl (—C≡CCH₃), and propyl-2-alkynyl (—CH₂C≡CH).

The term “alkynylene” refers to a residue derived from an alkynyl group further removing a hydrogen atom.

The term “cycloalkyl” refers to a carbocyclic ring that is fully saturated and exists in the form of a monocyclic ring, fused ring, bridged ring, spirocyclic ring, and the like. Unless otherwise indicated, the carbocyclic ring is generally a 3- to 10-membered ring. The term “C₃-C₁₀ cycloalkyl” may be understood to represent a saturated monocyclic, fused, spiro, or bridged ring having 3, 4, 5, 6, 7, 8, 9 or 10 carbon atoms. Specific examples of the cycloalkyl include, but are not limited to, cyclopropyl, cyclobutyl, cyclopentyl, cyclohexyl, cycloheptyl, cyclooctyl, cyclononyl, cyclodecyl, norbornyl (bicyclo [2.2.1] heptyl), bicyclo [2.2.2] octyl, adamantyl, spiro [4.5] decyl, and the like. The term “C₃-C₁₀ cycloalkyl” may include “C₃-C₆ cycloalkyl”, and the term “C₃-C₆ cycloalkyl” may be understood to represent a saturated monocyclic or bicyclic hydrocarbon ring having 3, 4, 5, or 6 carbon atoms. Specific examples including, but not limited to, cyclopropyl, cyclobutyl, cyclopentyl, or cyclohexyl, and the like. The term “C₅-C₈cycloalkyl” may be understood to represent a saturated monocyclic or bicyclic hydrocarbon ring having 5, 6, 7 or 8 carbon atoms, and may also be denoted as a C₅-C₈ saturated carbocyclic ring.

The term “partially saturated carbocyclic ring” refers to a non-aromatic carbocyclic ring that is not completely saturated and exists in the form of a monocyclic ring, fused ring, bridged ring, or spirocyclic ring, and is typically a 5- to 8-membered ring unless otherwise indicated. The term “C₅-C₈ partially saturated carbocyclic ring” may be understood to represent a partially saturated monocyclic, fused, spiro, or bridged ring having 5, 6, 7 or 8 carbon atoms. Specific examples of the C₅-C₈ partially saturated carbocyclic ring include, but are not limited to, cyclopentenyl, cyclopentadienyl, cyclohexenyl, cyclohexadienyl, cycloheptenyl, or cycloheptadienyl, and the like.

The term “cycloalkylene” refers to a residue derived from a cycloalkyl group further removing one hydrogen atom.

The term “heterocyclyl” refers to a fully saturated or partially saturated (but not an aromatic heteroaryl on the whole) monocyclic ring, fused ring, spirocyclic ring or bridged ring group, and the ring atoms of the group containing 1, 2, 3, 4 or 5 heteroatoms or heteroatom groups (i.e. heteroatom-containing groups). The “heteroatom or heteroatoms group” includes, but is not limited to, a nitrogen atom (N), an oxygen atom (O), a sulfur atom (S), a phosphorus atom (P), a boron atom (B), —S(═O)₂—, —S(═O)—, —P(═O)₂—, —P(═O)—, —NH—, —S(═O)(═NH)—, —C(═O)NH—, or —NHC(═O)NH—, and the like. The term “3- to 10-membered heterocyclyl” refers to a heterocyclyl having 3, 4, 5, 6, 7, 8, 9 or 10 ring atoms, the ring atoms therein containing 1, 2, 3, 4 or 5 heteroatoms or heteroatom groups independently selected from those described above. “3- to 10-membered heterocyclyl” includes “4- to 7-membered heterocyclyl”, wherein specific examples of 4-membered heterocyclyl include, but are not limited to, azetidinyl, thietanyl, or oxetanyl; specific examples of 5-membered heterocyclyl include, but are not limited to, tetrahydrofuranyl, dioxolenyl, pyrrolidinyl, imidazolidinyl, pyrazolidinyl, pyrrolinyl, 4, 5-dihydrooxazolyl, or 2, 5-dihydro-1H-pyrrolyl; specific examples of 6-membered heterocyclyl include, but are not limited to, tetrahydropyranyl, piperidyl, morpholinyl, dithialkyl, thiomorpholinyl, piperazinyl, trithialkyl, tetrahydropyridinyl, or 4H-[1,3,4] thiadiazinyl; specific examples of 7-membered heterocyclyl include, but are not limited to, diazepanyl. The heterocyclyl may also be a bicyclyl group, wherein specific examples of 5,5-membered bicyclyl group include, but are not limited to, hexahydrocyclopenta[c]pyrrol-2(1H)-yl; specific examples of 5,6-membered bicyclic groups include, but are not limited to, hexahydropyrrolo[1,2-a]pyrazin-2(1H)-yl, 5,6,7,8-tetrahydro-[1,2,4]triazolo[4,3-a]pyrazinyl, or 5,6,7,8-tetrahydroimidazo[1,5-a]pyrazinyl. Optionally, the heterocyclyl may be a benzo-fused ring group of the above-mentioned 4- to 7-membered heterocyclyl, and specific examples include, but are not limited to, dihydroisoquinolinyl and the like. The “4- to 10-membered heterocyclyl” may include the ranges of “5- to 10-membered heterocyclyl”, “4- to 7-membered heterocyclyl”, “5- to 6-membered heterocyclyl”, “6- to 8-membered heterocyclyl”, “4- to 10-membered heterocyclyl”, “4- to 7-membered heterocyclyl”, “5- to 6-membered heterocyclyl”, “6- to 8-membered heterocyclyl”, and the like. “4- to 7-membered heterocyclyl” may further include the ranges of “4- to 6-membered heterocyclyl”, “5- to 6-membered heterocyclyl”, “4- to 7-membered heterocycloalkyl”, “4- to 6-membered heterocycloalkyl”, and “5- to 6-membered heterocycloalkyl”, and the like. Although some bicyclic heterocyclyl herein comprise, in part, a benzene ring or a heteroaromatic ring, the heterocyclyl is still non-aromatic on the whole.

The term “heterocyclylene” refers to a residue derived from a heterocyclyl group further removing a hydrogen atom.

The term “aryl” refers to an aromatic all-carbon monocyclic or fused polycyclic group with a conjugated n-electron system. Aryl may have 6 to 20 carbon atoms, 6 to 14 carbon atoms, or 6 to 12 carbon atoms. The term “C₆-C₂₀ aryl” may be understood as an aryl group having 6 to 20 carbon atoms. In particular rings having 6 carbon atoms (“C₆ aryl”), such as phenyl; or a ring having 9 carbon atoms (“C₉ aryl”), such as indanyl or indenyl; or a ring having 10 carbon atoms (“C₁₀ aryl”), such as tetrahydronaphthyl, dihydronaphthyl, or naphthyl; or a ring having 13 carbon atoms (“C₁₃ aryl”), such as fluorenyl; or is a ring having 14 carbon atoms (“C14 aryl”), such as anthracenyl. The term “C₆-C₁₀ aryl” may be understood as an aryl group having 6 to 10 carbon atoms. In particular rings having 6 carbon atoms (“C₆ aryl”), such as phenyl; or a ring having 9 carbon atoms (“C₉ aryl”), such as indanyl or indenyl; or a ring having 10 carbon atoms (“C₁₀ aryl”), such as tetrahydronaphthyl, dihydronaphthyl, or naphthyl. The term “C₆-C₂₀ aryl” may include “C₆-C₁₀ aryl”.

The term “arylene” refers to a residue derived from an aryl group by further removing one hydrogen atom.

The term “heteroaryl” refers to an aromatic monocyclic or fused polycyclic ring system, containing at least one ring atom selected from N, O, S, with the remaining ring atoms being C. The term “5- to 10-membered heteroaryl” may be understood to include an aromatic monocyclic or bicyclic ring system having 5, 6, 7, 8, 9 or 10 ring atoms, particularly 5 or 6 or 9 or 10 ring atoms, and which contains 1, 2, 3, 4 or 5, preferably 1, 2 or 3 heteroatoms independently selected from N, O and S. In particular, the heteroaryl is selected from thienyl, furanyl, pyrrolyl, oxazolyl, thiazolyl, imidazolyl, pyrazolyl, isoxazolyl, isothiazolyl, oxadiazolyl, triazolyl, thiadiazolyl, and the like, and the benzo derivatives thereof, such as benzofuranyl, benzothienyl, benzothiazolyl, benzoxazolyl, benzisoxazolyl, benzimidazolyl, benzotriazolyl, indazolyl, indolyl, isoindolyl, and the like; and pyridinyl, pyridazinyl, pyrimidinyl, pyrazinyl, triazinyl, and the like, and the benzo derivatives thereof, such as quinolinyl, quinazolinyl, isoquinolinyl, and the like; or azoctinyl, indolizinyl, purinyl and the like, and the benzo derivatives thereof; or cinnolinyl, phthalazinyl, quinazolinyl, quinoxalinyl, naphthyridinyl, pteridinyl, carbazolyl, acridinyl, phenazinyl, phenothiazinyl and phenoxazinyl, and the like. The term “5- to 6-membered heteroaryl” refers to an aromatic ring system having 5 or 6 ring atoms and containing 1, 2 or 3, preferably 1-2, heteroatoms independently selected from N, O and S, with the remaining ring atoms being C.

The term “heteroarylene” refers to a residue derived from a heteroaryl group further removing one hydrogen atom.

The term “halo” or “halogen” refers to fluorine, chlorine, bromine or iodine.

The term “therapeutically effective amount” refers to:
an amount of the compound of the present disclosure for (i) treating a particular disease, condition, or disorder, (ii) alleviating, ameliorating, or eliminating one or more symptoms of a specific disease, condition, or disorder, or (iii) delaying the onset of one or more symptoms of a specific disease, condition, or disorder described herein.

The amount of a compound of the present disclosure that constitutes a “therapeutically effective amount” varies depending on the compound, the disease state and its severity, the administration regimen, and the age of the mammal to be treated, but can be determined routinely by those skilled in the art based on their knowledge and the content of the present disclosure.

The term “pharmaceutically acceptable” refers to those compounds, materials, compositions and/or dosage forms which are, within the scope of sound medical judgment, suitable for use in contact with tissues of humans and animals without excessive toxicity, irritation, allergic responses or other problems or complications, and commensurate with a reasonable benefit/risk ratio.

The term “pharmaceutically acceptable salt” refers to salts of pharmaceutically acceptable acids or bases, including salts formed from the compound and an inorganic or organic acid, and salts formed from the compound and an inorganic or organic base.

The term “pharmaceutical composition” refers to a mixture of one or more of the compounds or the salts thereof of the present disclosure and pharmaceutically acceptable excipients. The pharmaceutical composition is intended to facilitate administration of the compound of the present disclosure to an organism.

The term “pharmaceutically acceptable excipients” refers to those excipients which do not have a significant irritating effect on an organism and do not impair the biological activity and properties of the active compound. Suitable excipients are well known to those skilled in the art, such as carbohydrates, waxes, water-soluble and/or water-swellable polymers, hydrophilic or hydrophobic materials, gelatin, oil, solvents, water, and the like.

The words “comprise” and variations thereof such as “comprises” or “comprising”, may be understood in an open, non-exclusive sense, i.e., “including, but not limited to”.

The present disclosure also includes isotopically labeled compounds of the present disclosure that are identical to those described herein, but in which one or more atoms are replaced by atom(s) having an atomic weight or mass number different from those commonly found in nature. Examples of isotopes that can be incorporated to the compounds of the present disclosure include isotopes of hydrogen, carbon, nitrogen, oxygen, phosphorus, sulfur, fluorine, iodine, and chlorine, such as ²H, ³H, ¹¹C, ¹³C, ¹⁴C, ¹³N, ¹⁵N, ¹⁵O, ¹⁷O, ¹⁸O, ³¹P, ³²p, ³⁵S, ¹⁸F, ¹²³I, ¹²⁵I, and ³⁶Cl, respectively, and the like.

Certain isotopically labeled compounds of the present disclosure (e.g., labeled with ³H and ¹⁴C) can be used to analyze tissue distribution of compounds and/or substrates. Tritiated (i.e. ³H) and carbon-14 (i.e. ¹⁴C) isotopes are particularly preferred for their ease of preparation and detectability. Positron emitting isotopes such as ¹⁵O, ¹³N, ¹¹C, and ¹⁸F can be used in positron emission tomography (PET) studies to determine substrate occupancy. Isotopically labeled compounds of the present disclosure can generally be prepared by following procedures analogous to those disclosed in the schemes and/or examples below while substituting a non-isotopically labeled reagent with an isotopically labeled reagent.

The pharmaceutical composition of the present disclosure can be prepared by combining the compound of the present disclosure with suitable pharmaceutically acceptable excipients, for example, can be formulated into solid, semi-solid, liquid or gaseous formulations, such as tablets, pills, capsules, powders, granules, ointments, emulsions, suspensions, suppositories, injections, inhalants, gels, microspheres, aerosols, and the like.

Typical routes of administration of a compound of the present disclosure or a pharmaceutically acceptable salt thereof or a pharmaceutical composition thereof include, but are not limited to, oral, rectal, topical, inhalation, parenteral, sublingual, intravaginal, intranasal, intraocular, intraperitoneal, intramuscular, subcutaneous, and intravenous administration.

The pharmaceutical composition of the present disclosure can be manufactured by methods well known in the art, such as conventional methods of mixing, dissolving, granulating, emulsifying, lyophilizing, and the like.

In some embodiments, the pharmaceutical composition is in an oral form. For oral administration, the pharmaceutical composition may be formulated by mixing the active compound with pharmaceutically acceptable excipients well known in the art. These excipients enable the compound of the present disclosure to be formulated into tablets, pills, lozenges, dragees, capsules, liquids, gels, slurries, suspensions, and the like, for oral administration to a patient.

Solid oral compositions can be prepared by conventional mixing, filling or tableting methods. For example, it can be obtained by mixing the active compound with solid excipients, optionally grinding the resulting mixture, adding other suitable excipients if desired, and then processing the mixture into granules to obtain the cores of tablets or dragees. Suitable excipients include, but are not limited to, binders, diluents, disintegrants, lubricants, glidants or flavoring agents, and the like.

The pharmaceutical compositions may also be suitable for parenteral administration, such as sterile solutions, suspensions or lyophilized products in suitable unit dosage forms.

In all of the methods of administration of the compounds of general formula (I) described herein, the daily administration dose is from 0.01 mg/kg to 200 mg/kg body weight, in the form of a single dose or divided doses.

### DETAILED DESCRIPTION

The present disclosure will be described in detail below by way of examples and the accompanying drawings, but the following examples should not be regarded as limiting the scope of the present disclosure.

The structures of compounds are determined by nuclear magnetic resonance (NMR) and/or mass spectrometry (MS). The unit of NMR shift is 10⁻⁶ (ppm). The solvents for NMR determination are deuterated dimethyl sulfoxide, deuterated chloroform, deuterated methanol, etc., and the internal standard is tetramethylsilane (TMS); “IC₅₀” refers to the half maximal inhibitory concentration, which refers to the concentration at which half of the maximum inhibitory effect is achieved.

The eluent hereinafter can be composed of two or more solvents to form a mixed eluent, and the ratio thereof is the volume ratio of each solvent. For example, “petroleum ether:ethyl acetate=5:1” means that the volume ratio of petroleum ether to ethyl acetate in the mixed eluent during the elution process is 5:1.

Unless otherwise stated, % refers to wt %.

### Abbreviations

DCM: dichloromethane; Pyridine: Pyridine; CDI: N,N′-carbonyldiimidazole; THF: tetrahydrofuran; LAH: lithium aluminum tetrahydride; EtOH: ethanol; DMF: N,N-dimethylformamide; EDCI: 1-ethyl-(3-dimethylaminopropyl) carbodiimide hydrochloride; HOBt: 1-hydroxybenzotriazole; TEA: triethylamine; NH₂OH·HCl: hydroxylamine hydrochloride; Pd(OAc)₂: palladium acetate; Cs₂CO₃: cesium carbonate; Butyldi-1-adamantylphosphine: n-butylbis (1-adamantyl) phosphine; N-boc-Methyltrifluoroborate: potassium N-aminomethyltrifluoroborate; dioxane: 1, 4-dioxane; Hydroxymethyl tributylstannane: (tributyltin) methanol; Pd(Ph₃)₄: tetrakis (triphenylphosphine) palladium; Triphosgene: Triphosgene; DMF-DMA: N,N-dimethylformamide dimethyl acetal; BH₃Me₂S: borane dimethyl sulfide; Propiolic Acid: Propynoic acid; DMSO: dimethyl sulfoxide; DBU: 1, 8-diazabicyclo [5.4.0] undec-7-ene; tert-Butyl bromoacetate: tert-Butyl bromoacetate; HATU: 2-(7-azabenzotriazole)-N, N, N′, N′-tetramethyluronium hexafluorophosphate; DIEA: N,N-diisopropylethylamine; DMAP: 4-dimethylaminopyridine; (Boc)₂O: di-tert-butyl dicarbonate; TFA: trifluoroacetic acid; Pd₂(dba)₃: tris (dibenzylideneacetone) dipalladium; TBA: tert-butyl acrylate; NN-Dicyclohexylmethylamine:NN-Dicyclohexylmethylamine; Tri-tert-butylphosphinetetrafluoroborate: Tri-tert-butylphosphine tetrafluoroborate.

### Intermediate 1: (4-nitrophenyl)-N-(3-chloro-4-methyl-phenyl) carbamate

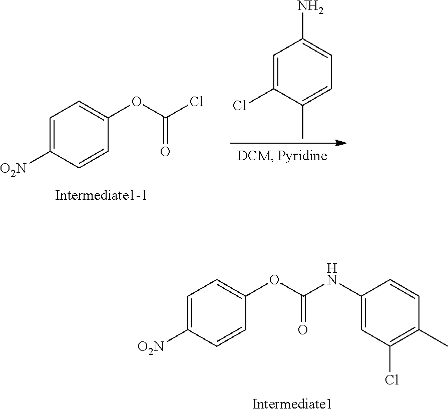

Intermediate 1-1 (500 mg, 2.48 mmol) and 3-chloro-p-toluidine (386 mg, 2.73 mmol) were placed into a reaction tube, the air in the reaction tube was replaced with argon, and the reaction tube was placed in a water bath to cool down. Dichloromethane (2 mL) and pyridine (392 mg, 4.96 mmol) were added into the reaction tube, the reaction solution was naturally heated to room temperature and stirred for one hour. After the reaction was complete, the reaction solution was concentrated to obtain a crude product, which was further separated by normal phase chromatography column (petroleum ether:ethyl acetate=5:1) to obtain intermediate 1 (532 mg, 70% yield). M/z (ESI): 307 [M+H]⁺.

### Intermediate 2: tert-butyl (4-amino-2-chlorophenyl) (methyl) carbamate

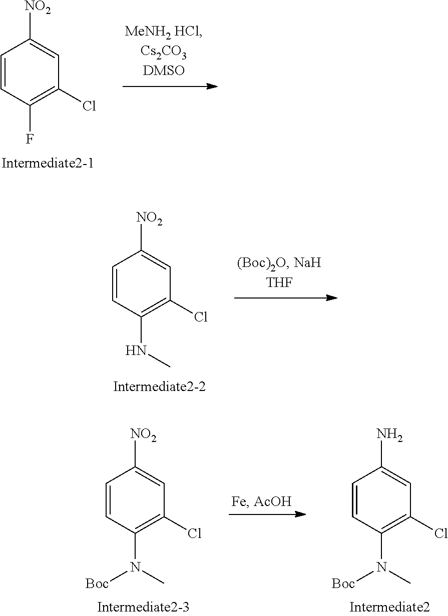 [SMILES: O=[N+]([O-])c1ccc(F)c(Cl)c1 ; CNc1ccc([N+](=O)[O-])cc1Cl ; CN(C(=O)OC(C)(C)C)c1ccc([N+](=O)[O-])cc1Cl ; CN(C(=O)OC(C)(C)C)c1ccc(N)cc1Cl]

### Step 1: 2-Chloro-N-methyl-4-nitroaniline (Intermediate 2-2)

Intermediate 2-1 (500 mg, 2.85 mmol), methylamine hydrochloride (384 mg, 5.70 mmol) and cesium carbonate (1.86 g, 5.70 mmol) were placed in a reaction flask, dimethyl sulfoxide (5 mL) was added, and the reaction solution was stirred in an oil bath at 100° C. for 1 h. After the reaction was completed, the reaction solution was purified by reverse phase column chromatography (eluent water:acetonitrile=1:1, 0.1% formic acid) to obtain intermediate 2-2 (500 mg, 94% yield). M/z (ESI): 187 [M+H]⁺.

### Step 2: tert-butyl(2-chloro-4-nitrophenyl) (methyl) carbamate (Intermediate 2-3)

Intermediate 2-2 (500 mg, 2.68 mmol), sodium hydride (128 mg, 3.22 mmol, purity 60%) were placed in a reaction tube, the air in the reaction tube was displaced with argon, and tetrahydrofuran (5 mL) was added at 0° C. and stirred for 0.5 h. Then di-tert-butyl dicarbonate (877 mg, 4.02 mmol) was added, and the reaction was placed in an oil bath at 50° C. and stirred for 5 h. After the reaction was completed, the reaction solution was concentrated and purified by reversed phase column chromatography (eluent was water:acetonitrile=1:1) to obtain intermediates 2-3 (500 mg, 65% yield). M/z (ESI): 287 [M+H]⁺.

### Step 3: tert-butyl (4-amino-2-chlorophenyl) (methyl) carbamate (Intermediate 2)

Intermediate 2-3 (500 mg, 1.74 mmol) was dissolved in acetic acid (5 mL), iron powder (292 mg, 5.23 mmol) was added, and the reaction was stirred at room temperature for 1 hour. After the reaction was completed, the reaction solution was concentrated and purified by reversed phase column chromatography (eluent was water:acetonitrile=1:1) to obtain Intermediate 2 (250 mg, 55% yield). M/z (ESI): 257 [M+H]⁺.

### Example 1: 3-(3-oxo-1H-imidazo [1,5-a] indol-2 (3H)-yl) piperidine-2, 6-dione

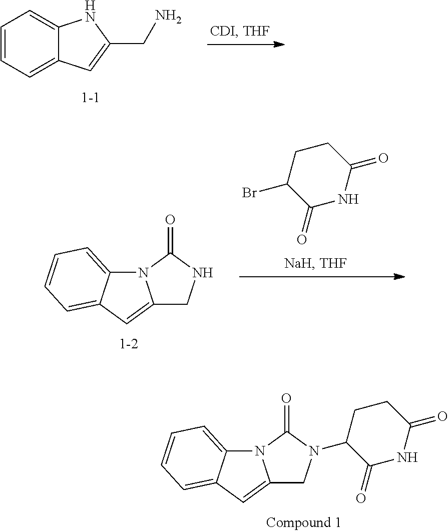 [SMILES: NCc1cc2ccccc2[nH]1 ; O=C1NCc2cc3ccccc3n21 ; O=C1CCC(Br)C(=O)N1 ; O=C1CCC(N2Cc3cc4ccccc4n3C2=O)C(=O)N1]

### Step 1: 1, 2-dihydro-3H-imidazo [1,5-a] indol-3-one (Compound 1-2)

Compound 1-1 (400 mg, 2.73 mmol) and N, N′-carbonyldiimidazole (488 mg, 3.01 mmol) were placed in a dry sealed tube, and tetrahydrofuran (3 mL) was added and the sealed tube was sealed. The reaction solution was stirred in an oil bath at 90° C. for 12 h. After the reaction was completed, the reaction solution was concentrated to obtain a crude product purified by reverse phase column chromatography (eluent was water:acetonitrile=1:1, 0.1% formic acid) to obtain compound 1-2 (170 mg, 36% yield). M/z (ESI): 173 [M+H]⁺.

### Step 2: 3-(3-oxo-1H-imidazo [1,5-a] indol-2 (3H)-yl) piperidine-2, 6-dione (Compound 1)

Compound 1-2 (80 mg, 0.46 mmol) was dissolved in tetrahydrofuran (2 mL) under nitrogen atmosphere and cooled to 0° C. Sodium hydride (55.7 mg, 2.32 mmol) was added, and was continued to stirred at this temperature for 0.5 h. 3-bromopiperidine-2, 6-dione (107.0 mg, 0.55 mmol) dissolved in tetrahydrofuran solution (2 mL) was added dropwise to the reaction solution, after adding, the reaction solution was continued to react at this temperature for 10 min, and then stirred in an oil bath at 60° C. for 4 h. After the reaction was completed, the reaction solution was cooled to 0° C., and the acetic acid (105.0 mg, 1.75 mmol) and saturated ammonium chloride solution (5 mL) were sequentially added to quench the reaction solution. The reaction solution was concentrated to obtain a crude product, which was purified by reversed phase column chromatography (eluent was water:acetonitrile=1:1, containing 0.1% formic acid) to obtain compound 1 (17.5 mg, yield 13%). M/z (ESI): 284 [M+H]⁺.

¹H NMR (400 MHz, DMSO-d6) δ 11.04 (s, 1H), 7.84 (d, J=7.8 Hz, 1H), 7.64 (d, J=7.6 Hz, 1H), 7.25 (dt, J=15.6, 7.4 Hz, 2H), 6.52 (s, 1H), 4.96 (dd, J=13.5, 5.1 Hz, 1H), 4.59 (d, J=16.5 Hz, 1H), 4.42 (d, J=16.4 Hz, 1H), 2.97-2.86 (m, 1H), 2.61 (d, J=17.4 Hz, 1H), 2.43-2.30 (m, 1H).

### Example 2: 1-(3-chloro-4-methylphenyl)-3-((2-(2, 6-dioxopiperidin-3-yl)-3-oxo-2, 3-dihydro-1H-imidazo [1, 5-a] indol-7-yl) methyl) urea

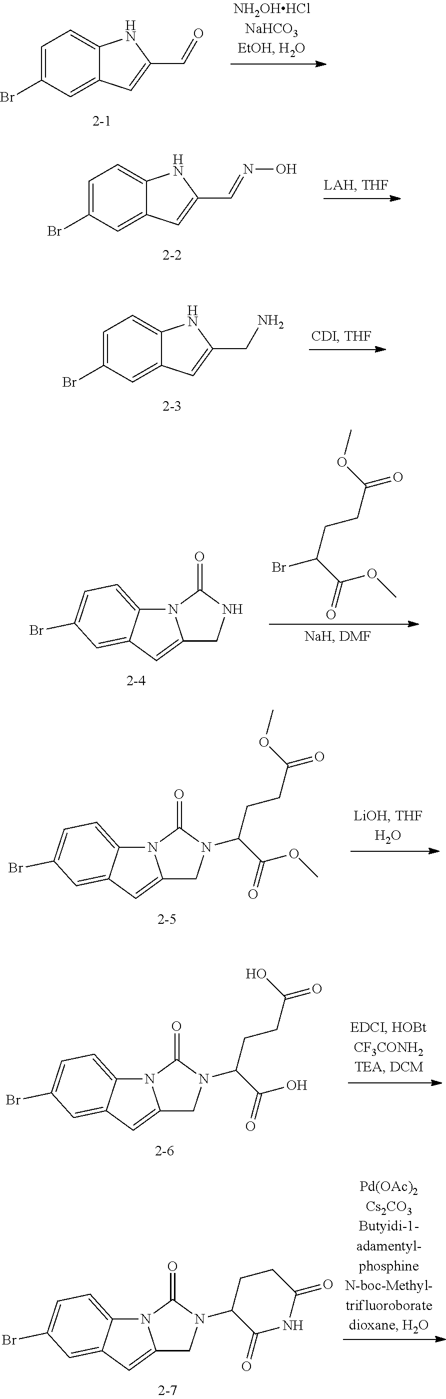

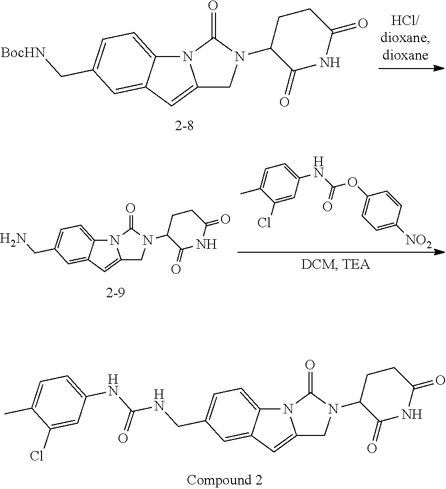

### Step 1: 5-Bromo-1H-indole-2-carboxaldehyde oxime (Compound 2-2)

5-Bromo-1H-indole-2-carbaldehyde (1000 mg, 4.46 mmol), hydroxylamine hydrochloride (465 mg, 6.69 mmol), sodium bicarbonate (749 mg, 8.92 mmol) were placed in a flask, ethanol (15 mL) and water (10 mL) were added, and the reaction solution was stirred in an oil bath at 70° C. for 3 h. After the reaction was completed, the reaction solution was concentrated, extracted with ethyl acetate and water, and the organic phase was dried and concentrated to obtain compound 2-2 (1010 mg, 95% yield). M/z (EST): 239 [M+H]⁺.

### Step 2: (5-bromo-1H-indol-2-yl) methylamine (Compound 2-3)

Compound 2-2 (1010 mg, 4.22 mmol) was placed in a flask, anhydrous tetrahydrofuran (25 mL) was added, and the reaction solution was placed in an ice bath at 0° C. and stirred to cool down. After dropping to 0° C., lithium aluminum tetrahydride (240 mg, 6.33 mmol) was slowly added, then the reaction solution was placed in an oil bath at 70° C. and stirred for 2 h. After the reaction was completed, the reaction solution was stirred in an ice bath at 0° C. to cool down, sodium sulfate decahydrate was slowly added to quench the reaction. After quenching, the reaction solution was filtered, and the filtrate was extracted with ethyl acetate and water, and the organic phase reaction solution was concentrated to obtain a crude product, which was purified by reverse phase column chromatography (eluent was water:acetonitrile=1:1) to obtain compound 2-3 (694 mg, yield 73%). M/z (ESI): 225 [M+H]⁺.

### Step 3: 7-bromo-1, 2-dihydro-3H-imidazo [1, 5-a] indol-3-one (Compound 2-4)

Compound 2-3 (694 mg, 3.08 mmol) was placed in a dry sealed tube, the air in the reaction tube was displaced with argon gas, and tetrahydrofuran (3 mL) was added and the dry sealed tube was sealed. The reaction solution was placed in an ethanol-dry ice bath, stirred and cooled down, and N, N′-carbonyldiimidazole (550 mg, 3.39 mmol) dissolved in tetrahydrofuran (3 mL) was slowly added dropwise to the reaction solution. After the reaction was completed, the reaction solution was concentrated to obtain a crude product, which was purified by normal phase column chromatography (eluent was petroleum ether:ethyl acetate=5:1) to obtain compound 2-4 (526 mg, yield 68%). M/z (ESI): 251 [M+H]⁺.

### Step 4: Dimethyl 2-(7-bromo-3-oxo-1H-imidazo [1,5-a] indol-2 (3H)-yl) glutarate (Compound 2-5)

Compound 2-4 (526 mg, 2.10 mmol) and sodium hydride (75.2 mg, 3.14 mmol) were placed in a dry reaction tube, the air in the reaction tube was replaced with argon The reaction tube was placed in an ethanol-dry ice bath to cool down, and N, N-dimethylformamide (2 mL) was added, and the reaction solution was naturally warmed to room temperature and stirred for half an hour, and then stirred in an ice-water bath. Dimethyl 2-bromoglutarate (749 mg, 3.14 mmol) was added dropwise to the reaction solution, and continued stirring in an ice-water bath for half an hour and then stirred at room temperature for one hour. After the reaction was completed, acetic acid (250 mg, 4.2 mmol) was added to quench the reaction, and the reaction solution was purified by reverse phase column chromatography (eluent was water:acetonitrile=1:1) to obtain compound 2-5 (686 mg, yield 80%). M/z (ESI): 409 [M+H]⁺.

### Step 5: 2-(7-bromo-3-oxo-1H-imidazo [1,5-a] indol-2 (3H)-yl) glutaric acid (Compound 2-6)

Compound 2-5 (686 mg, 1.68 mmol) and lithium hydroxide (161 mg, 6.72 mmol) were placed in a flask, tetrahydrofuran (4 mL) and water (2 mL) were added, and stirred at room temperature for 3-5 hours. After the reaction was completed, hydrochloric acid (3N) was added to adjust the pH value of the reaction solution to 3-5, the reaction solution was concentrated, and then purified by reversed phase column chromatography (eluent was water:acetonitrile=1:1) to obtain compound 2-6 (170 mg, yield: 90%). M/z (ESI): 381 [M+H]⁺.

### Step 6: 3-(7-bromo-3-oxo-1H-imidazo [1,5-a] indol-2 (3H)-yl) piperidine-2,6-dione (Compound 2-7)

Compound 2-6 (575 mg, 1.51 mmol), trifluoroacetamide (255 mg, 2.26 mmol), 1-hydroxybenzotriazole (448 mg, 3.32 mmol) and 1-ethyl-(3-dimethylaminopropyl) carbodiimide hydrochloride (1012 mg, 5.28 mmol) were placed in a dry reaction tube, the air in the reaction tube was replaced with argon. The reaction tube was placed in an ethanol-dry ice bath to cool down. Methylene chloride (10 mL) and triethylamine (687 mg, 6.80 mmol) were added to the reaction tube, and the reaction solution was naturally warmed to room temperature and stirred for half an hour, and then placed in an oil bath at 35° C. and stirred for 5 hours. After the reaction was completed, the reaction solution was concentrated and purified by reverse phase column chromatography (eluent was water:acetonitrile=1:1) to obtain compound 2-7 (448 mg, yield: 82%). M/z (ESI): 362 [M+H]⁺.

### Step 7: tert-butyl ((2-(2, 6-dioxopiperidin-3-yl)-3-oxo-2, 3-dihydro-1H-imidazo [1,5-a] indol-7-yl) methyl) carbamate (Compound 2-8)

Compound 2-7 (448 mg, 1.24 mmol), palladium acetate (20.6 mg, 0.09 mmol), n-butylbis (1-adamantyl) phosphine (88.9 mg), (((tert-butoxycarbonyl) amino) methyl) trifluoroborate potassium salt (382 mg, 1.61 mmol) and cesium carbonate (808 mg, 2.48 mmol) were placed in a reaction tube, the air in the reaction tube was replaced with argon, and 1, 4-dioxane (3 mL) and water (0.3 mL) were added to the reaction tube, and then the reaction was placed in an oil bath at 100° C. and stirred for 5 to 8 hours. After the reaction was completed, the reaction solution was purified by reverse phase column chromatography (eluent was water:acetonitrile=1:1) to obtain compound 2-8 (296 mg, 58% yield). M/z (EST): 413 [M+H]⁺.

### Step 8: 3-(7-(aminomethyl)-3-oxo-1H-imidazo [1,5-a] indol-2 (3H)-yl) piperidine-2, 6-dione (Compound 2-9)

Compound 2-8 (296 mg, 0.72 mmol) was placed in a reaction tube, the air in the reaction tube was displaced with argon, and 1, 4-dioxane (2 mL) and 1, 4-dioxane-hydrogen chloride (4M)(2 mL) were added to the reaction tube, and the reaction tube was placed in an oil bath at 25° C. and stirred for 5 h. After the reaction was completed, the reaction solution solvent was removed by spin-dry to obtain crude compound 2-9 (245 mg, 98% yield). M/z (ESI): 313 [M+H]⁺.

### Step 9: 1-(3-chloro-4-methylphenyl)-3-((2-(2, 6-dioxopiperidin-3-yl)-3-oxo-2, 3-dihydro-1H-imidazo [1,5-a] indol-7-yl) methyl) urea (Compound 2)

Compound 2-9 (245 mg, 0.78 mmol) and (4-nitrophenyl)-N-(3-chloro-4-methyl-phenyl) carbamate (260 mg, 0.85 mmol) were placed in a dry reaction tube, the air in the reaction tube was displaced with argon, and dichloromethane (3 mL) and triethylamine (179 mg, 1.77 mmol) were added to the reaction tube, and the reaction tube was placed in an oil bath at 25° C. and stirred for 4 hours. After the reaction was completed, the reaction solution was concentrated to obtain a crude product, which was purified by reverse phase column chromatography (eluent was water:acetonitrile=1:1) to obtain compound 2 (179 mg, 53% yield). M/z (ESI): 480 [M+H]⁺.

¹H NMR (400 MHz, DMSO-d6) δ 11.06 (s, 1H), 8.76 (d, J=3.8 Hz, 1H), 7.87-7.76 (m, 1H), 7.68 (s, 1H), 7.56 (s, 1H), 7.24 (d, J=8.3 Hz, 1H), 7.15 (q, J=8.6 Hz, 2H), 6.80 (s, 1H), 6.52 (s, 1H), 4.96 (d, J=13.0 Hz, 1H), 4.59 (d, J=16.0 Hz, 1H)

### Example 3: (2-(2, 6-dioxopiperidin-3-yl)-3-oxo-2, 3-dihydro-1H-imidazo [1,5-a] indol-7-yl) methyl-(3-chloro-4-methylphenyl) carbamate (Compound 3)

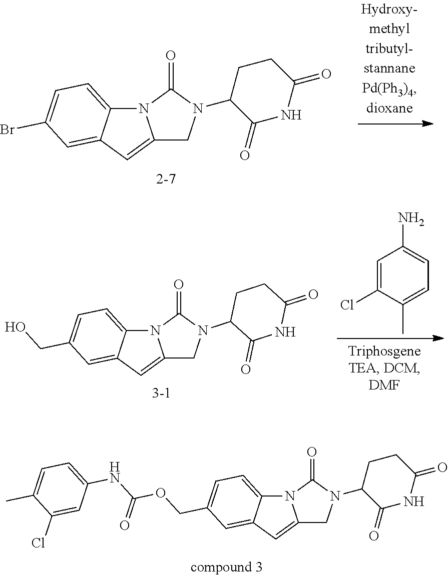

### Step 1: 3-(7-(hydroxymethyl)-3-oxo-1H-imidazo [1,5-a] indol-2 (3H)-yl) piperidine-2, 6-dione (Compound 3-1)

Compound 2-7 (27 mg, 0.074 mmol), (tributyltin) methanol (36 mg, 0.112 mmol) and tetrakis (triphenylphosphine) palladium (8 mg, 0.007 mmol) were added to 1, 4-dioxane (1 mL) under nitrogen atmosphere and reacted at 80° C. for 16 h. After the reaction was completed, the reaction solution was concentrated to obtain a crude product, which was purified by reverse phase column chromatography (eluent was water:acetonitrile=1:1) to obtain compound 3-1 (6 mg, yield: 28%). M/z (ESI): 314 [M+H]⁺.

### Step 2: (2-(2, 6-dioxopiperidin-3-yl)-3-oxo-2, 3-dihydro-1H-imidazo [1,5-a] indol-7-yl) methyl-(3-chloro-4-methylphenyl) carbamate (Compound 3)

3-chloro-p-toluidine (10 mg, 0.07 mmol) and triethylamine (15 mg, 0.14 mmol) were dissolved in dichloromethane (1 mL) under nitrogen atmosphere, and triphosgene (8 mg, 0.027 mmol) dissolved in dichloromethane (1 mL) was added to the reaction solution at 0° C. The reaction solution was warmed to room temperature, and stirred for 30 minutes, then concentrated, dissolved in N, N-dimethylformamide (1 mL), replaced with nitrogen three times, and then compound 3-1 (18 mg, 0.057 mmol) was added to the above solution, and the reaction was stirred at room temperature for 30 minutes. After the reaction was completed, the reaction solution was concentrated to obtain a crude product, which was purified by reverse phase column chromatography (eluent was water:acetonitrile=1:1) to obtain compound 3 (11 mg, yield: 30%). M/z (ESI): 481 [M+H]⁺.

¹H NMR (400 MHz, DMSO-d6) δ 11.06 (s, 1H), 9.88 (s, 1H), 7.91-7.85 (m, 1H), 7.73 (s, 1H), 7.61 (s, 1H), 7.37 (d, J=8.4 Hz, 1H), 7.33-7.22 (m, 2H), 6.57 (s, 1H), 5.25 (s, 2H), 4.97 (d, J=13.3 Hz, 1H), 4.61 (d, J=16.7 Hz, 1H)

### Example 4: N-((2-(2, 6-dioxopiperidin-3-yl)-3-oxo-2, 3-dihydro-1H-imidazo [1, 5-a] indol-7-yl) methyl)-3-(4-methyl-3-(methylamino) phenyl) propinamide

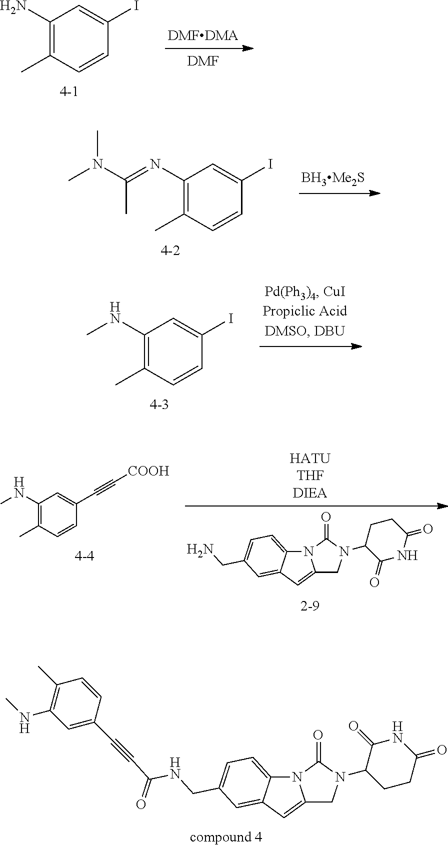

### Step 1: (E)-N′-(5-iodo-2-methylphenyl)-N, N-dimethylacetamidine (Compound 4-2)

Compound 4-1 (233 mg, 1 mmol), N, N-dimethylformamide dimethyl acetal (600 mg, 5 mmol) were added to a flask, and N, N-dimethylformamide (2 mL) was added and stirred in an oil bath at 100° C. for 3 h. After the reaction was completed, the reaction solution was concentrated, extracted with ethyl acetate and water, and the organic phase was dried and concentrated to obtain crude compound 4-2 (296 mg, 97% yield). M/z (ESI): 303 [M+H]⁺.

### Step 2: N, 2-dimethyl-5-iodoaniline (Compound 4-3)

Compound 4-2 (296 mg, 0.97 mmol) was placed in a flask, anhydrous tetrahydrofuran (1 mL) was added, and argon was displaced three times under vacuum. Borane dimethyl sulfide solution (1.5 mL, 2M) was added with a syringe, and then the reaction solution was stirred at room temperature for 2 hours. After the reaction was completed, methanol was slowly added to quench. After quenching, 1 M hydrochloric acid was added and stirred for 30 minutes. The organic phase reaction solution was concentrated and purified by reversed-phase column chromatography (eluent was water:acetonitrile=1:1) to obtain compound 4-3 (152 mg, 61% yield). M/z (ESI): 248 [M+H]⁺.

### Step 3: 3-(4-methyl-3-(methylamino) phenyl) propynoic acid (Compound 4-4)

Compound 4-3 (152 mg, 0.60 mmol), cuprous iodide (17 mg, 0.9 mmol), tetrakis (triphenylphosphine) palladium (17 mg, 0.015 mmol) were placed in a dry sealed tube, the air in the reaction tube was replaced with argon, and dimethyl sulfoxide (2 mL) was added and the dry sealed tube was sealed, and 1, 8-diazabicyclo [5.4. 0]undec-7-ene (273 mg, 1.8 mmol) and propynoic acid (63 mg, 0.9 mmol) were added to the reaction solution with a syringe, and the reaction solution was warmed to 70° C. and stirred for 1 h. After the reaction was completed, the reaction solution was purified by normal phase column chromatography (eluent: petroleum ether:ethyl acetate=1:1) to obtain compound 4-4 (75 mg, 60% yield). M/z (ESI): 190 [M+H]⁺.

### Step 4: N-((2-(2, 6-dioxopiperidin-3-yl)-3-oxo-2, 3-dihydro-1H-imidazo [1,5-a] indol-7-yl) methyl)-3-(4-methyl-3-(methylamino) phenyl) propanamide (Compound 4)

Compound 4-4 (10 mg, 0.05 mmol), compound 2-9 (18 mg, 0.06 mmol) and 2-(7-azabenzotriazole)-N, N, N′, N′-tetramethyluronium hexafluorophosphate (22 mg, 0.06 mmol) were placed in a dry reaction tube, tetrahydrofuran (0.5 mL) and N, N-diisopropylethylamine (15 mg, 0.11 mmol) were added and stirred at room temperature for one hour. After the reaction was completed, the reaction solution was purified by reverse phase column chromatography (eluent: water:acetonitrile=1:1) to obtain compound 4 (5.58 mg, 24% yield).

M/z (ESI): 484 [M+H]⁺

¹H NMR (400 MHz, DMSO-d6) δ 11.06 (s, 1H), 9.27 (t, J=6.1 Hz, 1H), 8.38 (s, 1H), 7.81 (d, J=8.3 Hz, 1H), 7.55 (s, 1H), 7.22 (dd, J=8.4, 1.6 Hz, 1H), 7.02 (d, J=7.6 Hz, 1H), 6.72 (dd, J=7.5, 1.5 Hz, 1H), 6.61-6.51 (m, 2H), 5.32 (q, J=5.0 Hz, 1H), 4.97 (dd, J=13.4, 5.1 Hz, 1H), 4.60 (dd, J=16.5, 1.7 Hz, 1H), 4.44 (d, J=5.6 Hz, 2H)

### Example 5: 2-(2, 6-dioxopiperidin-3-yl)-3-oxo-2, 3-dihydro-1H-imidazo [1,5-a] indol-7-yl) methyl (2-fluoro-5-trifluoromethoxy) phenyl) carbamate

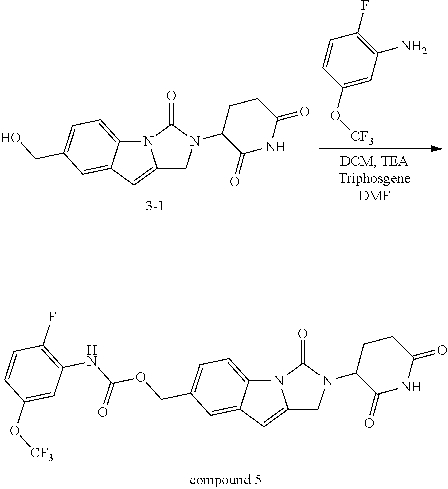

2-fluoro-3-(trifluoromethoxy) aniline (12.5 mg, 0.064 mmol) was placed in a dry reaction tube, the air in the reaction tube was replaced with argon, and dichloromethane (2 mL) and triethylamine (13 mg, 0.128 mmol) were added to the reaction tube, and the reaction tube was placed in an ice-water bath at 0° C. and cooled down. Triphosgene (7.6 mg, 0.025 mmol) was dissolved in dichloromethane (1 mL), and the triphosgene solution was slowly added dropwise into the reaction solution, which was naturally warmed to room temperature and reacted for 0.5 hours, then the solvent was removed by spin-dry. Compound 3-1 (10.0 mg, 0.032 mmol) dissolved in N, N-dimethylformamide (1 mL), was added into a reaction tube and the reaction solution was reacted at room temperature for 2 to 3 hours, and then purified by reversed-phase column chromatography (The eluent was water:acetonitrile=1:1) to obtain compound 5 (6.0 mg, yield 35%). M/z (ESI): 535 [M+H]⁺.

¹H NMR (400 MHz, DMSO-d6) δ 11.06 (s, 1H), 9.84 (s, 1H), 7.88-7.83 (m, 2H), 7.74 (s, 1H), 7.42-7.32 (m, 2H), 7.17-7.09 (m, 1H), 6.57 (s, 1H), 5.28 (s, 2H), 4.97 (dd, J=13.4, 5.1 Hz, 1H), 4.61 (dd, J=16.5, 1.7 Hz, 1H), 4.44 (dd, J=16.5, 1.8 Hz, 1H), 2.99-2.85 (m, 1H), 2.70-2.57 (m, 1H), 2.44-2.31 (m, 1H).

### Example 6: (2-(2, 6-dioxopiperidin-3-yl)-3-oxo-2, 3-dihydro-1H-imidazo [1,5-a] indol-8-yl)-methyl-(3-chloro-4-(2-(2-(methylamino) ethoxy) ethyl) phenyl) carbamate

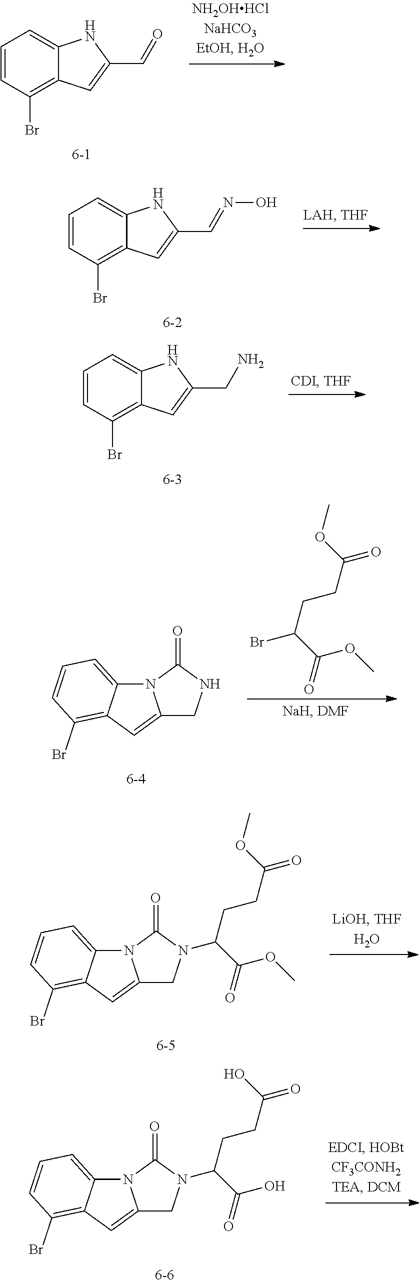

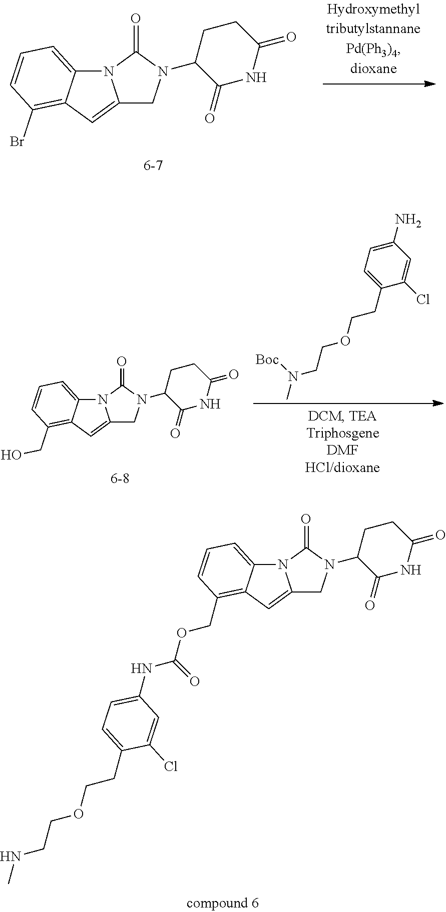 [SMILES: O=C1CCC(N2Cc3cc4c(Br)cccc4n3C2=O)C(=O)N1 ; O=C1CCC(N2Cc3cc4c(CO)cccc4n3C2=O)C(=O)N1 ; CN(CCOCCc1ccc(N)cc1Cl)C(=O)OC(C)(C)C ; CNCCOCCc1ccc(NC(=O)OCc2cccc3c2cc2n3C(=O)N(C3CCC(=O)NC3=O)C2)cc1Cl]

### Step 1: (E)-4-bromo-1H-indole-2-carboxaldehyde oxime (Compound 6-2)

Compound 6-1 (1000 mg, 4.46 mmol), hydroxylamine hydrochloride (465 mg, 6.69 mmol), sodium bicarbonate (749 mg, 8.92 nmol) were placed in a flask, ethanol (15 mL) and water (10 mL) were added, and the reaction solution was stirred in an oil bath at 70° C. for 3 h. After the reaction was completed, the reaction solution was concentrated, extracted with ethyl acetate and water, and the organic phase was dried and concentrated to obtain crude compound 6-2 (1010 mg, 95% yield). M/z (ESI): 239 [M+H]⁺.

### Step 2: (4-bromo-1H-indol-2-yl) methylamine (Compound 6-3)

Compound 6-2 (1010 mg, 4.22 mmol) was placed in a flask, anhydrous tetrahydrofuran (25 mL) was added, and the reaction solution was placed in an ice bath at 0° C., stirred and cooled down. After lowering the temperature, lithium aluminum tetrahydride (240 mg, 6.33 mmol) was slowly added, and then the reaction solution was placed in an oil bath at 70° C. and stirred for 2 h. After the reaction was completed, the reaction solution was stirred in an ice bath at 0° C. to cool down, and sodium sulfate decahydrate was slowly added to quench. After quenching, the reaction solution was filtered, and the filtrate was extracted with ethyl acetate and water, and the organic phase reaction solution was concentrated to obtain a crude product, which was purified by reversed phase column chromatography (eluent was water:acetonitrile=1:1) to obtain compound 6-3 (694 mg, yield 73%). M/z (ESI): 225 [M+H]⁺.

### Step 3: 8-bromo-1, 2-dihydro-3H-imidazo [1,5-a] indol-3-one (Compound 6-4)

Compound 6-3 (694 mg, 3.08 mmol) was placed in a dry sealed tube, the air in the reaction tube was displaced with argon, tetrahydrofuran (3 mL) was added and then the dry sealed tube was sealed. The reaction solution was stirred in an ethanol-dry ice bath to cool down. N, N′-carbonyldiimidazole (550 mg, 3.39 mmol) was placed in a dry flask, and then tetrahydrofuran (3 mL) was added to dissolve it, and N, N′-carbonyldiimidazole solution was slowly added dropwise to the reaction solution, then the reaction solution was naturally warmed to room temperature and stirred for 1 h. After the reaction was completed, the reaction solution was concentrated to obtain a crude product, which was purified by normal phase column chromatography (eluent was petroleum ether:ethyl acetate=1:1) to obtain compound 6-4 (526 mg, yield 68%). M/z (ESI): 251 [M+H]⁺.

### Step 4: Dimethyl 2-(8-bromo-3-oxo-1H-imidazo [1,5-a] indol-2 (3H)-yl) glutarate (Compound 6-5)

Compound 6-4 (526 mg, 2.10 mmol) and sodium hydride (75.2 mg, 3.14 mmol) were placed in a dry reaction tube, the air in the reaction tube was replaced with argon, the reaction tube was placed in an ethanol-dry ice bath to cool down. N, N-dimethylformamide (2 mL) was added to the reaction tube, then the reaction solution was naturally warmed to room temperature and stirred for half an hour, and then stirred in an ice-water bath. Dimethyl 2-bromoglutarate (749 mg, 3.14 mmol) was added dropwise to the reaction solution, and continued stirring in an ice-water bath for half an hour and then stirred at room temperature for one hour. After the reaction was completed, acetic acid (250 mg, 4.2 mmol) was added to quench the reaction, and the reaction solution was purified by reverse phase column chromatography (eluent was water:acetonitrile=1:1) to obtain compound 6-5 (686 mg, yield 80%). M/z (ESI): 409 [M+H]⁺.

### Step 5: 2-(8-bromo-3-oxo-1H-imidazo [1, 5-a] indol-2 (3H)-yl) glutaric acid (Compound 6-6)

Compound 6-5 (686 mg, 1.68 mmol) and lithium hydroxide (161 mg, 6.72 mmol) were placed in a flask, tetrahydrofuran (4 mL) and water (2 mL) were added, and stirred at room temperature for 3-5 hours. After the reaction was completed, hydrochloric acid (3N) was added to adjust the pH value of the reaction solution to 3-5, the reaction solution was concentrated, and then purified by reversed-phase column chromatography (eluent was water:acetonitrile=1:1) to obtain compound 6-6 (170 mg, yield: 90%). M/z (ESI): 381 [M+H]⁺.

### Step 6: 3-(8-bromo-3-oxo-1H-imidazo [1,5-a] indol-2(3H)-yl)-piperidine-2,6-dione (Compound 6-7)

Compound 6-6 (575 mg, 1.51 mmol), trifluoroacetamide (255 mg, 2.26 mmol), 1-hydroxybenzotriazole (448 mg, 3.32 mmol) and 1-ethyl-(3-dimethylaminopropyl) carbondiimide hydrochloride (1012 mg, 5.28 mmol) were placed into a dry reaction tube, the air in the reaction tube was displaced with argon, and the reaction tube was placed in an ethanol-dry ice bath to cool down. After the reaction was completed, the reaction solution was concentrated and purified by reverse phase column chromatography (eluent was water:acetonitrile=1:1) to obtain compound 6-7 (448 mg, yield: 82%). M/z (ESI): 362 [M+H]⁺.

### Step 7: 3-(8-(hydroxymethyl)-3-oxo-1H-imidazo [1,5-a] indol-2 (3H)-yl) piperidine-2, 6-dione (Compound 6-8)

Compound 6-7 (270 mg, 0.74 mmol) and tetrakis (triphenylphosphine) palladium (86 mg, 0.07 mmol) were placed in a reaction tube, the air in the reaction tube was replaced with argon, 1, 4-dioxane (6 mL) and tributyltin methanol (360 mg, 1.1 mmol) were added to the reaction tube, and the reaction tube was placed in an oil bath at 80° C. and stirred for 15-20 hours. After the reaction was completed, the reaction solution was purified by reverse phase column chromatography (eluent was water:acetonitrile=1:1) to obtain compound 6-8 (150 mg, 64% yield). M/z (EST): 314 [M+H]⁺.

### Step 8: (2-(2, 6-dioxopiperidin-3-yl)-3-oxo-2, 3-dihydro-1H-imidazo [1, 5-a] indol-8-yl)-methyl-(3-chloro-4-(2-(2-(methylamino) ethoxy) ethyl) phenyl) carbamate (Compound 6)

Tert-butyl N-[2-[2-(4-amino-2-chloro-phenyl) ethoxy]ethyl]-N-methyl-carbamate (21.0 mg, 0.064 mmol) was placed in a dry reaction tube, the air in the reaction tube was replaced with argon, and dichloromethane (2 mL) and triethylamine (13.0 mg, 0.127 mmol) were added to the reaction tube, which was placed in an ice-water bath at 0° C. and stirred. After cooling down, triphosgene (7.6 mg, 0.025 mmol) was dissolved in dichloromethane (1 mL) and the triphosgene solution was slowly added dropwise into the reaction solution, which was naturally warmed to room temperature for 0.5 hours, then the solvent was removed by spin-dry. Compound 6-8 (10 mg, 0.032 mmol) dissolved in N, N-dimethylformamide (1 mL) was added into a reaction tube, and the reaction solution was reacted at room temperature for 2-3 hours, and then purified by reversed-phase column chromatography (eluent is water:acetonitrile=1:1) to obtain the intermediate. The intermediate was placed in a dry reaction tube, the air in the reaction tube was replaced with argon, tetrahydrofuran (0.5 mL) was added into the reaction tube, the reaction tube was cooled to 0° C. with an ice bath and stirred, then dioxane hydrogen chloride solution (4M, 1.5 mL) was slowly added dropwise, and the reaction was stirred in the ice bath for 1 to 2 hours, and detected by LC-MS. After the reaction was completed, the reaction solution was purified by reverse phase column chromatography (eluent was water:acetonitrile=1:1) to obtain compound 6 (6.0 mg, 31% yield). M/z (EST): 568 [M+H]⁺.

¹H NMR (400 MHz, DMSO-d6) δ 9.90 (s, 1H), 8.33 (s, 1H), 7.86 (dd, J=7.0, 2.1 Hz, 1H), 7.60 (s, 1H), 7.36-7.24 (m, 4H), 6.68 (s, 1H), 5.44 (s, 2H), 4.98 (dd, J=13.3 2.15-2.06 (m, 1H).

### Example 7: (2-(2, 6-dioxopiperidin-3-yl)-3-oxo-2, 3-dihydro-1H-imidazo [1, 5-a] indol-7-yl) methyl-(3-chloro-4-((2-(methylamino) ethoxy) methyl) phenyl) carbamate

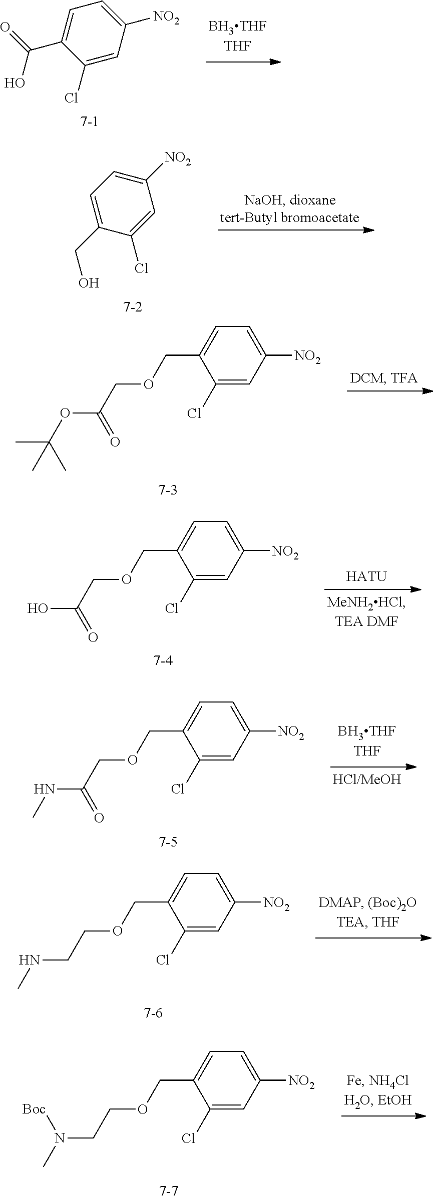

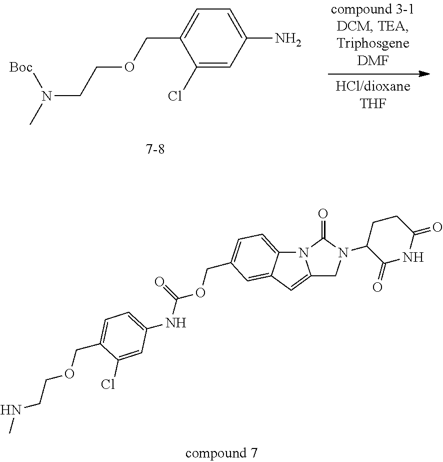 [SMILES: CN(CCOCc1ccc(N)cc1Cl)C(=O)OC(C)(C)C ; CNCCOCc1ccc(NC(=O)OCc2ccc3c(c2)cc2n3C(=O)N(C3CCC(=O)NC3=O)C2)cc1Cl]

### Step 1: 2-chloro-4-nitrobenzyl alcohol (Compound 7-2)

Compound 7-1 (1000 mg, 4.96 mmol) was placed in a reaction tube, the air in the reaction tube was replaced with argon, and tetrahydrofuran (3 mL) was added and stirred. Borane tetrahydrofuran (1 M) (7.5 mL) was slowly added to the reaction tube, which was placed in an oil bath at 60° C. and stirred for 2 hours. After the reaction was completed, methanol (2 mL) was slowly added to quench the reaction, concentrated, and the reaction solution was purified by reversed phase column chromatography (eluent was water:acetonitrile=1:1) to obtain compound 7-2 (675 mg, yield 73%). M/z (ESI): 188 [M+H]⁺.

### Step 2: tert-butyl 2-((2-chloro-4-nitrobenzyl) oxy) acetate (Compound 7-3)

Compound 7-2 (675 mg, 3.6 mmol) and sodium hydroxide (288 mg, 7.2 mmol) were placed in a reaction tube, the air in the reaction tube was replaced with argon, 1, 4-dioxane (4 mL) was added to the reaction tube, stirred, and tert-butyl bromoacetate (2106 mg, 10.8 mmol) was slowly added to the reaction tube, which was placed at room temperature and stirred for 8 hours. After the reaction was completed, the reaction solution was concentrated and purified by reversed phase column chromatography (eluent was water:acetonitrile=1:1) to obtain compound 7-3 (130 mg, yield: 12%). M/z (ESI): 302 [M+H]⁺.

### Step 3: 2-(2-chloro-4-nitrobenzoyloxy) acetic acid (Compound 7-4)

Compound 7-3 (130 mg, 0.43 mmol) was placed in a reaction tube, the air in the reaction tube was displaced with argon, dichloromethane (1 mL) and trifluoroacetic acid (1 mL) were added to the reaction tube, which was stirred at room temperature for 2 hours. After the reaction was completed, the reaction solution was concentrated and purified by reversed phase column chromatography (eluent was water:acetonitrile=1:1) to obtain compound 7-4 (80 mg, 76% yield). M/z (ESI): 246 [M+H]⁺.

### Step 4: 2-((2-chloro-4-nitrobenzyl) oxo)-N-methylacetamide (Compound 7-5)

Compound 7-4 (80 mg, 0.33 mmol), 2-(7-azabenzotriazole)-N, N, N′, N′-tetramethyluronium hexafluorophosphate (190 mg, 0.50 mmol) and methylamine hydrochloride (45 mg, 0.66 mmol) were placed in a reaction tube, the air in the reaction tube was displaced with argon and the reaction tube was stirred in an ice bath. N, N-dimethylformamide (1 mL) and triethylamine (101 mg, 1.0 mmol) were added to the reaction tube, which was stirred at room temperature for 2 hours. After the reaction was completed, the reaction solution was concentrated and purified by reversed phase column chromatography (eluent was water:acetonitrile=1:1) to obtain compound 7-5 (75 mg, yield 89%). M/z (ESI): 259 [M+H]⁺.

### Step 5: 2-((2-chloro-4-nitrophenyl) oxo)-N-methylethane-1-amine (Compound 7-6)

Compound 7-5 (75 mg, 0.29 mmol) was placed in a reaction tube, the air in the reaction tube was displaced with argon, tetrahydrofuran (2 mL) was added and stirred. Dorane tetrahydrofuran (1 M) (0.66 mL) was slowly added to the reaction tube, which was placed in an oil bath at 60° C. and stirred for 2 hours. After the reaction was completed, the reaction was quenched by slowly adding methanol (1 mL), concentrated, and then methanol (1 mL) and hydrochloric acid (10%) (3 mL) were added to the reaction tube, which was stirred in an oil bath at 80° C. for 3 hours. After the reaction was completed, the reaction solution was concentrated and purified by reversed phase column chromatography (eluent was water:acetonitrile=1:1) to obtain compound 7-6 (65 mg, 92% yield). M/z (ESI): 245 [M+H]⁺.

### Step 6: tert-butyl (2-((2-chloro-4-nitrobenzyl) oxo) ethyl) (methyl) carbamate (Compound 7-7)

Compound 7-6 (65 mg, 0.265 mmol), 4-dimethylaminopyridine (2.0 mg, 0.016 mmol) and di-tert-butyl dicarbonate (116.0 mg, 0.53 mmol) were placed in a reaction tube, the air in the reaction tube was displaced with argon, and tetrahydrofuran (3 mL) and triethylamine (101 mg, 1.0 mmol) were added to the reaction tube, which was placed in an oil bath at 50° C., and stirred for 5 hours. After the reaction was completed, the reaction solution was concentrated and purified by reversed phase column chromatography (eluent was water:acetonitrile=1:1) to obtain compound 7-7 (80 mg, 88% yield). M/z (ESI): 345 [M+H]⁺.

### Step 7: tert-butyl (2-((4-amino-2-chlorobenzyl) oxo) ethyl) (methyl) carbamate (Compound 7-8)

Compound 7-7 (80 mg, 0.23 mmol), iron powder (64 mg, 1.15 mmol) and ammonium chloride (37.0 mg, 0.69 mmol) were placed in a reaction tube, the air in the reaction tube was displaced with argon, and ethanol (2 mL) and water (2 mL) were added to the reaction tube, which was placed in an oil bath at 80° C., and stirred for 2 hours. After the reaction was completed, the reaction solution was extracted with ethyl acetate and water, the organic phase was concentrated, and the reaction solution was purified by reverse phase column chromatography (eluent was water:acetonitrile=1:1) to obtain compound 7-8 (55 mg, 75% yield). M/z (ESI): 315 [M+H]⁺.

### Step 8: (2-(2, 6-dioxopiperidin-3-yl)-3-oxo-2, 3-dihydro-1H-imidazo [1, 5-a] indol-7-yl) methyl-(3-chloro-4-((2-(methylamino) ethoxy) methyl) phenyl) carbamate (Compound 7)

Compound 7-8 (20 mg, 0.064 mmol) was placed in a dry reaction tube, the air in the reaction tube was replaced by argon, dichloromethane (2 mL) and triethylamine (13 mg, 0.128 mmol) were added to the reaction tube, which was placed in an ice-water bath at 0° C. and stirred. After cooling down, triphosgene (7.6 mg, 0.025 mmol) was dissolved in dichloromethane (1 mL), and the triphosgene solution was slowly dropped into the reaction solution. Compound 3-1 (10.0 mg, 0.032 mmol) dissolved in N, N-dimethylformamide (1 mL) was added into a reaction tube, and the reaction solution was naturally warmed room temperature and reacted for 2 to 3 hours, and then purified by reversed-phase column chromatography (acetonitrile:water=1:1) to obtain an intermediate. The intermediate was placed in a dry reaction tube, the air in the reaction tube was replaced with argon, tetrahydrofuran (0.5 mL) was added to the reaction tube, stirred in an ice bath, and 1, 4-dioxane hydrogen chloride (4M) (1.5 mL) was added to the reaction tube, stirred in an ice bath for 2 hours. The reaction solution was concentrated at low temperature, and then purified by reversed-phase column chromatography (eluent was water:Acetonitrile=1:1) to obtain compound 7 (3.6 mg, 20% yield) M/z (ESI): 554 [M+H]⁺.

¹H NMR (400 MHz, DMSO-d6) δ 10.00 (s, 1H), 8.32 (s, 1H), 7.87 (d, J=8.3 Hz, 1H), 7.73 (d, J=1.5 Hz, 1H), 7.64 (d, J=1.9 Hz, 1H), 7.47-7.33 (m, 3H), 6.57 (s, 2H), 4.97 (dd, J=13.4 2.10 (dd, J=9.6, 4.2 Hz, 1H).

### Example 8: (2-(2, 6-dioxopiperidin-3-yl)-3-oxo-2, 3-dihydro-1H-imidazo [1,5-a] indol-7-yl) methyl-(3-chloro-4-(methylamino) phenyl) carbamate

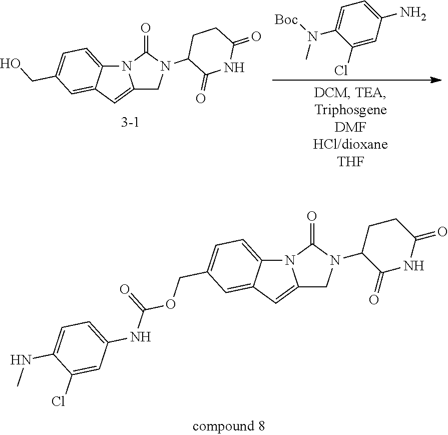 [SMILES: O=C1CCC(N2Cc3cc4cc(CO)ccc4n3C2=O)C(=O)N1 ; CN(C(=O)OC(C)(C)C)c1ccc(N)cc1Cl ; CNc1ccc(NC(=O)OCc2ccc3c(c2)cc2n3C(=O)N(C3CCC(=O)NC3=O)C2)cc1Cl]

Intermediate 2 (16.5 mg, 0.064 mmol) was placed in a dry reaction tube, the air in the reaction tube was replaced by argon, and dichloromethane (2 mL) and triethylamine (13 mg, 0.128 mmol) were added to the reaction tube, which was placed in an ice-water bath at 0° C. and stirred. After cooling down, triphosgene (7.6 mg, 0.025 mmol) was dissolved in dichloromethane (1 mL), the triphosgene solution was slowly dropped into the reaction solution, which was naturally warmed to room temperature and reacted for 0.5 hours. Compound 3-1 (10.0 mg, 0.032 mmol) dissolved in N, N-dimethylformamide (1 mL), was added into a reaction tube and the reaction solution was reacted for 2 to 3 hours at room temperature, and then purified by reversed-phase column chromatography (The eluent is water:acetonitrile=1:1) to obtain an intermediate. The intermediate was placed in a dry reaction tube, the air in the reaction tube was replaced with argon, and tetrahydrofuran (0.5 mL) was added to the reaction tube, stirred in an ice bath. 1, 4-dioxane hydrogen chloride (4M) (1.5 mL) was added to the reaction tube, stirred in an ice bath for 2 hours. The reaction solution was concentrated at low temperature, and then purified by reversed phase column chromatography (eluent was water:Acetonitrile=1:1) to obtain compound 8 (4.5 mg, 28% yield) M/z (ESI): 496 [M+H]⁺.

1H NMR (400 MHz, DMSO-d6) δ 11.06 (s, 1H), 9.48 (s, 1H), 7.86 (d, J=8.3 Hz, 1H), 7.70 (s, 1H), 7.43 (s, 1H), 7.35 (dd, J=8.4, 1.6 Hz, 1H), 7.21 (d, J=8.7 Hz, 1H), 6.62-6.54 (m, 2H), 5.24-5.17 (m, 3H), 4.97 (dd, J=13.3, 5.2 Hz, 1H), 4.61 (dd. J=16.5, 1.7 Hz, 1H)

### Example 9: (2-(2, 6-dioxopiperidin-3-yl)-3-oxo-2, 3-dihydro-1H-imidazo [1,5-a] indol-7-yl) methyl-(4-chloro-3-(2-(2-(methylamino) ethoxy) ethyl) phenyl) carbamate

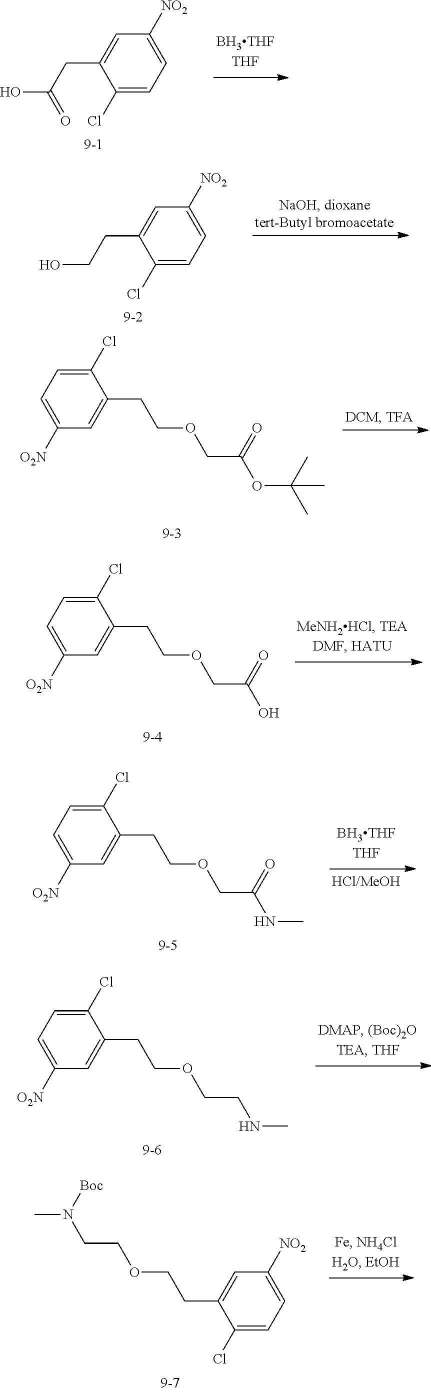

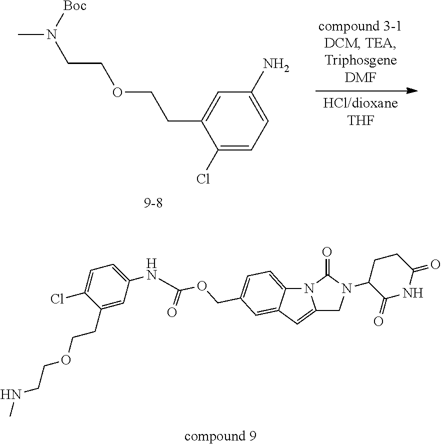 [SMILES: CN(CCOCCc1cc(N)ccc1Cl)C(=O)OC(C)(C)C ; CNCCOCCc1cc(NC(=O)OCc2ccc3c(c2)cc2n3C(=O)N(C3CCC(=O)NC3=O)C2)ccc1Cl]

### Step 1: 2-(2-chloro-5-nitrophenethane) alcohol (Compound 9-2)

Compound 9-1 (1200 mg, 5.55 mmol) was placed in a reaction tube, the air in the reaction tube was displaced with argon, and tetrahydrofuran (5 mL) was added and stirred. Borane tetrahydrofuran (1 M) (8 mL) was slowly added to the reaction tube, which was placed in an oil bath at 60° C. and stirred for 2 hours. After the reaction was completed, menthanol (2 mL) was slowly added to quench the reaction, concentrated, and the reaction solution was purified by reversed phase column chromatography (eluent was water:acetonitrile=1:1) to obtain compound 9-2 (900 mg, 80% yield). M/z (ESI): 202 [M+H]⁺.

### Step 2: tert-butyl 2-(2-chloro-5-nitrophieniethoxy) acetate (Compound 9-3)

Compound 9-2 (900 mg, 4.45 mmol) and sodium hydroxide (356 mg, 8.9 mmol) were placed in a reaction tube, the air in the reaction tube was replaced with argon, and 1, 4-dioxane (5 mL) was added to the reaction tube, stirred, and tert-butyl bromoacetate (2670 mg, 13.6 mmol) was slowly added to the reaction tube, which was placed at room temperature and stirred for 8 hours. After the reaction was completed, the reaction solution was concentrated and purified by reversed phase column chromatography (eluent was water:acetonitrile=1:1) to obtain compound 9-3 (870 mg, 62% yield). M/z (ESI): 316 [M+H]⁺.

### Step 3: 2-(2-chloro-5-nitrophenethoxy) acetic acid (Compound 9-4)

Compound 9-3 (870 mg, 2.75, mmol) was placed into a reaction tube, the air in the reaction tube was displaced with argon, and dichloromethane (1 mL) and trifluoroacetic acid (1 mL) were added to the reaction tube, and the reaction was stirred at room temperature for 2 hours. After the reaction was completed, the reaction solution was concentrated and purified by reversed phase column chromatography (eluent was water:acetonitrile=1:1) to obtain compound 9-4 (640 mg, yield: 89%). M/z (ESI): 260 [M+H]⁺.

### Step 4: 2-(2-chloro-5-nitrophenethoxy)-N-methylacetamide (Compound 9-5)

Compound 9-4 (640 mg, 2.46 mmol), 2-(7-azabenzotriazole)-N, N, N′, N′-tetramethyluronium hexafluorophosphate (1403 mg, 3.69 mmol) and methylamine hydrochloride (332 mg, 4.92 mmol) were placed in a reaction tube, the air in the reaction tube was displaced with argon and the reaction tube was stirred in an ice bath. N, N-dimethylformamide (2 mL) and triethylamine (497 mg, 4.92 mmol) were added to the reaction tube, which was stirred at room temperature for 2 hours. After the reaction was completed, the reaction solution was concentrated and purified by reversed phase column chromatography (eluent was water:acetonitrile=1:1) to obtain compound 9-5 (550 mg, yield: 82%). M/z (ESI): 273 [M+H]⁺.

### Step 5: 2-(2-chloro-5-nitrophenethoxy)-N-methylethan-1-amine (Compound 9-6)

Compound 9-5 (550 mg, 2.0 mmol) was placed in a reaction tube, the air in the reaction tube was displaced with argon, tetrahydrofuran (4 mL) was added and stirred. Borane tetrahydrofuran (1 M) (4 mL) was slowly added to the reaction tube, which was placed in an oil bath at 60° C. and stirred for 2 hours. After the reaction was completed, methanol (1 mL) was slowly added to quench the reaction, concentrated, and methanol (2 mL) and hydrochloric acid (10%) (8 mL) were added to the reaction tube, and the reaction was stirred in an oil bath at 80° C. for 3 hours. After the reaction was completed, the reaction solution was extracted with saturated solution of sodium bicarbonate and ethyl acetate, and the organic phase was concentrated to obtain crude compound 9-6 (508 mg, 98% yield). M/z (ESI): 259 [M+H]⁺.

### Step 6: tert-butyl (2-(2-chloro-5-nitrophenethoxy) ethyl) (methyl) carbamate (Compound 9-7)

Compound 9-6 (508 mg, 1.96 mmol), 4-dimethylaminopyridine (12 mg, 0.098 mmol) and di-tert-butyl dicarbonate (854 mg, 3.92 mmol) were placed in a reaction tube, the air in the reaction tube was displaced with argon, and tetrahydrofuran (10 mL) and triethylamine (594 mg, 5.88 mmol) were added to the reaction tube, which was placed in an oil bath at 50° C., and stirred for 5 hours. After the reaction was completed, the reaction solution was concentrated and purified by reversed phase column chromatography (eluent was water:acetonitrile=1:1) to obtain compound 9-7 (660 mg, yield: 94%). M/z (ESI): 359 [M+H]⁺.

### Step 7: tert-butyl (2-(5-amino-2-chlorophenethoxy) ethyl) (methyl) carbamate (Compound 9-8)

Compound 9-7 (660 mg, 1.84 mmol), iron powder (515 mg, 9.2 mmol) and ammonium chloride (298 mg, 5.5 mmol) were placed in a reaction tube, the air in the reaction tube was displaced with argon, and ethanol (4 mL) and water (4 mL) were added to the reaction tube, which was placed in an oil bath at 80° C., and stirred for 2 hours. After the reaction was completed, the reaction solution was extracted with ethyl acetate and water, the organic phase was concentrated, and the reaction solution was purified by reverse phase column chromatography (eluent was water:acetonitrile=1:1) to obtain compound 9-8 (530 mg, 88% yield). M/z (ESI): 329 [M+H]⁺.

### Step 8: (2-(2, 6-dioxopiperidin-3-yl)-3-oxo-2, 3-dihydro-1H-imidazo [1, 5-a] indol-7-yl) methyl-(4-chloro-3-(2-(2-(methylamino) ethoxy) ethyl) phenyl) carbamate (Compound 9)

Compound 9-8 (21 mg, 0.064 mmol) was placed in a dry reaction tube, the air in the reaction tube was replaced by argon, and dichloromethane (2 mL) and triethylamine (13 mg, 0.128 mmol) were added to the reaction tube, which was placed in an ice-water bath at 0° C. and stirred. After cooling down, triphosgene (7.6 mg, 0.025 mmol) was dissolved in dichloromethane (1 mL), and the triphosgene solution was slowly dropped into the reaction solution, which was naturally warmed to room temperature and reacted for 0.5 hours. Compound 3-1 (10.0 mg, 0.032 mmol) dissolved in N, N-dimethylformamide (1 mL) was added into a reaction tube and the reaction solution was reacted for 2 to 3 hours at room temperature, and then purified by reversed-phase column chromatography (The eluent is water:acetonitrile=1:1) to obtain an intermediate. The intermediate was placed in a dry reaction tube, the air in the reaction tube was replaced with argon, and tetrahydrofuran (0.5 mL) was added to the reaction tube, stirred in an ice bath. 1, 4-dioxane hydrogen chloride (4M) (1.5 mL) was added to the reaction tube, stirred in an ice bath for 2 hours, the reaction solution was concentrated at low temperature, and then purified by reversed phase column chromatography (eluent was water:Acetonitrile=1:1) to obtain compound 9 (7.6 mg, 42% yield) M/z (ESI): 568 [M+H]⁺.

¹H NMR (400 MHz, DMSO-d6) δ 9.89 (s, 1H), 8.33 (s, 1H), 7.87 (d, J=8.3 Hz, 1H), 7.72 (d, J=1.5 Hz, 1H), 7.52 (d, J=2.3 Hz, 1H), 7.40-7.28 (m, 3H), 6.57 (d, J=1.8 Hz, 1H), 5.25 (s, 2H) 2.42-2.32 (m, 4H), 2.14-2.05 (m, 1H).

### Example 10: 1-(3-chloro-4-methylphenyl)-3-((2-(2, 6-dioxopiperidin-3-yl)-3-oxo-2, 3-dihydro-1H-imidazo [1, 5-a] indol-6-yl) methyl) urea

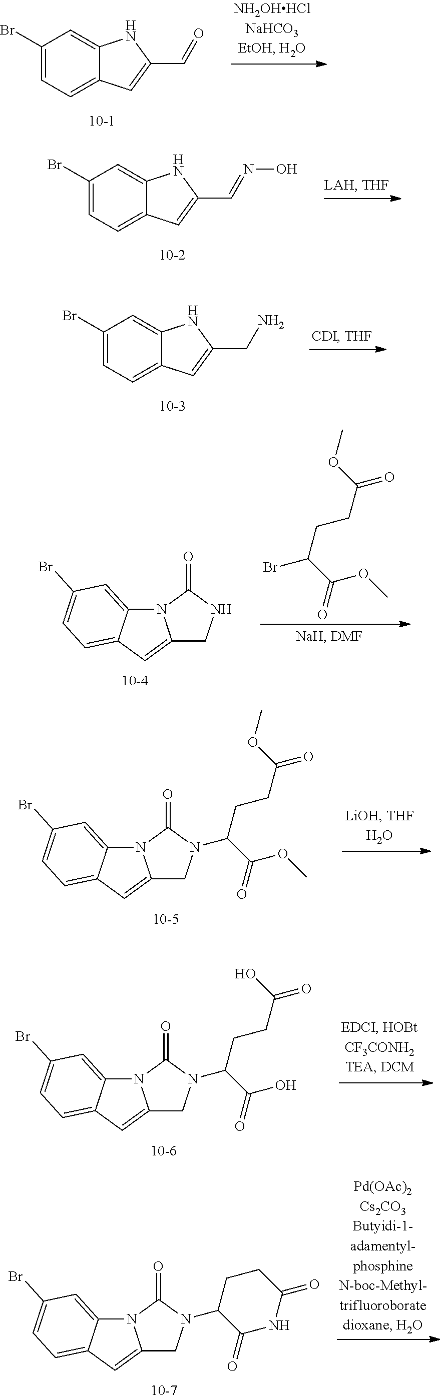

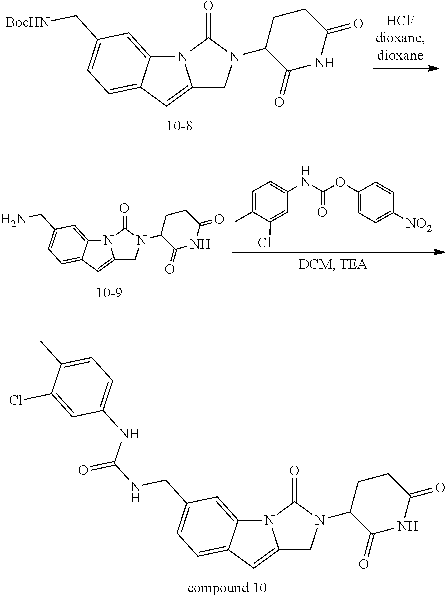

### Step 1: 5-bromo-1H-indole-2-carboxaldehyde oxime (Compound 10-2)

Compound 10-1 (1000 mg, 4.46 mmol) hydroxylamine hydrocholoride (465 mg, 6.69 mmol), sodium bicarbonate (749 mg, 8.92 mmol) were placed in a flask, ethanol (15 mL) and water (10 mL) were added and the reaction solution was stirred in an oil bath at 70° C. for 3 h. After the reaction was completed, the reaction solution was concentrated, extracted with ethyl acetate and water, and the organic phase was dried and concentrated to obtain the crude product compound 10-2 (1010 mg, 95% yield). M/z (ESI): 239 [M+H]⁺.

### Step 2: (6-bromo-1H-indol-2-yl) methylamine (Compound 10-3)

Compound 10-2 (1010 mg, 4.22 mmol) was placed in a flask, anhydrous tetrahydrofuran (25 mL) was added, and the reaction solution was placed in an ice bath at 0° C. and stirred to cool down. After lowering the temperature, lithium aluminum tetrahydride (240 mg, 6.33 mmol) was slowly added, then the reaction solution was placed in an oil bath at 70° C. and stirred for 2 h. After the reaction was completed, the reaction solution was stirred in an ice bath at 0° C. to cool down, sodium sulfate decahydrate was slowly added to quench the reaction. After quenching, the reaction solution was filtered, and the filtrate was extracted with ethyl acetate and water, and the organic phase reaction solution was concentrated to obtain a crude product, which was purified by reverse phase column chromatography (eluent was water:acetonitrile=1:1) to obtain compound 10-3 (694 mg, yield 73%). M/z (ESI): 225 [M+H]⁺.

### Step 3: 6-bromo-1, 2-dihydro-3H-imidazo [1,5-a] indol-3-one (Compound 10-4)

Compound 10-3 (694 mg, 3.08 mmol) was placed into a dry sealed tube, the air in the reaction tube was displaced with argon, tetrahydrofuran (3 mL) was added and the dry sealed tube was sealed. The reaction solution was placed in an ethanol-dry ice bath, stirred and cooled down. N, N′-carbonyldiimidazole (550 mg, 3.39 mmol) was placed in a dry flask, and tetrahydrofuran (3 mL) was added to dissolve it. N, N′-carbonyldiimidazole solution was slowly added dropwise to the reaction solution. After the reaction was completed, the reaction solution was concentrated to obtain a crude product, which was purified by normal phase column chromatography (eluent was petroleum ether:ethyl acetate=1:1) to obtain compound 10-4 (526 mg, yield 68%). M/z (EST): 251 [M+H]⁺.

### Step 4: dimethyl 2-(6-bromo-3-carbonyl-1H-imidazo [1,5-a] indol-2 (3H)-yl) glutarate (Compound 10-5)

Compound 10-4 (526 mg, 2.10 mmol) and sodium hydride (75.2 mg, 3.14 mmol) were placed in a dry reaction tube, the air in the reaction tube was replaced with argon, the reaction tube was placed in an ethanol-dry ice bath to cool down, and N, N-dimethylformamide (2 mL) was added to the reaction tube, the reaction solution was naturally warmed to room temperature and stirred for half an hour, and then stirred in an ice-water bath. Dimethyl 2-bromoglutarate (749 mg, 3.14 mmol) was added dropwise to the reaction solution, which was continued stirring in an ice-water bath for half an hour and then stirred at room temperature for one hour. After the reaction was completed, acetic acid (250 mg, 4.2 mmol) was added to quench the reaction, and the reaction solution was purified by reverse phase column chromatography (eluent was water:acetonitrile=1:1) to obtain compound 10-5 (686 mg, yield 80%). M/z (ESI): 409 [M+H]⁺.

### Step 5: 2-(6-bromo-3-oxo-1H-imidazo [1,5-a] indol-2 (3H)-yl) glutaric acid (Compound 10-6)

Compound 10-5 (686 mg, 1.68 mmol) and lithium hydroxide (161 mg, 6.72 mmol) were placed in a flask, tetrahydrofuran (4 mL) and water (2 mL) were added, and stirred at room temperature for 3-5 hours. After the reaction was completed, hydrochloric acid (3N) was added to adjust the pH value of the reaction solution to 3-5, the reaction solution was concentrated, and then purified by reversed-phase column chromatography (eluent was water:acetonitrile=1:1) to obtain compound 10-6 (170 mg, yield: 90%). M/z (ESI): 381 [M+H]⁺.

### Step 6: 3-(6-bromo-3-oxo-1H-imidazo [1, 5-a] indol-2 (3H)-yl) piperidine-2,6-dione (Compound 10-7)

Compound 10-6 (575 mg, 1.51 mmol), trifluoroacetamide (255 mg, 2.26 mmol), 1-hydroxybenzotriazole (448 mg, 3.32 mmol) and 1-ethyl-(3-dimethylaminopropyl) carbondiimide hydrochloride (1012 mg, 5.28 mmol) were placed into a dry reaction tube, the air in the reaction tube was displaced with argon, the reaction tube was placed in an ethanol-dry ice bath to cool down. Dichloromethane (10 mL) and triethylamine (687 mg, 6.80 mmol) was added in reaction tube, and the reaction solution was naturally warmed to room temperature and stirred for half an hour, then placed in an oil bath at 35° C. and stirred for 5 hours. After the reaction was completed, the reaction solution was concentrated and purified by reverse phase column chromatography (eluent was water:acetonitrile=1:1) to obtain compound 10-7 (448 mg, yield: 82%). M/z (EST): 362 [M+H]⁺.

### Step 7: tert-butyl [(2-(2, 6-dioxo-3-oxo-1H-imidazo [1,5-a] indol-6-yl) methyl) carbamate (Compound 10-8)

Compound 10-7 (80 mg, 0.22 mmol), palladium acetate (5.0 mg, 0.022 mmol), n-butylbis (1-adamantyl) phosphine (15.8 mg, 0.044 mmol), (((tert-butoxycarbonyl) amino) methyl) trifluoroborate potassium salt (68.0 mg, 0.028 mmol) and cesium carbonate (215.0 mg, 0.66 mmol) were placed in a reaction tube, and the air in the reaction tube was replaced with argon. After the reaction was completed, the reaction solution was purified by reverse phase column chromatography (eluent was water:acetonitrile=1:1) to obtain compound 10-8 (296 mg, 58% yield). M/z (ESI): 413 [M+H]+.

### Step 8: 3-(6-aminomethyl)-3-oxo-1H-imidazo [1, 5-a] indol-2 (3H)-yl) piperidine-2, 6-dione (Compound 10-9)

Compound 10-8 (11.0 mg, 0.027 mmol) was placed in a reaction tube, the air in the reaction tube was displaced with argon, 1, 4-dioxane (2 mL) and 1, 4-dioxane-hydrogen chloride (4M)(2 mL) were added to the reaction tube, which was placed in an oil bath at 25° C. and stirred for 5 hours. After the reaction was completed, the reaction solution solvent was removed by spin-dry to obtain crude compound 10-9 (9.2 mg, yield: 99%). M/z (ESI): 313 [M+H]⁺.

### Step 9: 1-(3-chloro-4-methylphenyl)-3-((2-(2, 6-dioxopiperidin-3-yl)-3-oxo-2, 3-dihydro-1H-imidazo [1, 5-a] indol-6-yl) methyl) urea (Compound 10)

Compound 10-9 (9.2 mg, 0.027 mmol) and (4-nitrophenyl)-N-(3-chloro-4-methyl-phenyl) carbamate (Intermediate 1, 9.8 mg, 0.032 mmol) were placed in a dry reaction tube, the air in the reaction tube was displaced with argon, dichloromethane (2 mL) and triethylamine (5.94 mg, 0.057 mmol) were added to the reaction tube, which was placed in an oil bath at 25° C. and stirred for 4 hours. After the reaction was completed, the reaction solution was concentrated to obtain a crude product, which was purified by reverse phase column chromatography (eluent was water:acetonitrile=1:1) to obtain compound 10 (5.1 mg, 39% yield). M/z (ESI): 480 [M+H]⁺.

¹H NMR (400 MHz, DMSO-d6) δ 11.04 (s, 1H), 8.75 (s, 1H), 7.81 (s, 1H), 7.66 (d, J=2.1 Hz, 1H), 7.59 (d, J=8.2 Hz, 1H), 7.22-7.11 (m, 3H), 6.81 (t, J=5.9 Hz, 1H), 6.50 (s, 1H), 4.96 (dd, J=13.4, 5.1 Hz, 1H), 4.59 (d, J=16.5 Hz, 1H),

### Example 11: 1-(3-chloro-4-methylphenyl)-3-((2-(2, 6-dioxopiperidin-3-yl)-3-oxo-2, 3-dihydro-1H-imidazo [1, 5-a] indol-9-yl) methyl) urea

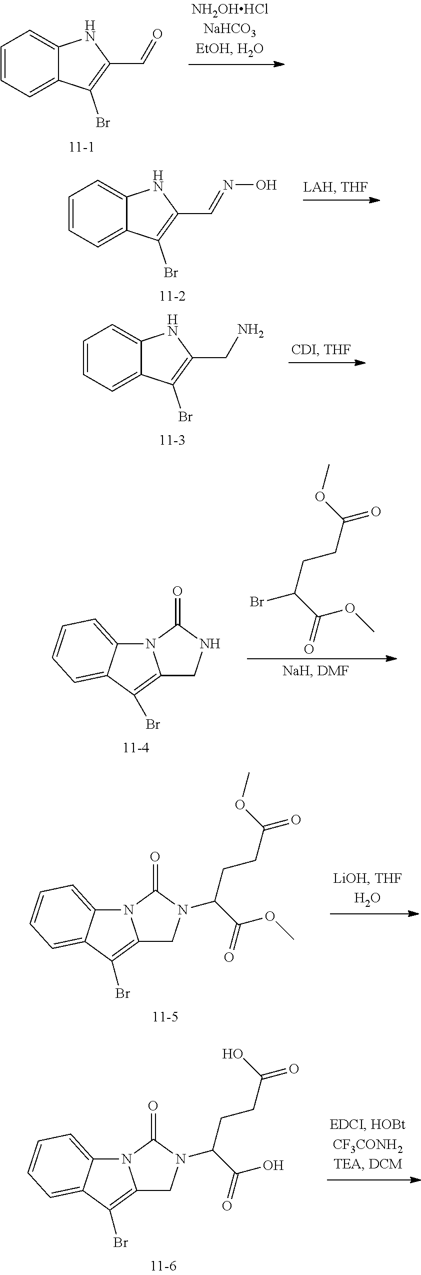

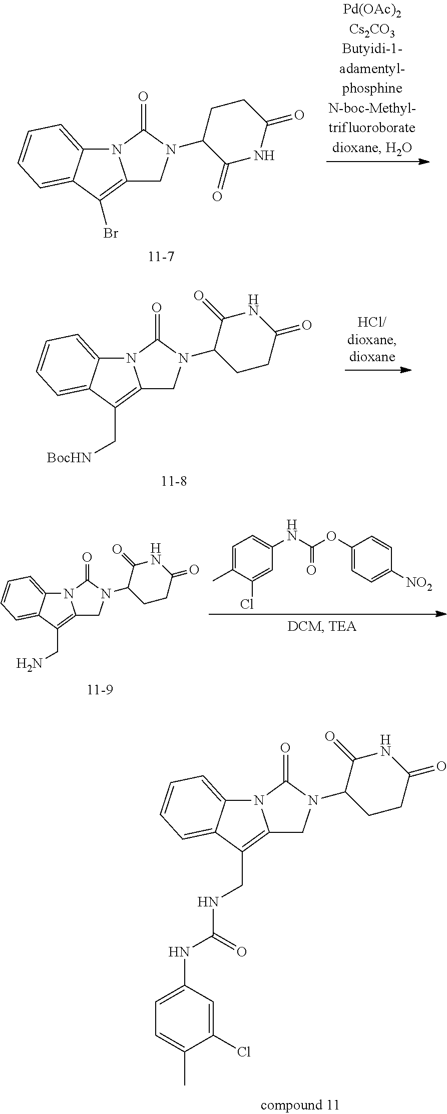

### Step 1: 3-bromo-1H-indole-2-carboxaldehyde oxime (Compound 11-2)

Compound 11-1 (1000 mg, 4.46 mmol) hydroxylamine hydrochloride (465 mg, 6.69 mmol), sodium bicarbonate (749 mg, 8.92 mmol) were placed in a flask, ethanol (15 mL) and water (10 mL) were added, and the reaction solution was stirred in an oil bath at 70° C. for 3 h. After the reaction was completed, the reaction solution was concentrated, extracted with ethyl acetate and water, and the organic phase was dried and concentrated to obtain crude compound 11-2 (1010 mg, 95% yield).

M/z (ESI): 239 [M+H]⁺.

### Step 2: (3-bromo-1H-indol-2-yl) methylamine (Compound 11-3)

Compound 11-2 (1010 mg, 4.22 mmol) was placed in a flask, anhydrous tetrahydrofuran (25 mL) was added, and the reaction solution was placed in an ice bath at 0° C. and stirred to cool down. After lowering the temperature, lithium aluminum tetrahydride (240 mg, 6.33 mmol) was slowly add, then the reaction solution was placed in an oil bath at 70° C. and stirred for 2 h. After the reaction was completed, the reaction solution was stirred in an ice bath at 0° C. to cool down, sodium sulfate decahydrate was slowly added to quench the reaction. After quenching, the reaction solution was filtered, and the filtrate was extracted with ethyl acetate and water, and the organic phase reaction solution was concentrated to obtain a crude product, which was purified by reversed phase column chromatography (eluent was water:acetonitrile=1:1) to obtain compound 11-3 (694 mg, yield 73%). M/z (ESI): 225 [M+H]⁺.

### Step 3: 9-bromo-1, 2-dihydro-3H-imidazo [1,5-a] indol-3-one (Compound 11-4)

Compound 11-3 (694 mg, 3.08 mmol) was placed in a dry sealed tube, the air in the reaction tube was replaced with argon, tetrahydrofuran (3 mL) was added and the reaction tube was sealed. The reaction solution was placed in an ethanol-dry ice bath, stirred and cooled down. N, N′-carbonyldiimidazole (550 mg, 3.39 mmol) was placed in a dry flask, tetrahydrofuran (3 mL) was added to dissolve it. N, N′-carbonyldiimidazole solution was slowly added dropwise to the reaction solution. After the reaction was completed, the reaction solution was concentrated to obtain a crude product, which was purified by normal phase column chromatography (eluent was petroleum ether:ethyl acetate=1:1) to obtain compound 11-4 (526 mg, yield 68%). M/z (ESI): 251 [M+H]⁺.

### Step 4: Dimethyl 2-(9-bromo-3-oxo-1H-imidazo [1,5-a] indol-2 (3H)-yl) glutarate (Compound 11-5)

Compound 11-4 (526 mg, 2.10 mmol) and sodium hydride (75.2 mg, 3.14 mmol) were placed in a dry reaction tube, the air in the reaction tube was replaced with argon, the reaction tube was placed in an ethanol-dry ice bath to cool down. N, N-dimethylformamide (2 mL) was added to the reaction tube, and the reaction solution was naturally warmed to room temperature and stirred for half an hour, and then stirred in an ice-water bath. Dimethyl 2-bromoglutarate (749 mg, 3.14 mmol) was added dropwise to the reaction solution, which was continued stirring in an ice-water bath for half an hour and then stirred at room temperature for one hour. After the reaction was completed, acetic acid (250 mg, 4.2 mmol) was added to quench the reaction, and then the reaction solution was purified by reverse phase column chromatography (eluent was water:acetonitrile=1:1) to obtain compound 11-5 (686 mg, yield 80%). M/z (ESI): 409 [M+H]⁺.

### Step 5: 2-(9-bromo-3-oxo-1H-imidazo [1, 5-a] indol-2 (3H)-yl) glutaric acid (Compound 11-6)

Compound 11-5 (686 mg, 1.68 mmol) and lithium hydroxide (161 mg, 6.72 mmol) were placed in a flask, tetrahydrofuran (4 mL) and water (2 mL) were added, and stirred at room temperature for 5 hours. After the reaction was completed, hydrochloric acid (3N) was added to adjust the pH value of the reaction solution to 3-5, the reaction solution was concentrated, and then purified by reversed-phase column chromatography (eluent was water:acetonitrile=1:1) to obtain compound 11-6 (170 mg, yield: 90%). M/z (ESI): 381 [M+H]⁺.

### Step 6: 3-(9-bromo-3-oxo-1H-imidazo [1, 5-a] indol-2 (3H)-yl) piperidine-2,6-dione (Compound 11-7)

Compound 11-6 (90 mg, 0.236 mmol), trifluoroacetamide (40 mg, 0.354 mmol), 1-hydroxybenzotriazole (70 mg, 0.519 mmol) and 1-Ethyl-(3-dimethylaminopropyl) carbondiimide hydrochloride (158.0 mg, 0.826 mmol) were placed in a dry reaction tube, the air in the reaction tube was displaced with argon, the reaction tube was placed in an ethanol-dry ice bath to cool down. Dichloromethane (10 mL) and triethylamine (107.0 mg, 1.0 mmol) were added to the reaction tube, and the reaction solution was naturally warmed to room temperature and stirred for half an hour, and then stirred in an oil bath at 35° C. for 5 hours. After the reaction was completed, the reaction solution was concentrated and purified by reversed phase column chromatography (eluent was water:acetonitrile=1:1) to obtain compound 11-7 (20.0 mg, yield: 23%). M/z (ESI): 362 [M+H]⁺.

### Step 7: tert-butyl 1(2-(2, 6-dioxopiperidin-3-yl)-3-oxo-2, 3-dihydro-1H-imidazo [1, 5-a] indol-9-yl) methyl) carbamate (Compound 11-8)

Compound 11-7 (20 mg, 0.055 mmol), palladium acetate (2.0 mg, 0.008 mmol), n-butylbis (1-adamantyl) phosphine (6.0 mg, 0.016 mmol), (((tert-butoxycarbonyl) amino) methyl) trifluoroborate potassium salt (13.0 mg, 0.055 mmol) and cesium carbonate (54.0 mg, 0.166 mmol) were placed in a reaction tube, the air in the reaction tube was displaced with argon, 1, 4-dioxane (3 mL) and water (0.3 mL) were added to the reaction tube. After the reaction was completed, the reaction solution was purified by reverse phase column chromatography (eluent was water:acetonitrile=1:1) to obtain compound 11-8 (16.0 mg, yield: 70%). M/z (ESI): 413 [M+H]⁺.

### Step 8: 3-(9-aminomethyl)-3-oxo-1H-imidazo [1,5-a] indol-2 (3H)-yl) piperidine-2, 6-dione (Compound 11-9)

Compound 11-8 (16.0 mg, 0.038 mmol) was placed in a reaction tube, the air in the reaction tube was displaced with argon, 1, 4-dioxane (2 mL) and 1, 4-dioxane-hydrogen chloride (4M)(2 mL) were added to the reaction tube, which was placed in an oil bath at 25° C. and stirred for 5 hours. After the reaction was completed, the reaction solution solvent was removed by spin-dry to obtain crude compound 11-9 (11.0 mg, yield: 90%). M/z (ESI): 313 [M+H]⁺.

### Step 9: 1-(3-chloro-4-methylphenyl)-3-((2-(2, 6-dioxopiperidin-3-yl)-3-oxo-2, 3-dihydro-1H-imidazo [1, 5-a] indol-9-yl) methyl) urea (Compound 11)

Compound 11-9 (11 mg, 0.035 mmol) and (4-nitrophenyl) N-(3-chloro-4-methyl-phenyl) carbamate (Intermediate 1, 13.0 mg, 0.042 mmol) were placed in a dry reaction tube, the air in the reaction tube was displaced with argon, and dichloromethane (2 mL) and triethylamine (7.8 mg, 0.077 mmol) were added to the reaction tube, which was placed in an oil bath at 25° C. and stirred for 4 hours. After the reaction was completed, the reaction solution was concentrated to obtain a crude product, which was purified by reverse phase column chromatography (eluent was water:acetonitrile=1:1) to obtain compound 11 (7.5 mg, yield 44%). M/z (ESI): 480 [M+H]⁺.

¹H NMR (400 MHz, DMSO-d6) δ 11.05 (s, 1H), 8.85 (s, 11H), 7.86-7.79 (m, 1H), 7.73 (d. J=6.7 Hz, 1H), 7.63 (d, J=2.0 Hz, 1H), 7.34-7.22 (m, 2H), 7.20-7.08 (m, 2H), 6.83 (t, J=5.8 Hz, 1H), 4.95 (dd, J=13.3, 5.1 Hz, 1H)

### Example 12: N-(3-chloro-4-methylphenyl)-3-(2-(2, 6-dioxopiperidin-3-yl)-3-oxo-2, 3-dihydro-1H-imidazo [1,5-a] indol-7-yl) propanamide

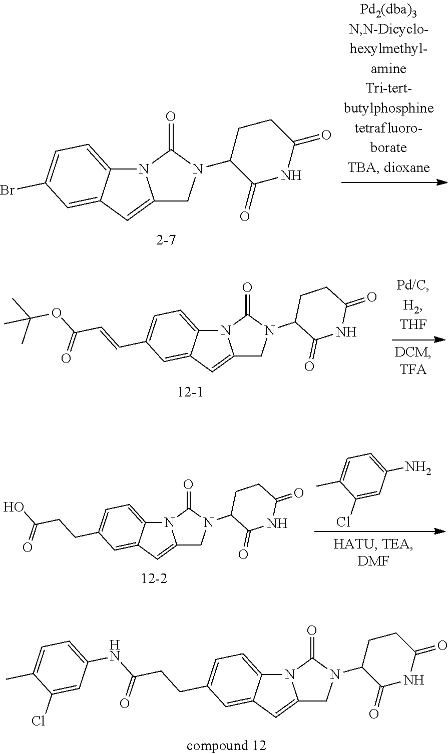

### Step 1: tert-butyl (E)-3-(2-(2, 6-dioxopiperidin-3-yl)-3-oxo-2, 3-dihydro-1H-imidazo [1,5-a]indol-7-yl) acrylate (Compound 12-1)

Compound 2-7 (35 mg, 96.64 μmol), tris (dibenzylideneacetone) dipalladium (17.68 mg, 19.33 μmol), tri-tert-butylphosphine tetrafluoroborate (15.37 mg, 53.15 μmol) and N, N-dicyclohexylmethylamine (33.38 mg, 0.17 mmol) were placed in a dry reaction tube and the air in the reaction tube was replaced with nitrogen. 1, 4-Dioxane (1 mL) and tert-butyl acrylate (32.82 mg, 256.09 μmol) were added with a syringe. The reaction solution was warmed to 60° C. and stirred for 16 hours, then detected by LC-MS. After the reaction was completed, the reaction solution was concentrated and purified by reversed phase column chromatography (eluent was water:acetonitrile=1:1) to obtain compound 12-1 (26 mg, yield 66%). M/z (ESI): 410 [M+H]⁺.

### Step 2: 3-(2-(2, 6-dioxopiperidin-3-yl)-3-oxo-2, 3-dihydro-1H-imidazo [1,5-a] indol-7-yl) propanoic acid (Compound 12-2)

Compound 12-1 (26 mg, 0.063 mmol) and palladium on carbon (7.0 mg 55% water content) and THF (2 mL) were placed in a dry reaction tube, and the air in the reaction tube was replaced with hydrogen. The reaction solution was stirred at 25° C. for 6 hours, then detected by LC-MS. After the reaction was completed, the reaction solution is filtered through a filter to obtain a clear solution, and the solvent was removed by a rotary evaporator to obtain an intermediate product. Then dichloromethane (1.0 mL) and trifluoroacetic acid (0.5 mL) were added to the reaction tube, which was stirred at room temperature for 6 hours. The reaction was then detected by LC-MS. After the reaction was completed, the solvent was removed by a rotary evaporator to obtain a crude product, which was purified by reverse phase column chromatography (eluent was water:acetonitrile=1:1) to obtain compound 12-2 (14 mg, 62% yield). M/z (ESI): 356 [M+H]⁺.

### Step 3: N-(3-chloro-4-methylphenyl)-3-(2-(2, 6-dioxopiperidin-3-yl)-3-oxo-2, 3-dihydro-1H-imidazo [1,5-a] indol-7-yl) propanamide (Compound 12)

Compound 12-2 (14 mg, 0.039 mmol), 3-chloro-p-toluidine (8.4 mg, 0.059 mmol), 2-(7-azobenzotriazole)-N, N, N′, N′-tetramethyluronium hexafluorophosphate (22.30 mg, 0.059 mmol) were placed in a dry reaction tube, the air in the reaction tube was replaced with argon, and N, N-dimethylformamide (2 mL) and triethylamine (7.97 mg, 0.079 mmol) were added into the reaction tube, and the reaction was carried out at room temperature for 2 to 3 hours. The reaction solution was purified by reverse phase column chromatography (eluent water:acetonitrile=1:1) to obtain compound 12 (11.2 mg, 59% yield). M/z (ESI): 479 [M+H]⁺.

¹H NMR (400 MHz, DMSO-d6) δ 11.04 (s, 1H), 10.00 (s, 1H), 7.79 (d. J=2.1 Hz, 1H), 7.75 (d. J=8.3 Hz, 1H), 7.49 (s, 1H), 7.32 (dd. J=8.3, 2.2 Hz, 1H), 7.24 (d. J=8.4, 1.6 Hz, 1H), 7.18 (dd, J=8.3, 1.6 Hz, 1H) 3H), 2.14-2.04 (m, 1H).

### Example 13: 2-(2, 6-dioxopiperidin-3-yl)-3-oxo-2, 3-dihydro-1H-imidazo [1,5-a] indol-8-yl) methyl (3-chloro-4-methylphenyl) carbamate

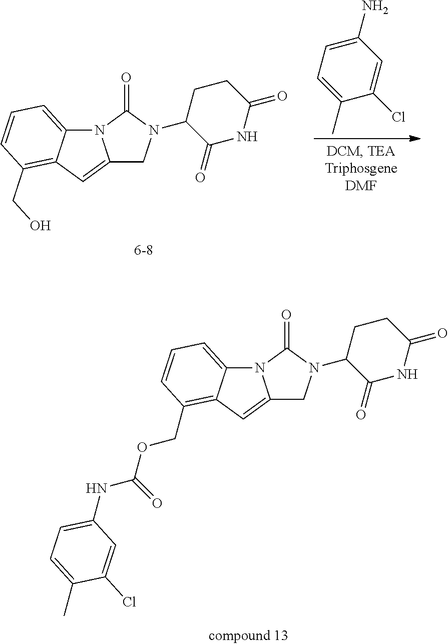

3-chloro-p-toluidine (10.8 mg, 0.076 mmol) was placed in a dry reaction tube, the air in the reaction tube was replaced by argon, and dichloromethane (2 mL) and triethylamine (15 mg, 0.153 mmol) were added to the reaction tube, which was placed in an ice-water bath at 0° C. and stirred. After cooling down, triphosgene (9 mg, 0.03 mmol) was dissolved in dichloromethane (1 mL), and the triphosgene solution was slowly dropped into the reaction solution, which was naturally warmed to room temperature for 0.5 hours, then the solvent was removed by spin-dry. Compound 6-8 (18.0 mg, 0.057 mmol) dissolved in N, N-dimethylformamide (1 mL) was added into a reaction tube, and the reaction was carried out at room temperature for 2-3 hours, and then the reaction solution was purified by reversed phase column chromatography (the eluent was water:acetonitrile=1:1) to obtain compound 13 (11.2 mg, yield 30%). M/z (ESI): 481 [M+H]⁺.

¹H NMR (400 MHz, DMSO-d6) δ 11.06 (s, 1H), 9.86 (s, 1H), 7.86 (dd, J=7.2, 2.0 Hz, 1H), 7.60 (d, J=2.1 Hz, 1H), 7.36-7.20 (m, 4H), 6.69 (s, 1H), 5.43 (s, 2H), 4.98 (dd, J=13.4, 5.1 Hz, 1H), 4.63 (dd, J=16.7, 1.7 Hz, 1H)

### Example 14: 3-(1-oxo-1H-benzo [d] imidazo [1,5-a] imidazol-2 (3H)-yl) piperidine-2, 6-dione

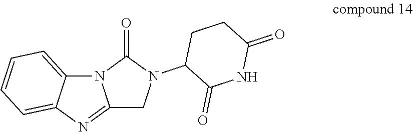 [SMILES: O=C1CCC(N2Cc3nc4ccccc4n3C2=O)C(=O)N1]

Compound 14 (9 mg, 6% yield) was prepared by replacing the starting material compound 1-1 with (1H-benzimidazol-2-methylene) amine with reference to the synthetic procedure of Example 1. M/z (ESI): 285 [M+H]⁺.

¹H NMR (400 MHz, DMSO-d6) δ 11.08 (s, 1H), 8.43 (s, 1H), 7.86-7.73 (m, 2H), 7.40-7.37 (m, 1H), 5.04 (dd, J=13.2, 5.0 Hz, 1H), 4.74-4.53 (m, 2H), 2.95-2.88 (m, 2H), 2.40-2.32 (m, 2H).

Compounds other than those synthesized in Examples 1-14 can be synthesized by referring to the synthetic pathways and source materials in Examples 1-14.

### Test Example for Biological Activity and Related Properties

### Test Example 1, Cereblon Binding Experiment

### 1. Experimental Instruments and Materials

| | Equipment | |
|---|---|---|
| Instrument Name | manufacturer | Model |
| Oscillator | Boxun | BSD-YX3400 |
| Plate reader | PerkinElmer | Envision |
| Centrifuge | Eppendorf | Microplate Shaker, Catalog # |
| | | 5353 |
| Compound dilution and | PerkinElmer | Echo |
| loading apparatus | | |

The detection kit (HTRF Human Cereblon Binding Kits) used in the experiment was a detection method for quantitative measurement of Cereblon WT ligand using HTRF® technology. The detection principle was based on HTRF technology. The specifically labeled GST antibody (Eurum Cryptate, donor) bound to the GST-labeled human Cereblon WT ligand and the XL665-labeled lenalidomide tracer (acceptor) at the same time. The donor was excited with a light source to initiate fluorescence resonance energy transfer (FRET) to the acceptor. The acceptor emitted fluorescence at a specific wavelength of 665 nm. After adding the compound, the compound competed with XL665-labeled lenalidomide to prevent FRET. The FRET signal ratio was inversely proportional to the compound concentration.

The information of other reagents and consumables required for the experiment was as follows:

| Reagent | Brand | Item number |
|---|---|---|
| HTRF Human Cereblon Binding Kits | Promega | 64BDCRBNPEG |
| 384-well plate | PerkinElmer | 6007299 |
| Lenalidomide | Shaoyuan | SY047646 |

### 2. Experimental Steps

Compounds of the present disclosure were dissolved in DMSO, with a mother liquor storage concentration of 10 mM. The gradient dilution of the compound mother liquor was carried out through the dose-response program of the compound dilution and loading instrument. The total experimental volume of the dilution program was 20 μL, with the initial concentration of 100 μM for the tested compound and 200 μM for the standard sample. A 4-fold dilution was performed, resulting in 8 concentration points, and the DMSO content was 1×%. After the procedure, 5 μL of 19 # dilution from the kit was added to each well, and then 5 μL of GST-labeled human Cereblon WT ligand was added. After being mixed well, 10 μL of HTRF detection reagent was added, and incubated at room temperature for 3 hours. The HTRF signal in each well was measured using an Envision plate reader. 100% binding inhibition is defined as the signal ratio under treatment with 200 μM standard lenalidomide.

### 3. Data Analysis

The ratio of acceptor and donor emission signals was calculated for each well:

$$Ratio = 665 nm \text{signal/620} nm signal$$

$$Coefficient of deviation ( \% ) = \text{Standard deviation/mean ratio}$$

$$Inhibition rate of Cereblon binding ( \% ) = 100 \% - 100 \% \times ( Sample - L \text{)/(} H - L )$$

Wherein:
Sample=Ave (test sample group);
H=Ave (DMSO treatment group);
L=Ave (200 μM lenalidomide standard treatment group).

Data analysis was performed by GraphPad Prism 9. The concentration-effect curves were obtained using a nonlinear four-parameter fitting, and IC₅₀ of compounds was calculated:

$$Y = Bottom + ( Top - Bottom \text{)/(} 1 + 10 \hat{} ( ( Log {IC}_{50} - X ) * HillSlope ) )$$

Wherein:
X: Log compound concentration;
Y: inhibition rate (%);
Bottom is the minimum percent inhibition:
Top is the maximum percent inhibition:
HillSlope is the slope coefficient of the curve.

The binding ability of the compounds of the present disclosure to Cereblon was determined by the above assay, and the measured IC₅₀ values were shown in Table 1.

TABLE 1

| Compound Number | IC₅₀ (μM) |
|---|---|
| Compound 1 | B |
| Compound 14 | B |

A: IC₅₀ < 1 μM; B: 1 μM ≤ IC₅₀ < 10 μM; C: 10 μM ≤ IC₅₀ < 100 μM; D: IC₅₀ ≥ 100 μM.

### Test Example 2: Anti-Proliferative Activity Experiment on CAL51 Cells

1 [symbol] Laboratory Instruments and Materials

### Instruments and Equipment

| | | Equipment |
|---|---|---|
| Instrument Name | Model | manufacturer |
| Centrifuge | 5800 | Eppendorf |
| Cytometer | IC1000 | Count star |
| 4° C. refrigerator | YG-330L | Thermo |
| Inverted microscope | TS2 | Nikon |
| Cell incubator | 1IVIOS 250i | Thermo |
| Envision plate reader | Envision2105 | Perkin Elmer |

### Experimental Reagents and Consumables

| Name | Item number | Brand |
|---|---|---|
| DMEM medium | 11995-065 | Gibco |
| FBS | 10099-141C | Gibco |
| Trypsin-EDTA (0.25%), phenol red | 25200056 | Gibco |
| PBS | SH30256.01 | Cytiva |
| CellTiter-Glo ® 3D Cell Viability | G9683 | Promega |
| Assay | | |
| 96-well black/transparent round | 4515 | Corning |
| bottom ultra-low adsorption plate | | |

2 [symbol] Experimental Procedure

### (1) Cell Plating

The medium of target cells CAL51 (CBP60360, Kebai) was removed, and PBS was added for rinsing once, and then digested with trypsin (Trypsin-EDTA (0.25%)) for 5 minutes. After digestion, 10 mL of complete medium (DMEM containing 10% FBS) was added to neutralize trypsin, and the cells were pipetted and collected, then centrifuged at 300 g for 5 min, counted. The cell density was adjusted to 40,000 cells/mL. 90 μL cell suspension was added to a 96-well low adsorption plate. 200 μL of PBS was added to the edge wells. The plate was centrifuged at 300 g for 5 minutes to form cell spheroid, which were then incubated in a cell incubator overnights.

(2) Add drug to the cells The compounds of the present disclosure were dissolved in DMSO, with a mother liquor storage concentration of 10 mM. Before drug administration, the compound was subjected to a 10-fold gradient dilution using DMSO, resulting in 6 gradient working solutions. 2 μL of each working solution of different concentration was added to the dilution plate containing 198 μL culture medium, and the mixtures were mixed thoroughly by pipetting. 10 μL of medium containing compound from the dilution plate was add to a cell plate containing 90 μL of cell suspension plated the day before. The final concentrations of each gradient compound were 10,000 nM, 1000 nM, 100 nM, 10 nM, 1 nM, 0.1 nM. The positive control compound was CC-885. The diluted compound was added at 10 μL per well, then the plate was centrifuged and incubated in a carbon dioxide incubator for 3 days.

### (3) CellTiter-Glo® 3D Cell Viability Assay Test

The cells were removed from the incubator and returned to room temperature for 30 mm. 50 μL of CellTiter-Glo® 3D Cell Viability Assay reagent was added, and the plate was shaked and mixed well for 10 minutes, and then the plate was read with an Envision plate reader.

### 3. Data Analysis

The anti-proliferative activity of the compound of the present disclosure on CAL51 cells was measured by the above assay, and the cell growth inhibition rate of each sample well was calculated based on the raw data.

$$Inhibition rate ( \% ) = 100 \% * ( 1-sample \text{reading/DMSO} reference mean reading )$$

Sample reading: refers to the signal value of the experimental group;
DMSO reference mean reading: refers to the mean signal value of the DMSO control group. The DMSO control group did not add test compounds, and other operations were consistent with those of the experimental group.

Data analysis was performed by GraphPad Prism 9. The concentration-effect curves were obtained using a non-linear four-parameter fitting, and IC₅₀ of compounds was calculated.

$$Y = Bottom + ( Top - Bottom \text{)/(} 1 + 10 \hat{} ( ( Log {IC}_{50} - X ) * HillSlope ) )$$

Wherein:
X: Log of the compound concentration;
Y: inhibition rate (%);
Bottom is the minimum percent inhibition;
Top is the maximum percentage inhibition;
HillSlope is the slope coefficient of the curve.

The anti-proliferative activity of the compounds of the present disclosure on CAL51 cells was shown in Table 2.

TABLE 2
Anti-proliferative activity of CAL51 cells

| Compound Number | IC₅₀ (nM) |
|---|---|
| Compound 3 | 39 |

### Test Example 3: Degradation Activity Experiment on Intracellular GSPT1 Protein

### 1. Experimental Instruments and Materials

### Instruments and Equipment

| Instrument Name | Equipment manufacturer | Model |
|---|---|---|
| Centrifuge | Eppendorf | 5800 |
| Cytometer | Count star | IC1000 |
| 4° C. refrigerator | Thermo | YG-330L |
| Inverted microscope | Nikon | TS2 |
| Cell incubator | Thermo | 1IVIOS 250i |
| Compound dilution and | PerkinElmer | Echo |
| loading apparatus | | |
| Azure WB Imaging System | Azure biosystems | Sapphire |

### Experimental Reagents and Consumables

| Name | Item number | Brand |
|---|---|---|
| DMEM medium | 11995-065 | Gibco |
| FBS | 10099-141C | Gibco |
| Trypsin-EDTA (0.25%), phenol red | 25200056 | Gibco |
| PBS | SH30256.01 | Cytiva |
| Anti-α-Tubulin antibody produced in | T6074-200UL | SIGMA |
| mouse | | |
| Anti-GSPT1 antibody produced in rabbit | HPA052488-100UL | SIGMA |
| Goata-mouse IRDye 680RD | P/N 925-68070 | Li-COR |
| Goata-rabbit IRDye 800CW | P/N 925-32211 | Li-COR |
| 4% paraformaldehyde/universal tissue | BL539A | White |
| fixative | | shark |
| ® (PBS) Blocking Buffer | 927-70001-500 mL | Li-COR |
| 384-well plate | 354663 | Corning |
| Triton X-100 | SLCD3084 | SIGMA |

2 [symbol] Experimental Procedure

### (1) Cell Plating

The medium of CAL51 (CBP60360, Kebai) was removed, and PBS was added for rinsing once, and then digested with trypsin (Trypsin-EDTA (0.25%)) for 5 minutes. After digestion, 10 mL of complete medium (DMEM containing 10% FBS+10% FBS) was added to neutralize trypsin, and the cells were then pipetted, collected, centrifuged at 300 g for 5 min. The cell density was adjusted to 266,667 cells/mL. 30 μL of the cell suspension was added to a 384 well plate. The plate was centrifuged at 300 g for 1 min and then incubated in a cell incubator overnight.

### (2) Add Drug to the Cells

Compounds of the present disclosure were dissolved in DMSO, with a mother liquor storage concentration of 10 mM. The gradient dilution of compound mother liquor was carried out through the dose-response program of the compound dilution and loading instrument. The total experimental volume of the dilution program was 30 μL, with the initial concentration of 100 μM for the tested compound. A4-fold dilution was performed, resulting in 10 concentration points. The positive control compound was CC-885 in Test Example 2, the negative control was DMSO, and the DMSO content of all wells was 0.3%. After the procedure, then the plate was centrifuged and incubated in a carbon dioxide incubator for 6 hours.

### (3) Incubate Antibodies

After 6 hours of administration, the supernatant was discarded, the cells were washed with 50 μL of cold PBS, and then the cells were fixed with 50 μL/well of 4% paraformaldehyde/universal tissue fixation solution, and incubated at room temperature for 1 hour. The paraformaldehyde was discarded and 50 μL/well PBS-0.1% Triton X-100 was added to permeate cells for 30 minutes. PBS-Triton was discarded, 50 μL/well of blocking buffer (927-70001, Li-COR) was added, and incubated at room temperature for 1 hour. The blocking buffer was removed, and 30 μL/well of rabbit anti-GSPT1 (1: 300) (HPA052488, Sigma), mouse anti-α-Tubulin (1: 2000) (T6074, Sigma) primary antibody mixed antibody was added, and incubated at 4° C. overnight. The well was washed with 50 μL/well PBST (PBS containing 0.1% Tween-20) and soaked for 10 minutes per round for 4 times. 30 μL/well of goat anti-mouse 680RD (P/N 925-68070, Li-COR) and goat anti-rabbit 800CW (P/N 925-32211, Li-COR) secondary antibody mixture (1: 5000) was added and incubated at room temperature away from light for 1-1.5 hours. The well was washed with 50 μL/well PBST and soaked for 10 min per round for 4 times. The centrifuge plate was turned upside down, covered with toilet paper, then centrifuged at 1000 rpm/min for 1 minute, and the bottom of the plate was dried. A 384-well plate was scanned with an Azure WB imaging system (Sapphire) and the data was read.

### 3. Data Analysis

The GSPT1 protein degradation activity of the compounds of the present disclosure in CAL51 cells was measured by the above assay, and the protein degradation rate of each sample well was calculated based on the raw data.

$$Degradation rate ( \% ) = 100 \% \times ( 1-sample \text{reading/DMSO} reference mean reading )$$

Sample reading: refers to the signal value of the experimental group;
DSMO reference mean reading; refers to the mean signal value of the DSMO control group. No test compound was added to the positive control group, and other operations were consistent with those of the experimental group.

Data analysis was performed by GraphPad Prism 9. The concentration-effect curves were obtained using a nonlinear four-parameter fitting, and DC₅₀ of compounds was calculated:

$$Y = Bottom + ( Top - Bottom \text{)/(} 1 + 10 \hat{} ( ( Log {DC}_{50} - X ) * HillSlope ) )$$

Wherein:
X: Log of the compound concentration.
Y: degradation rate (%);
Bottom is the minimum percent degradation:
Top is the maximum percent degradation:
HillSlope is the slope coefficient of the curve.

The DC₅₀ values measured for the compounds of the present disclosure were shown in Table 3.

TABLE 3
GSPT1 protein degradation activity

| Compound Number | Maximum degradation rate (%) | DC50 (nM) |
|---|---|---|
| Compound 2 | 95.4 | 86.3 |
| Compound 3 | 98.3 | 25.4 |
| Compound 4 | 85.3 | 144 |
| Compound 5 | 96.7 | 20 |
| Compound 8 | 94.6 | 13 |
| Compound 9 | 69.6 | 1460 |
| Compound 13 | 96.3 | 135 |

1. A compound of formula (I) or a pharmaceutically acceptable salt thereof, 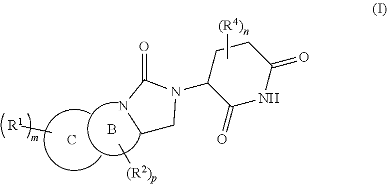
wherein,
ring B is selected from 5- to 6-membered heteroaromatic ring and 5- to 8-membered heterocyclic ring;
ring C is selected from 5- to 6-membered heteroaromatic ring, 5- to 8-membered heterocyclic ring, benzene ring, and C₅-C₈ saturated or partially saturated carbocyclic ring;
R¹ and R² are each independently selected from the following group;
(a) halogen, ═O, CN, NO₂, —OR^{b}, —N(R^{b})₂, —S(O)R^{b}, —SO₂R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 8-membered heterocyclyl, C₆-C₁₀ aryl or 5- to 10-membered heteroaryl is optionally substituted with R^{a};
or,
(b) 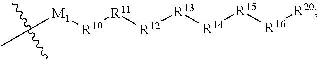
M₁ is selected from bond, —NR^{b}—, —C(O)—, —C(O)O—, —SO₂—, —S(O)—, —O—, —S—, —C(O)NR^{b}—, —C(═NR^{b})—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5-10 membered heteroarylene is optionally substituted with R^{a};
R¹⁰, R¹¹, R¹², R¹³, R¹⁴, R¹⁵, and R¹⁶ are independently selected from bond, —NR^{b}—, —C(O)—, —C(O)O—, —SO₂—, —S(O)—, —O—, —S—, —NR^{b}C(O)—, —C(═NR^{b})—, —C(S)—, —P(O)(OR)O—, —P(O)(OR^{b})—, 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, C₃-C₁₀ cycloalkylene, 4- to 9-membered heterocyclylene, C₆-C₁₀ arylene, or 5- to 10-membered heteroarylene is optionally substituted with R^{a};
R²⁰ is selected from H, halogen, CN, —OR^{b}, —N(R^{b})₂, —S(O)R^{b}, —SO₂R^{b}, —C(O)R^{b}, —C(O)OR^{b}, —OC(O)R^{b}, —C(O)N(R^{b})₂, —NR^{b}C(O)R^{b}, 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, and 5- to 10-membered heteroaryl, wherein the 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, or 5- to 10-membered heteroaryl is optionally substituted with R^{a};
each R⁴ is selected from halogen, CN, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{a};
each R^{a} is independently selected from halogen, CN, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{c};
each R^{b} is independently selected from H, halogen, CN, OH, NH₂, 2- to 10-membered heteroalkyl, C₁-C₁₀ alkyl, C₃-C₁₀ cycloalkyl, 4- to 8-membered heterocyclyl, C₆-C₁₀ aryl, and 5- to 10-membered heteroaryl, wherein the OH, NH₂, 2- to 10-membered heteroalkyl, C₁-C₁₀ alkyl, C₃-C₁₀ cycloalkyl, 4- to 8-membered heterocyclyl, C₆-C₁₀ aryl, or 5- to 10-membered heteroaryl is optionally substituted with R^{c};
each R^{c} is independently selected from halogen, CN, OH, NH₂, 2- to 10-membered heteroalkyl, C₁-C₁₀ alkyl, C₃-C₁₀ cycloalkyl, and 4- to 8-membered heterocyclyl, wherein the OH, NH₂, 2- to 10-membered heteroalkyl, C₁-C₁₀ alkyl, C₃-C₁₀ cycloalkyl, or 4- to 8-membered heterocyclyl is optionally substituted with R^{d};
each R^{d} is independently selected from halogen, CN, OH, NH₂, and C₁-C₆ alkyl;
n is independently selected from 0, 1, 2, 3, and 4;
m and p are independently selected from 0, 1, 2, 3, 4, 5, and 6.

2. The compound of formula (I) or the pharmaceutically acceptable salt thereof according to claim 1, wherein ring B is selected from 5- to 6-membered heteroaromatic ring and 5- to 6-membered heterocyclic ring; or
ring B is selected from 5- to 6-membered heteroaromatic ring; or
wherein ring C is selected from 5- to 6-membered heteroaromatic ring, 5- to 6-membered heterocyclic ring, phenyl ring, and C₅-C₆ saturated or partially saturated carbocyclic ring; or
ring C is selected from 5- to 6-membered heteroaromatic ring and benzene ring.

3. (canceled)

4. The compound of formula (I) or the pharmaceutically acceptable salt thereof according to claim 1, wherein R¹ and R² are independently selected from halogen, CN, NO₂, —OR^{b}, —N(R^{b})₂, —S(O)R^{b}, —SO₂R^{b}, C₁-C₁₀ alkyl, and C₃-C₁₀ cycloalkyl, wherein the C₁-C₁₀ alkyl or C₃-C₁₀ cycloalkyl is optionally substituted with R^{a}.

5. The compound of formula (I) or the pharmaceutically acceptable salt thereof according to claim 1, wherein R¹ and R² are independently selected from 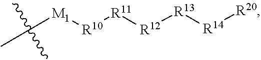
wherein M₁, R¹⁰, R¹¹, R¹², R¹³, R¹⁴, R²⁰ are as defined in claim 1; or
wherein R¹ and R² are independently selected from 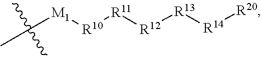
wherein R¹¹, R¹² and R¹³ are independently selected from bond, —C(O)—, —C(O)O—, —O—, —S—, —C(O)NR^{b}—, and —NR^{b}—, M₁, R¹⁰, R¹⁴, R²⁰, and R^{b} are as defined in claim 1; or
wherein R¹ and R² are independently selected from 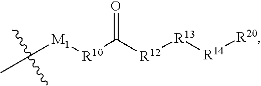
wherein M₁, R¹⁰, R¹², R¹³, R¹⁴, and R²⁰ are as defined in claim 1.

6. (canceled)

7. (canceled)

8. The compound of formula (I) or the pharmaceutically acceptable salt thereof according to claim 1, wherein M₁ is selected from bond, —NH—, —CH₂—, —CH₂CH₂—, —C(O)—, —C(O)O—, —O—, —S—, and —C(O)NH—.

9. The compound of formula (I) or the pharmaceutically acceptable salt thereof according to claim 1, wherein R¹ and R² are independently selected from 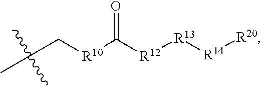
wherein R¹⁰ and R¹² are independently selected from bond, —C(O)O—, —O—, —S—, —C(O)NR^{b}—, —NR^{b}—, C₁-C₃ alkylene, and C₂-C₃ alkynylene, wherein the C₁-C₃ alkylene or C₂-C₃ alkynylene is optionally substituted with R^{a}; R¹³, R¹⁴, R²⁰, R^{a}, and R^{b} are as defined in claim 1.

10. The compound of formula (I) or the pharmaceutically acceptable salt thereof according to claim 1, wherein R¹³ is selected from C₃-C₁₀ cycloalkylene, 4- to 9-membered heterocyclylene, C₆-C₁₀ arylene, and 5- to 10-membered heteroarylene, wherein the C₃-C₁₀ cycloalkylene, 4- to 9-membered heterocyclylene, C₆-C₁₀ arylene, or 5- to 10-membered heteroarylene is optionally substituted with R^{a}; or
R¹³ is selected from C₃-C₆ cycloalkylene, 5- to 9-membered heterocyclylene, phenyl, and 5- to 6-membered heteroarylene, wherein the C₃-C₆ cycloalkylene, 5- to 9-membered heterocyclylene, phenyl, or 5- to 6-membered heteroarylene is optionally substituted with R^{a}.

11. The compound of formula (I) or the pharmaceutically acceptable salt thereof according to claim 1, wherein R¹⁴ is selected from bond, —O—, —NR^{b}—, —C(O)NR^{b}—, 2- to 6-membered heteroalkylene, C₁-C₆ alkylene, C₂-C₆ alkenylene, C₂-C₆ alkynylene, 4- to 6-membered heterocyclylene, C₆-C₁₀ arylene, and 5- to 6-membered heteroarylene, wherein the 2- to 6-membered heteroalkylene, C₁-C₆ alkylene, C₂-C₆ alkenylene, C₂-C₆ alkynylene, 4- to 6-membered heterocyclylene, C₆-C₁₀ arylene, or 5- to 6-membered heteroarylene is optionally substituted with R^{a}; or
R¹⁴ is selected from bond, —O—, —NR^{b}—, —C(O)NR^{b}—, 2- to 6-membered heteroalkylene, C₁-C₆ alkylene, C₂-C₆ alkenylene, and C₂-C₆ alkynylene, wherein the C₁-C₆ alkylene, C₂-C₆ alkenylene, or C₂-C₆ alkynylene is optionally substituted with R^{a}.

12. The compound of formula (I) or the pharmaceutically acceptable salt thereof according to claim 1, wherein R¹⁰, R¹¹, R¹², R¹³, R¹⁴, R¹⁵, and R¹⁶ are independently selected from bond, —C(O)—, —C(O)O—, —O—, —S—, —C(O)NR^{b}—, —NR^{b}—, 2- to 10-membered heteroalkylene, C₁-C₁₀ alkylene, C₂-C₁₀ alkenylene, C₂-C₁₀ alkynylene, 4- to 9-membered heterocyclylene, C₆-C₁₀ arylene, and 5- to 10-membered heteroarylene, wherein the 2- to 10-membered heteroalkylene, C₁-C₁₀ alkylene, C₂-C₁₀ alkenylene, C₂-C₁₀ alkynylene, 4- to 9-membered heterocyclylene, C₆-C₁₀ arylene, or 5- to 10-membered heteroarylene is optionally substituted with R^{a}.

13. The compound of formula (I) or the pharmaceutically acceptable salt thereof according to claim 1, wherein R²⁰ is selected from H, 2- to 10-membered heteroalkyl, C₁-C₁₀ alkyl, C₂-C₁₀ alkenyl, C₂-C₁₀ alkynyl, C₃-C₁₀ cycloalkyl, 4- to 9-membered heterocyclyl, C₆-C₁₀ aryl, and 5- to 10-membered heteroaryl, wherein the 2- to 10-membered heteroaryl, C₁-C₁₀ alkyl, C₂-C₁₀ alkenyl, C₂-C₁₀ alkynyl, C₃-C₁₀ cycloalkyl, 4- to 9-membered heteroalkyl, C₆-C₁₀ aryl, or 5- to 10-membered heteroaryl is optionally substituted with R^{a}; or
R²⁰ is selected from H, —N(R^{b})₂, C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, and 5- to 10-membered heteroaryl, wherein the C₁-C₆ alkyl, C₂-C₆ alkenyl, C₂-C₆ alkynyl, C₃-C₁₀ cycloalkyl, 4- to 9-membered heterocyclyl, C₆-C₁₀ aryl, or 5- to 10-membered heteroaryl is optionally substituted with R^{a}.

14. The compound of formula (I) or the pharmaceutically acceptable salt thereof according to claim 1, wherein, R⁴ is independently selected from halogen, CN, OH, NH₂, 2- to 10-membered heteroalkyl, and C₁-C₁₀ alkyl, wherein the OH, NH₂, 2- to 10-membered heteroalkyl, or C₁-C₁₀ alkyl is optionally substituted with R^{a}.

15. The compound of formula (I) or the pharmaceutically acceptable salt thereof according to claim 1, wherein, each R^{a} is independently selected from halogen, CN, OH, NH₂, C₁-C₁₀ alkyl, C₃-C₁₀ cycloalkyl, and 4- to 8-membered heterocyclyl, wherein the OH, NH₂, C₁-C₁₀ alkyl, C₃-C₁₀ cycloalkyl, or 4- to 8-membered heterocyclyl is optionally substituted with R^{c}; or
each R^{a} is independently selected from halogen, CN, OH, NH₂, and C₁-C₁₀ alkyl, wherein the OH, NH₂, or C₁-C₁₀ alkyl is optionally substituted with R^{c}; or
wherein each R^{c} is independently selected from halogen, CN, OH, NH₂, and C₁-C₆ alkyl, wherein the C₁-C₆ alkyl is optionally substituted with R^{d}.

16. (canceled)

17. The compound of formula (I) or the pharmaceutically acceptable salt thereof according to claim 1, wherein m and p are independently selected from 0, 1, and 2; or
m is 1, and p is 0; or
m is 0, and p is 1; or
wherein n is selected from 0 and 1: or
n is 0.

18. (canceled)

19. The compound of formula (I) or the pharmaceutically acceptable salt thereof according to claim 1, wherein the compound of formula (I) or the pharmaceutically acceptable salt thereof is selected from a compound of formula (II) or a pharmaceutically acceptable salt thereof, 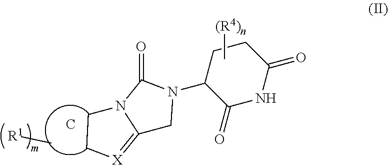
wherein, X is selected from N and CH, wherein the CH is optionally substituted with R²; R¹, R², R⁴, m, and n are as defined in claim 1.

20. The compound of formula (I) or the pharmaceutically acceptable salt according to claim 1, wherein the compound of formula (II) or the pharmaceutically acceptable salt thereof is selected from a compound of formula (II-1) or (II-2) or a pharmaceutically acceptable salt thereof, 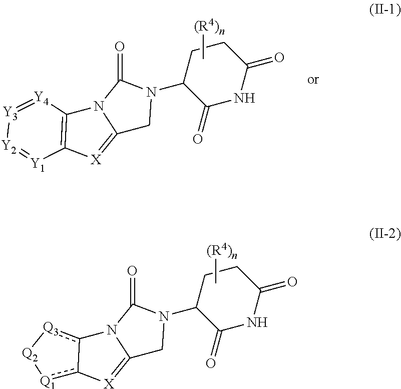
wherein “ [symbol] ” represents a single bond or a double bond; X is selected from N and CH, wherein the CH is optionally substituted with R²; Y₁, Y₂, Y₃, and Y₄ are independently selected from N and CH, wherein the CH is optionally substituted with R¹; Q₁, Q₂, and Q₃ are independently selected from O, S, NH, CH₂, N, and CH, wherein the NH, CH₂, or CH is optionally substituted with R¹; R¹, R², R⁴, and n are as defined in claim 1.

21. The compound of formula (I) or the pharmaceutically acceptable salt thereof according to claim 1, wherein the compound of formula (I) or the pharmaceutically acceptable salt thereof is selected from the following compounds or pharmaceutically acceptable salts thereof,

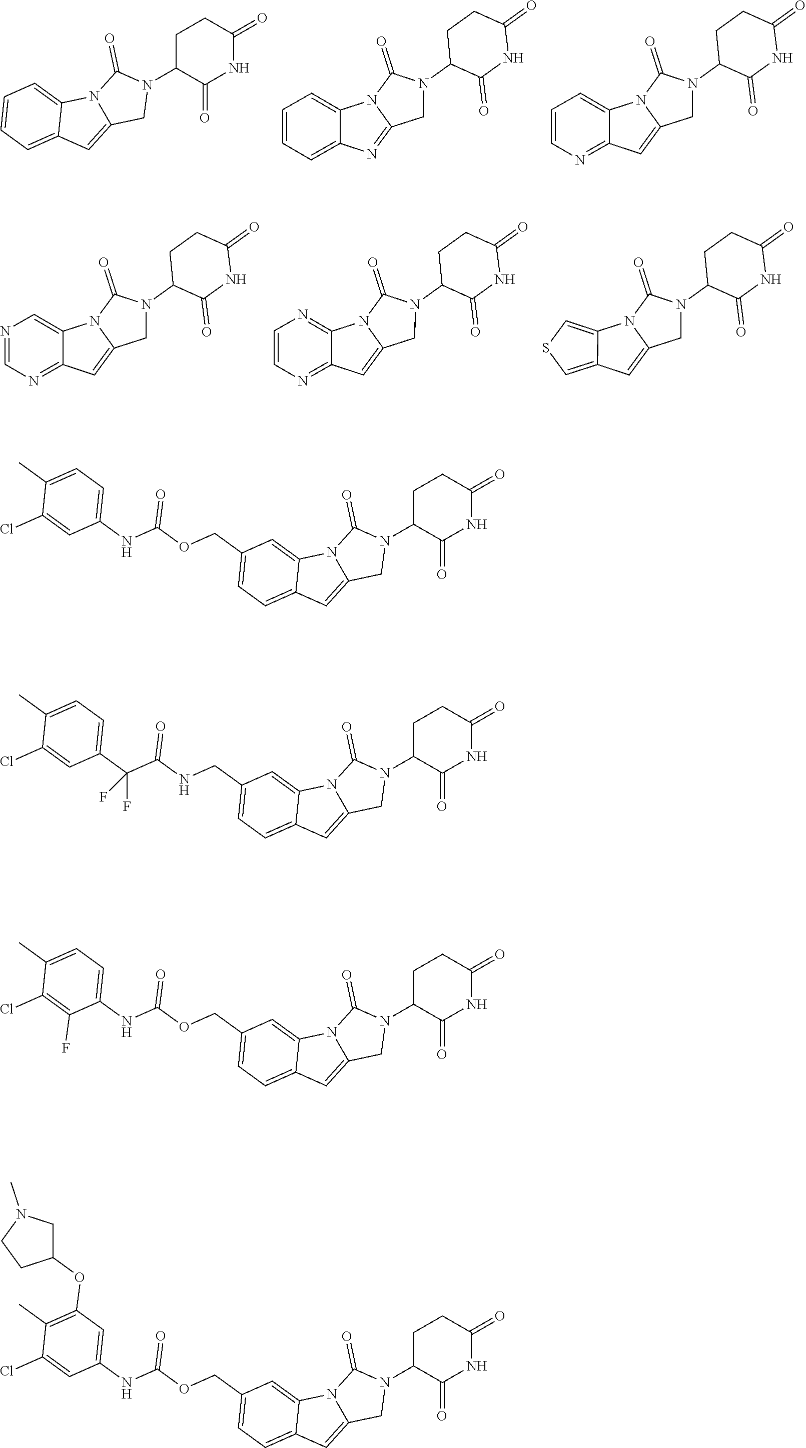

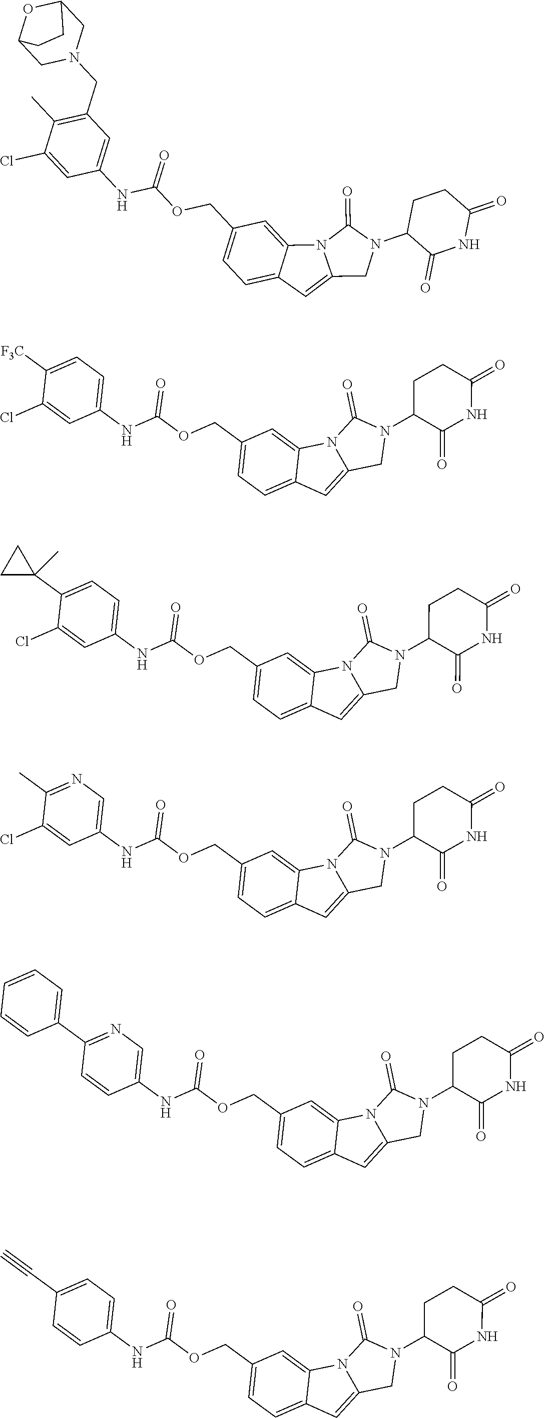 [SMILES: Cc1c(Cl)cc(NC(=O)OCc2ccc3cc4n(c3c2)C(=O)N(C2CCC(=O)NC2=O)C4)cc1CN1CC2CCC(C1)O2 ; O=C1CCC(N2Cc3cc4ccc(COC(=O)Nc5ccc(C(F)(F)F)c(Cl)c5)cc4n3C2=O)C(=O)N1 ; CC1(c2ccc(NC(=O)OCc3ccc4cc5n(c4c3)C(=O)N(C3CCC(=O)NC3=O)C5)cc2Cl)CC1 ; Cc1ncc(NC(=O)OCc2ccc3cc4n(c3c2)C(=O)N(C2CCC(=O)NC2=O)C4)cc1Cl ; O=C1CCC(N2Cc3cc4ccc(COC(=O)Nc5ccc(-c6ccccc6)nc5)cc4n3C2=O)C(=O)N1 ; C#Cc1ccc(NC(=O)OCc2ccc3cc4n(c3c2)C(=O)N(C2CCC(=O)NC2=O)C4)cc1]

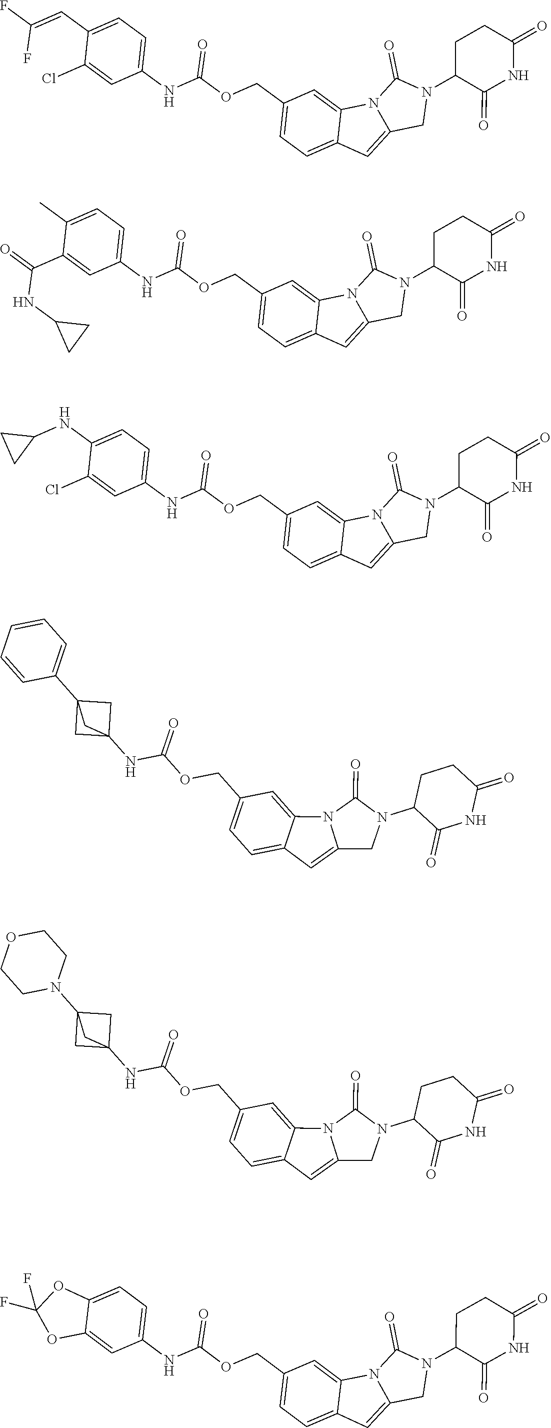 [SMILES: O=C1CCC(N2Cc3cc4ccc(COC(=O)Nc5ccc(C=C(F)F)c(Cl)c5)cc4n3C2=O)C(=O)N1 ; Cc1ccc(NC(=O)OCc2ccc3cc4n(c3c2)C(=O)N(C2CCC(=O)NC2=O)C4)cc1C(=O)NC1CC1 ; O=C1CCC(N2Cc3cc4ccc(COC(=O)Nc5ccc(NC6CC6)c(Cl)c5)cc4n3C2=O)C(=O)N1 ; O=C1CCC(N2Cc3cc4ccc(COC(=O)NC56CC(c7ccccc7)(C5)C6)cc4n3C2=O)C(=O)N1 ; O=C1CCC(N2Cc3cc4ccc(COC(=O)NC56CC(N7CCOCC7)(C5)C6)cc4n3C2=O)C(=O)N1 ; O=C1CCC(N2Cc3cc4ccc(COC(=O)Nc5ccc6c(c5)OC(F)(F)O6)cc4n3C2=O)C(=O)N1]

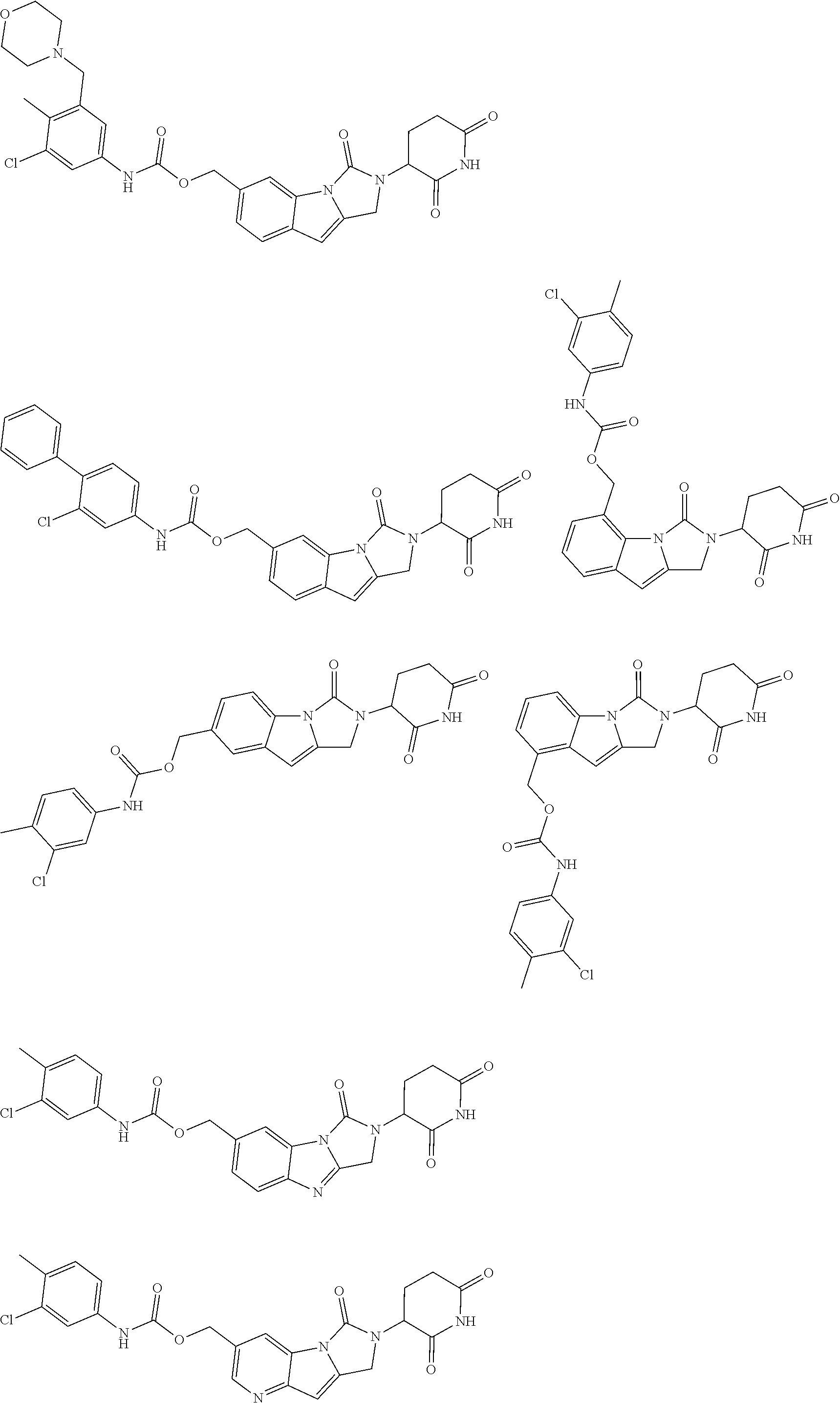

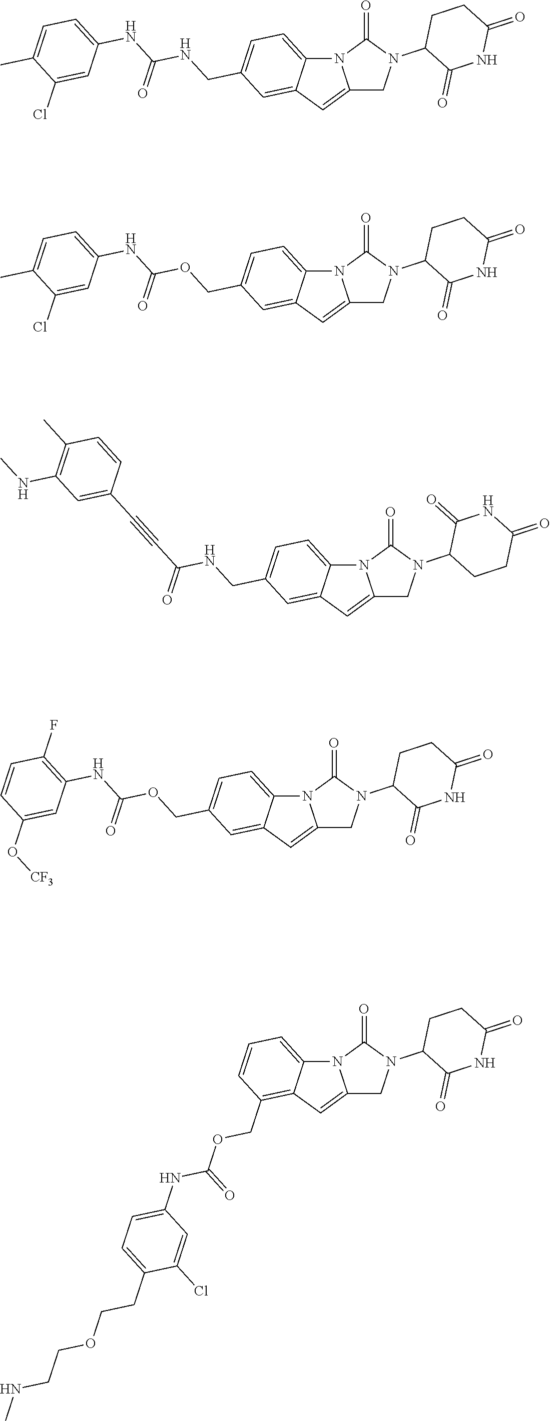 [SMILES: Cc1ccc(NC(=O)NCc2ccc3c(c2)cc2n3C(=O)N(C3CCC(=O)NC3=O)C2)cc1Cl ; Cc1ccc(NC(=O)OCc2ccc3c(c2)cc2n3C(=O)N(C3CCC(=O)NC3=O)C2)cc1Cl ; CNc1cc(C#CC(=O)NCc2ccc3c(c2)cc2n3C(=O)N(C3CCC(=O)NC3=O)C2)ccc1C ; O=C1CCC(N2Cc3cc4cc(COC(=O)Nc5cc(OC(F)(F)F)ccc5F)ccc4n3C2=O)C(=O)N1 ; CNCCOCCc1ccc(NC(=O)OCc2cccc3c2cc2n3C(=O)N(C3CCC(=O)NC3=O)C2)cc1Cl]

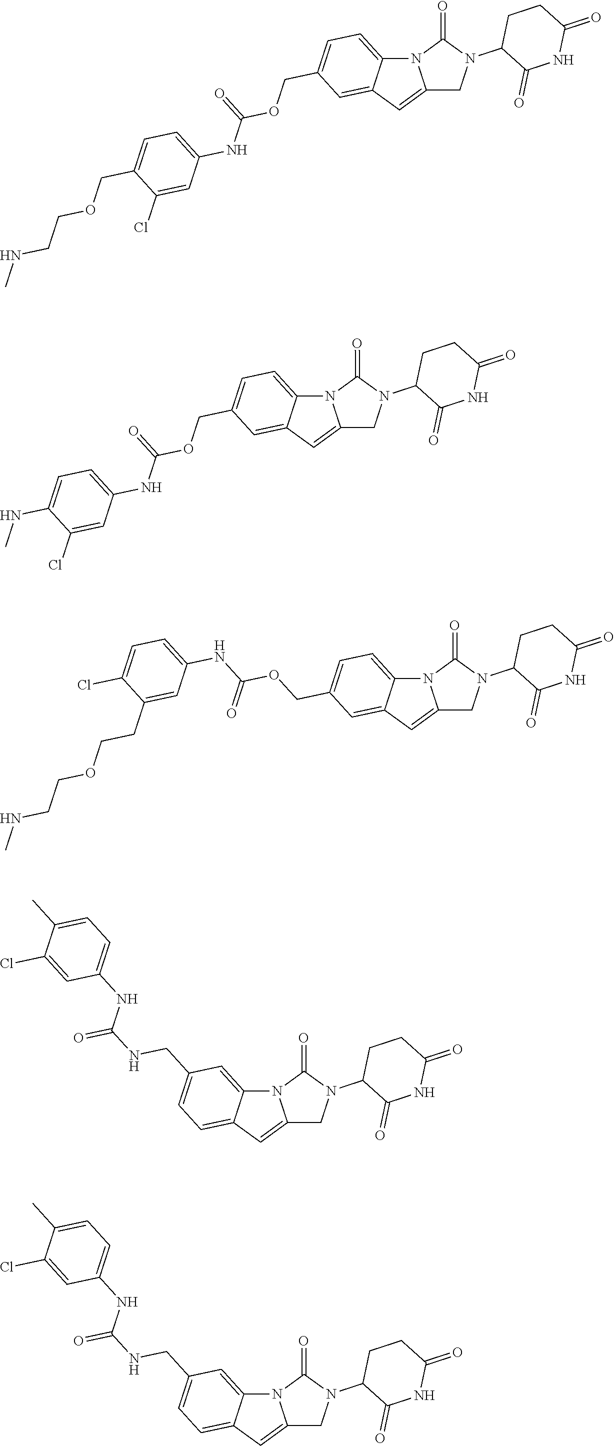 [SMILES: CNCCOCc1ccc(NC(=O)OCc2ccc3c(c2)cc2n3C(=O)N(C3CCC(=O)NC3=O)C2)cc1Cl ; CNc1ccc(NC(=O)OCc2ccc3c(c2)cc2n3C(=O)N(C3CCC(=O)NC3=O)C2)cc1Cl ; CNCCOCCc1cc(NC(=O)OCc2ccc3c(c2)cc2n3C(=O)N(C3CCC(=O)NC3=O)C2)ccc1Cl ; Cc1ccc(NC(=O)NCc2ccc3cc4n(c3c2)C(=O)N(C2CCC(=O)NC2=O)C4)cc1Cl ; Cc1ccc(NC(=O)NCc2ccc3cc4n(c3c2)C(=O)N(C2CCC(=O)NC2=O)C4)cc1Cl]

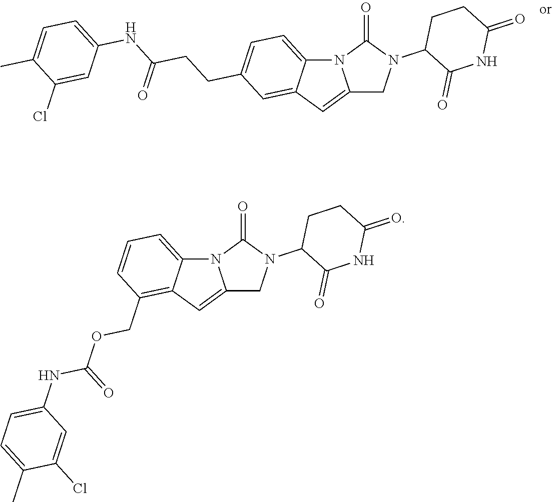 [SMILES: Cc1ccc(NC(=O)CCc2ccc3c(c2)cc2n3C(=O)N(C3CCC(=O)NC3=O)C2)cc1Cl ; Cc1ccc(NC(=O)OCc2cccc3c2cc2n3C(=O)N(C3CCC(=O)NC3=O)C2)cc1Cl]

22. A pharmaceutical composition, comprising the compound of formula (I) or the pharmaceutically acceptable salt thereof according to claim 1, and a pharmaceutically acceptable excipient.

23. (canceled)

24. A method for treating abnormal cell proliferation diseases in a mammal, comprising administering to a mammal in need thereof a therapeutically effective amount of the compound of formula (I) or the pharmaceutically acceptable salt thereof according to claim 1.

25. The method according to claim 24, wherein the abnormal cell proliferation disease is cancer.

26. The method according to claim 25, wherein the cancer is selected from solid tumors, adenocarcinoma and hematoma.
